SEQUENCE LISTING (18 sequences)

SEQ ID NO: 1 - DNA, 20 bp, Artificial sequence
  Single strand DNA oligonucleotide
gagtgtgtttgtcaccgttg

SEQ ID NO: 2 - DNA, 20 bp, Artificial sequence
  Single strand DNA oligonucleotide
tactcgttggtcgagagctg

SEQ ID NO: 3 - DNA, 20 bp, Artificial sequence
  Single strand DNA oligonucleotide
gctcacccagtgacaactca

SEQ ID NO: 4 - DNA, 20 bp, Artificial sequence
  Single strand DNA oligonucleotide
cccagcagcttcaggtactc

SEQ ID NO: 5 - DNA, 20 bp, Artificial sequence
  Single strand DNA oligonucleotide
tggtaaaccttcgggctatg

SEQ ID NO: 6 - DNA, 20 bp, Artificial sequence
  Single strand DNA oligonucleotide
ccattcctttcagtggcaat

SEQ ID NO: 7 - DNA, 20 bp, Artificial sequence
  Single strand DNA oligonucleotide
cctcaggtgacctctccaag

SEQ ID NO: 8 - DNA, 20 bp, Artificial sequence
  Single strand DNA oligonucleotide
tactatgccatcgcttgctg

SEQ ID NO: 9 - DNA, 20 bp, Artificial sequence
  Single strand DNA oligonucleotide
ggcagctgaagaagcaaatc

SEQ ID NO: 10 - DNA, 20 bp, Artificial sequence
  Single strand DNA oligonucleotide
tcaaccgatactgcaagctg

SEQ ID NO: 11 - DNA, 20 bp, Artificial sequence
  Single strand DNA oligonucleotide
catcacattcctgaccatcg

SEQ ID NO: 12 - DNA, 20 bp, Artificial sequence
  Single strand DNA oligonucleotide
acgtgcttctctgccttgtt

SEQ ID NO: 13 - DNA, 20 bp, Artificial sequence
  Single strand DNA oligonucleotide
agcctgtggaaatggcatag

SEQ ID NO: 14 - DNA, 20 bp, Artificial sequence
  Single strand DNA oligonucleotide
ctctcatgaaggaggccaag

SEQ ID NO: 15 - DNA, 20 bp, Artificial sequence
  Single strand DNA oligonucleotide
ggaaaccatgccagatgacc

SEQ ID NO: 16 - DNA, 20 bp, Artificial sequence
  Single strand DNA oligonucleotide
actttctgggagatggccaa

SEQ ID NO: 17 - DNA, 20 bp, Artificial sequence
  Single strand DNA oligonucleotide
ggtgcttatcaggtgccaag

SEQ ID NO: 18 - DNA, 20 bp, Artificial sequence
  Single strand DNA oligonucleotide
gttgtcagctcaaggatgcc

(19) United States
(12) Patent Application Publication
(10) Pub. No.: US 2022/0040144 A1
(43) Pub. Date: Feb. 10, 2022

(54) COMPOSITIONS AND METHODS FOR THE TREATMENT OF CUTANEOUS T CELL LYMPHOMA (CTCL)

(71) Applicants: The State of Israel, Ministry of Agriculture & Rural Development, Agricultural Research Organization, Rishon-LeZion (IL); Mor Research Applications Ltd., Ramat-Gan (IL)

(72) Inventors: Hinanit KOLTAI, Rishon-LeZion (IL); Moran MAZUZ, Rishon-LeZion (IL); Dvora NAMDAR, Beit Gamliel (IL); Iris AMITAY-LAISH, Kiryat Ono (IL); Emmilia HODAK, Tel Aviv (IL); Lilach MOYAL ELCHARAR, Herzliya (IL)

(73) Assignees: The State of Israel, Ministry of Agriculture & Rural Development, Agricultural Research Organization, Rishon-LeZion (IL); Mor Research Applications Ltd., Rishon-LeZion (IL)

(21) Appl. No.: 17/413,584

(22) PCT Filed: Dec. 12, 2019
(86) PCT No.: PCT/IL2019/051365

§ 371 Date: Jun. 14, 2021

Related U.S. Application Data
(60) Provisional application No. 62/791,951, filed on Jan. 14, 2019.
(60) Provisional application No. 62/778,319, filed on Dec. 12, 2018.

(51) Int. Cl. A61K 31/352; A61K 31/05; A61P 35/00

(52) U.S. Cl. CPC A61K 31/352; A61P 35/00; A61K 31/05

(57) ABSTRACT

Compositions and methods for the treatment of cutaneous T cell lymphoma (CTCL) are provided. The compositions and uses thereof comprise cannabinoids.

### RELATED APPLICATIONS

This application claims priority from U.S. Provisional Patent Application No. 62/778,319 filed on 12 Dec. 2018 and from U.S. Provisional Patent Application No. 62/791,951 filed on 14 Jan. 2019, the contents of which are incorporated herein by reference in their entirety.

### SEQUENCE LISTING STATEMENT

The ASCII file, entitled 80324 Sequence Listing.txt, created on 12 Dec. 2019, comprising 8,192 bytes, submitted concurrently with the filing of this application is incorporated herein by reference.

### FIELD AND BACKGROUND OF THE INVENTION

The present invention, in some embodiments thereof, relates to compositions and methods for the treatment of cutaneous T cell lymphoma.

Cutaneous T-cell lymphomas (CTCL) are the most frequent primary lymphomas of the skin. The CTCL group comprises mycosis fungoides (MF), transformed mycosis fungoides, Sézary Syndrome, Lymphomatoide Papulosis, CD30+ cutaneous lymphomas, with mycosis fungoides (MF) being the most prevalent clinical form accounting for around 60% of new cases. In early disease stages, which can last several years, MF presents as flat erythematous skin patches resembling inflammatory diseases such as dermatitis or psoriasis. In later stages, MF lesions gradually form plaques and overt tumors and may disseminate to lymph nodes and internal organs. The early skin lesions of this disease contain numerous inflammatory cells, including a large quantity of T cells with a normal phenotype as well as a small population of T cells with a malignant phenotype. Sézary Syndrome is an aggressive form of CTCL characterized by a clonal expansion of CD4^{±}/CD45RO⁺ T cells and the appearance of these malignant T cells in the blood. The biology and etiology of CTCL remains poorly understood. Today, CTCL therapy includes skin creams, light therapy, chemotherapy, biological therapy, radiation therapy and stem cell transplantation.

Marijuana (Cannabis sativa) contains more than 500 constituents, among them more than a hundred terpenophenolic compounds termed phytocannabinoids [ElSohly et al., Phytochemistry of Cannabis sativa L. Phytocannabinoids, Springer (2017) 1-36]. An increasing number of studies have shown that phytocannabinoids can prevent proliferation, metastasis, angiogenesis and induce apoptosis in a variety of cancer cell types including breast, lung, prostate, skin, intestine, glioma, and others (e.g. International Patent Application Publication No: WO2016097831, EP Patent No: EP1071417, U.S. Pat. Nos. 8,632,825 and 8,632,825).

Additional background art includes:

International Patent Application Publication Nos. WO2009/004302; WO2018/163164; WO2018/163163; WO2017/158609; WO2017/161387; WO2011/066371 and WO/2008/109027;

US Patent Application Publication No: US20150150952; and

U.S. Pat. Nos. 9,526,752, 9,084,771 and 6,410,588.

### SUMMARY OF THE INVENTION

According to an aspect of some embodiments of the present invention there is provided a composition selected from the group consisting of:

(i) a composition comprising 40-60% tetrahydrocannabinol (THC) and/or 35-55% tetrahydrocannabinolic acid (THCA);

(ii) a composition comprising at least two of cannabinoids selected from the group consisting of cannabidiol (CBD), cannabichromene (CBC) and cannabigerol (CBG), wherein a concentration of the CBD, the CBC and/or the CBG in the composition is 5-30% CBD, 0.1-10% CBC and/or 0.1-15% CBG;

(iii) a composition comprising at least one cannabinoid selected from the group consisting of cannabidiol (CBD), cannabichromene (CBC), tetrahydrocannabinol (THC) and cannabigerol (CBG), wherein the composition comprises at least 50% CBG and/or wherein the composition is devoid of tetrahydrocannabinolic acid (THCA); and

(iv) a composition comprising at least 90% cannabidiol (CBD) and at least one cannabinoid other than the CBD,

for use in treating cutaneous T cell lymphoma (CTCL) in a subject in need thereof.

According to an aspect of some embodiments of the present invention there is provided a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least two cannabinoids selected from the group consisting of cannabidiol (CBD), cannabichromene (CBC) and cannabigerol (CBG), wherein a concentration of the CBD, the CBC and/or the CBG in the composition is 5-30% CBD, 0.1-10% CBC and/or 0.1-15% CBG; and

(iii) a composition comprising at least two cannabinoids selected from the group consisting of cannabidiol (CBD), cannabichromene (CBC), tetrahydrocannabinol (THC) and cannabigerol (CBG), wherein the composition comprises at least 50% CBG and/or wherein the composition is devoid of tetrahydrocannabinolic acid (THCA) and the inflammatory disease is not inflammatory bowel disease,

for use in treating an inflammatory disease in a subject in need thereof.

According to some embodiments of the invention, the composition for use further comprising a composition (iv) comprising at least 90% CBD and at least one cannabinoid other than the CBD.

According to an aspect of some embodiments of the present invention there is provided a composition comprising 50-90% cannabidiol (CBD), 10-40% cannabigerol (CBG) and 0.15-0.5% tetrahydrocannabinol (THC) for use in treating an inflammatory disease in a subject in need thereof.

According to an aspect of some embodiments of the present invention there is provided a composition comprising cannabidiol (CBD), cannabigerol (CBG) and tetrahydrocannabinol (THC) in molar ratios as listed in Table 5, for use in treating an inflammatory disease in a subject in need thereof.

According to an aspect of some embodiments of the present invention there is provided a composition selected from the group consisting of:

(i) a composition comprising 40-60% tetrahydrocannabinol (THC) and at least one cannabinoid selected form the group consisting of tetrahydrocannabinolic acid (THCA), cannabinol (CBN) and β-caryophyllene;

(ii) a composition comprising at least two cannabinoids selected from the group consisting of cannabidiol (CBD), cannabichromene (CBC) and cannabigerol (CBG), wherein a concentration of the CBD, the CBC and/or the CBG in the composition is 5-30% CBD, 0.1-10% CBC and/or 0.1-15% CBG; and

(iii) a composition comprising at least 50% cannabigerol (CBG) and at least one cannabinoid selected from the group consisting of cannabidiol (CBD), cannabichromene (CBC) and tetrahydrocannabinol (THC).

According to an aspect of some embodiments of the present invention there is provided an article of manufacture comprising the composition and a composition (iv) comprising at least 90% CBD and at least one cannabinoid other than the CBD.

According to an aspect of some embodiments of the present invention there is provided a composition comprising 50-90% cannabidiol (CBD), 10-40% cannabigerol (CBG) and 0.15-0.5% tetrahydrocannabinol (THC).

According to some embodiments of the invention, the composition further comprises 0.09-0.3% cannabichromene (CBC).

According to some embodiments of the invention, a total concentration of the CBD, the CBG the THC and/or the CBC in the composition is as listed in Table 5.

According to an aspect of some embodiments of the present invention there is provided a composition comprising cannabidiol (CBD), cannabigerol (CBG) and tetrahydrocannabinol (THC) in molar ratios as listed in Table 5.

According to some embodiments of the invention, the compositing further comprises cannabichromene (CBC) in a molar ratio as listed in Table 5.

According to an aspect of some embodiments of the present invention there is provided a composition comprising at least two cannabinoids selected from the group consisting of cannabidiol (CBD), cannabichromene (CBC), cannabigerol (CBG), tetrahydrocannabinol (THC), tetrahydrocannabinolic acid (THCA) and cannabinol (CBN), wherein a concentration of the CBD, the CBC, the CBG, the THC, the THCA and/or the CBN in the composition is 2.5-12% CBD, 0.5-2.2% CBC, 1-5% CBG, 14-43% THC, 13-40% THCA and/or 0.5-1.5% CBN.

According to some embodiments of the invention, a concentration of the CBD, the CBC, the CBG, the THC, the THCA and/or the CBN in the composition is as listed in Table 10.

According to an aspect of some embodiments of the present invention there is provided a method of treating a cutaneous T cell lymphoma (CTCL) in a subject in need thereof, the method comprising administering to the subject a therapeutically effective amount of a liquid chromatography fraction of a cannabis extract selected from the group consisting of:

(i) a liquid chromatography fraction comprising 40-60% tetrahydrocannabinol (THC) and/or 35-55% tetrahydrocannabinolic acid (THCA);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein the liquid chromatography fraction comprises at least % CBG and/or wherein the liquid chromatography fraction is devoid of tetrahydrocannabinolic acid (THCA); and

(iv) a liquid chromatography fraction comprising cannabidiol (CBD),

thereby treating the CTCL in the subject.

According to an aspect of some embodiments of the present invention there is provided a method of treating an inflammatory disease in a subject in need thereof, the method comprising administering to the subject a therapeutically effective amount of a liquid chromatography fraction of a cannabis extract selected from the group consisting of:

(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein the liquid chromatography fraction comprises at least 50% CBG and/or wherein the liquid chromatography fraction is devoid of tetrahydrocannabinolic acid (THCA), and wherein the liquid chromatography fraction comprises the CBD the liquid chromatography fraction comprises at least two of the cannabinoids; and (iv) a liquid chromatography fraction comprising cannabidiol (CBD), wherein the CBD is at least 90% CBD and/or wherein the liquid chromatography fraction is devoid of tetrahydrocannabinolic acid (THCA)

thereby treating the inflammatory disease in the subject.

According to an aspect of some embodiments of the present invention there is provided a method of treating cutaneous T cell lymphoma (CTCL) in a subject in need thereof, the method comprising administering to the subject a therapeutically effective amount of a composition selected from the group consisting of:

(i) a composition comprising 40-60% tetrahydrocannabinol (THC) and/or 35-55% tetrahydrocannabinolic acid (THCA);

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a composition comprising at least one cannabinoid selected from the group consisting of cannabidiol (CBD), cannabichromene (CBC), tetrahydrocannabinol (THC) and cannabigerol (CBG), wherein the composition comprises at least 50% CBG and/or wherein the composition is devoid of tetrahydrocannabinolic acid (THCA); and

(iv) a composition comprising cannabidiol (CBD),

thereby treating the CTCL in the subject.

According to an aspect of some embodiments of the present invention there is provided a method of treating an inflammatory disease in a subject in need thereof, the method comprising administering to the subject a therapeutically effective amount of a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; and

(iii) a composition comprising at least two cannabinoids selected from the group consisting of cannabidiol (CBD), cannabichromene (CBC), tetrahydrocannabinol (THC) and cannabigerol (CBG), wherein the composition comprises at least 50% CBG and/or wherein the composition is devoid of tetrahydrocannabinolic acid (THCA) and the inflammatory disease is not inflammatory bowel disease,

thereby treating the inflammatory disease in the subject.

According to some embodiments of the invention, the method further comprising administering to the subject a therapeutically effective amount of a composition (iv) comprising CBD.

According to an aspect of some embodiments of the present invention there is provided a composition comprising a liquid chromatography fraction of a cannabis extract selected from the group consisting of:
(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and/or cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and/or cannabigerol (CBG), wherein the liquid chromatography fraction comprises at least % CBG and/or wherein the liquid chromatography fraction is devoid of tetrahydrocannabinolic acid (THCA) and wherein the liquid chromatography fraction comprises the CBD the liquid chromatography fraction comprises at least two of the cannabinoids; and

(iv) a liquid chromatography fraction comprising cannabidiol (CBD), wherein the CBD is at least 90% CBD and/or wherein the liquid chromatography fraction is devoid of tetrahydrocannabinolic acid (THCA).

According to an aspect of some embodiments of the present invention there is provided a composition selected from the group consisting of:

(i) a composition comprising 40-60% tetrahydrocannabinol (THC) and at least one cannabinoid selected form the group consisting of tetrahydrocannabinolic acid (THCA), cannabinol (CBN) and β-caryophyllene;

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; and

(iii) a composition comprising at least 50% cannabigerol (CBG) and at least one cannabinoid selected from the group consisting of cannabidiol (CBD), cannabichromene (CBC) and tetrahydrocannabinol (THC).

According to an aspect of some embodiments of the present invention there is provided an article of manufacture comprising the composition and a composition (iv) comprising CBD.

According to some embodiments of the invention, the composition of (iii) is devoid of tetrahydrocannabinolic acid (THCA).

According to an aspect of some embodiments of the present invention there is provided a composition comprising at least one cannabinoid selected from the group consisting of cannabidiol (CBD), cannabichromene (CBC), cannabigerol (CBG) tetrahydrocannabinol (THC), tetrahydrocannabinolic acid (THCA) and cannabinol (CBN), and at least one cannabinoid selected from the group consisting of c-Eudesmol, Longifolene, Agarospirol, Neoisolongifolene, Ledol, Germacrene D, c-Maaliene, bisabolene, caryophyllene oxide, Longifolenaldehyde, and β-caryophyllene,

wherein a concentration of the THC, the THCA, the CBN, the β-caryophyllene, the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and/or the Longifolenaldehyde,

in the composition is 14-43% THC, 13-40% THCA, 0.5-1.5% CBN, 0-0.05 β-caryophyllene, 0.5-2.2% CBC, 2.5-12% CBD, 1-5% CBG, 0.1-0.5% c-Eudesmol, 0.4-2% Longifolene, 0.3-1.7% Agarospirol, 0.4-2% Neoisolongifolene, 0.1-0.6% Ledol, 0.5-2.2% e Germacrene D, 0.1-0.5% c-Maaliene, 0.2-0.9% bisabolene, 0.05-0.3% caryophyllene oxide, 0.05-0.4% Longifolenaldehyde.

According to some embodiments of the invention, a concentration of the THC, the THCA, the CBN, the β-caryophyllene, the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and the Longifolenaldehyde in the composition is as listed in Table 10.

According to some embodiments of the invention, the composition comprises a liquid chromatography fraction of cannabis extract.

According to some embodiments of the invention, the composition is a synthetic composition.

According to some embodiments of the invention, the cannabinoids are purified from cannabis.

According to some embodiments of the invention, the liquid chromatography fraction of cannabis extract comprises a liquid chromatography pooled fractions of cannabis extract comprising active ingredients detectable by a detector operated at 220 nm, wherein the active ingredients comprise the compounds of the (i), the (ii), the (iii) and/or the (iv).

According to some embodiments of the invention, the liquid chromatography comprises high pressure liquid chromatography (HPLC).

According to some embodiments of the invention, the liquid chromatography fraction or the composition comprises a liquid chromatography-purified cannabis extract obtainable by subjecting the cannabis extract to high pressure liquid chromatography (HPLC) and collecting fractions detectable by a detector operated at 220 nm,

wherein the HPLC of the (i) comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 21.91-26.42 minutes;

the HPLC of the (ii) comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 7.62-8.34 minutes;

the HPLC of the (iii) comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 24.24-25.07 minutes; and/or

the HPLC of the (iv) comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 22.88-24.23 minutes.

According to some embodiments of the invention, the liquid chromatography fraction or the composition has a cytotoxic activity on cutaneous T cell lymphoma (CTCL) cells.

According to some embodiments of the invention, the composition has a combined synergistic cytotoxic activity on cutaneous T cell lymphoma cells (CTCL) as compared to each of the cannabinoids when administered as a single agent.

According to an aspect of some embodiments of the present invention there is provided a method of generating a cytotoxic composition, the method comprising:

(a) adding a polar solvent to a Cannabis inflorescence so as to obtain a crude extract;

(b) filtering the crude extract so as to obtain a filtered extract;

(c) fractionating the filtered extract on a high pressure liquid chromatography (HPLC);

(d) collecting the liquid chromatography fractions detectable by a detector operated at 220 nm and comprising at least one fraction selected from the group consisting of

(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein the liquid chromatography fraction comprises at least 50% CBG and/or wherein the fraction is devoid of tetrahydrocannabinolic acid (THCA), and wherein the liquid chromatography fraction comprises the CBD the liquid chromatography fraction comprises at least two of the cannabinoids; and

(iv) a liquid chromatography fraction comprising cannabidiol (CBD), wherein the CBD is at least 90% CBD and/or wherein the liquid chromatography fraction is devoid of tetrahydrocannabinolic acid (THCA).

According to some embodiments of the invention, the HPLC of the (i) comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 21.91-26.42 minutes;

the HPLC of the (ii) comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 7.62-8.34 minutes;

the HPLC of the (iii) comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 24.24-25.07 minutes; and/or

the HPLC of the (iv) comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 22.88-24.23 minutes.

According to an aspect of some embodiments of the present invention there is provided a method of generating a cytotoxic composition, the method comprising:

(a) adding a polar solvent to a Cannabis inflorescence so as to obtain a crude extract;

(b) filtering the crude extract so as to obtain a filtered extract;

(c) fractionating the filtered extract on a high pressure liquid chromatography (HPLC);

(d) collecting the liquid chromatography fractions detectable by a detector operated at 220 nm and comprising at least one fraction selected from the group consisting of

(i) a fraction obtained at retention time 21.91-26.42 minutes, wherein the HPLC comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min;

(ii) a liquid chromatography fraction obtained at retention time 7.62-8.34 minutes, wherein the HPLC comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min;

(iii) a liquid chromatography fraction obtained at retention time 24.24-25.07 minutes, wherein the HPLC comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min; and

(iv) a liquid chromatography fraction obtained at retention time 22.88-24.23 minutes, wherein the HPLC comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min.

According to some embodiments of the invention, the liquid chromatography fraction of (i) comprises 40-60% tetrahydrocannabinol (THC) and/or 35-55% tetrahydrocannabinolic acid (THCA); the liquid chromatography fraction of (ii) comprises at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; the liquid chromatography fraction of (iii) comprises least one cannabinoid selected from the group consisting of cannabidiol (CBD), cannabichromene (CBC), tetrahydrocannabinol (THC) and cannabigerol (CBG), wherein the liquid chromatography fraction comprises at least 50% CBG and/or wherein the liquid chromatography fraction is devoid of tetrahydrocannabinolic acid (THCA); and/or the liquid chromatography fraction of (iv) comprises cannabidiol (CBD).

According to some embodiments of the invention, conditions for the HPLC comprise an Agilent Technologies 1260 Infinity preparative HPLC system coupled with a G1364B fraction collector-1260, a G1361A 1260 prep pump and a G1365D 1260 MWD-VL detector.

According to some embodiments of the invention, the C18 end caped column is a Kinetex 5 μm EVO C18 100A, 250×21.2 mm column.

According to an aspect of some embodiments of the present invention there is provided a cytotoxic composition obtainable by the method.

According to an aspect of some embodiments of the present invention there is provided a method of treating an inflammatory disease in a subject in need thereof, the method comprising administering to the subject a therapeutically effective amount of the composition or the article of manufacture, thereby treating the inflammatory disease in the subject.

According to some embodiments of the invention, the inflammatory disease is cancer.

According to some embodiments of the invention, the cancer is lymphoma.

According to some embodiments of the invention, the lymphoma is cutaneous T cell lymphoma (CTCL).

According to some embodiments of the invention, the liquid chromatography fraction or the composition of (iv) is devoid of tetrahydrocannabinolic acid (THCA).

According to some embodiments of the invention, the liquid chromatography fraction or the composition of (iv) comprises at least 90% CBD.

According to some embodiments of the invention, the liquid chromatography fraction or the composition of (iv) is devoid of cannabis derived active ingredients other than the CBD.

According to some embodiments of the invention, the liquid chromatography fraction or the composition of (iii) comprises at least two of the cannabinoids.

According to some embodiments of the invention, the liquid chromatography fraction or the composition of (iii) comprises all of the cannabinoids.

According to some embodiments of the invention, the liquid chromatography fraction or the composition of (iii) is devoid of cannabigerolic acid (CBGA) and/or cannabidiolic acid (CBDA).

According to some embodiments of the invention, the liquid chromatography fraction or the composition of (iii) comprises less than 2% CBC.

According to some embodiments of the invention, the liquid chromatography fraction or the composition of (iii) comprises less than 2% THC.

According to some embodiments of the invention, the liquid chromatography fraction or the composition of (iii) comprises at least 30% CBD.

According to some embodiments of the invention, the liquid chromatography fraction or the composition of (iii) comprises at least 50% CBG.

According to some embodiments of the invention, the liquid chromatography fraction or the composition of (iii) comprises at least 30% CBD, less than 2% CBC, less than 2% THC and at least 50% CBG.

According to some embodiments of the invention, the liquid chromatography fraction or the composition of (iii) is devoid of cannabis derived active ingredients other than the cannabinoids.

According to some embodiments of the invention, the liquid chromatography fraction or the composition of (iii) comprises about 38.25% CBD, about 0.44% CBC, about 0.74% THC and about 58.85% CBG.

According to some embodiments of the invention, the liquid chromatography fraction or the composition of (ii) comprises all of the CBC, the CBD and the CBG.

According to some embodiments of the invention, the liquid chromatography fraction or the composition of (ii) is devoid of tetrahydrocannabinolic acid (THCA).

According to some embodiments of the invention, the liquid chromatography fraction or the composition of (ii) is devoid of cannabigerolic acid (CBGA) and/or cannabidiolic acid (CBDA).

According to some embodiments of the invention, the liquid chromatography fraction or the composition of (ii) comprises all of the c-Eudesmol, the Longifolene, the Agarospirol, the Neoisolongifolene, the Ledol, the Germacrene D, the c-Maaliene, the bisabolene, the caryophyllene oxide and the Longifolenaldehyde.

According to some embodiments of the invention, the liquid chromatography fraction or the composition of (ii) is devoid of cannabis derived active ingredients other than the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and the Longifolenaldehyde.

According to some embodiments of the invention, the liquid chromatography fraction or the composition of (ii) comprises the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and the Longifolenaldehyde, in a concentration as listed in Table 8±10%.

According to some embodiments of the invention, the composition of (ii) comprises the CBC, the CBD and/or the CBG, in a concentration as listed in Table 8±10%.

According to some embodiments of the invention, the liquid chromatography fraction or the composition of (i) comprises about 50% THC.

According to some embodiments of the invention, the liquid chromatography fraction or the composition of (i) comprises tetrahydrocannabinolic acid (THCA).

According to some embodiments of the invention, the liquid chromatography fraction or the composition of (i) comprises 35-55% THCA.

According to some embodiments of the invention, the liquid chromatography fraction or the composition of (i) comprises 45-50% THCA.

According to some embodiments of the invention, the liquid chromatography fraction or the composition of (i) comprises cannabinol (CBN).

According to some embodiments of the invention, the liquid chromatography fraction or the composition of (i) comprises at least 1% CBN.

According to some embodiments of the invention, the liquid chromatography fraction or the composition of (i) comprises less than 3% CBN.

According to some embodiments of the invention, the liquid chromatography fraction or the composition of (i) comprises β-caryophyllene.

According to some embodiments of the invention, the liquid chromatography fraction or the composition of (i) comprises all of the THCA, the CBN and the β-caryophyllene.

According to some embodiments of the invention, the liquid chromatography fraction or the composition of (i) is devoid of at least one cannabinoid selected from the group consisting of cannabigerol (CBG), cannabidiol (CBD), cannabidiolic acid (CBDA), cannabigerolic acid (CBGA) and cannabichromene (CBC).

According to some embodiments of the invention, the liquid chromatography fraction or the composition of (i) is devoid of cannabidiol (CBD).

According to some embodiments of the invention, the liquid chromatography fraction or the composition of (i) is devoid of cannabigerolic acid (CBGA) and cannabichromene (CBC).

According to some embodiments of the invention, the liquid chromatography fraction or the composition of (i) is devoid of cannabidiol (CBD), cannabigerolic acid (CBGA) and cannabichromene (CBC).

According to some embodiments of the invention, the liquid chromatography fraction or the composition of (i) is devoid of cannabis derived active ingredients other than the THC, the THCA, the CBN and the β-caryophyllene.

According to some embodiments of the invention, the liquid chromatography fraction or the composition of (i) comprises the THC, the THCA, the CBN and/or the β-caryophyllene in a concentration as listed in Table 9±10%.

According to some embodiments of the invention, the liquid chromatography fraction or the composition comprises at least two of the liquid chromatography fractions or the compositions.

According to some embodiments of the invention, the at least two of the liquid chromatography fractions or the compositions comprise the (iii) and the (iv).

According to some embodiments of the invention, a total concentration of the CBD, the CBC, the THC and/or the CBG in the at least two of the liquid chromatography fractions or the compositions is 50-90% CBD, 10-40% CBG, 0.15-0.5% THC and/or 0.09-0.3% CBC.

According to some embodiments of the invention, a total concentration of the CBD, the CBC, the THC and/or the CBG in the at least two of the liquid chromatography fractions or the compositions is as listed in Table 5.

According to some embodiments of the invention, a molar ratio of the CBC to the CBD, a molar ratio of the THC to CBD and/or a molar ratio of the CBG to the CBD in the at least two of the compositions is as listed in Table 5.

According to some embodiments of the invention, the at least two of the liquid chromatography fractions or the compositions comprise the (i) and the (ii).

According to some embodiments of the invention, a total concentration of the THC, the THCA, the CBN, the β-caryophyllene, the CBC, the CBD and/or the CBG, in the at least two of the compositions is 14-43% THC, 13-40% THCA, 0.5-1.5% CBN, 0-0.05% β-caryophyllene, 0.5-2.2% CBC, 2.5-12% CBD and/or 1-5% CBG.

According to some embodiments of the invention, a total concentration of the THC, the THCA, the CBN, the β-caryophyllene, the CBC, the CBD and/or the CBG, in the at least two of the compositions is as listed in Table 10.

According to some embodiments of the invention, a total concentration of the THC, the THCA, the CBN, the β-caryophyllene, the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and/or the Longifolenaldehyde, in the at least two of the liquid chromatography fractions or the compositions is 14-43% THC, 13-40% THCA, 0.5-1.5% CBN, 0-0.05% β-caryophyllene, 0.5-2.2% CBC, 2.5-12% CBD, 1-5% CBG, the 0.1-0.5% c-Eudesmol, 0.4-2% Longifolene, 0.3-1.7% Agarospirol, 0.4-2% Neoisolongifolene, 0.1-0.6% Ledol, 0.5-2.2% Germacrene D, 0.1-0.5% c-Maaliene, 0.2-0.9% bisabolene, 0.05-0.3% caryophyllene oxide and/or 0.05-0.4% Longifolenaldehyde.

According to some embodiments of the invention, a total concentration of the THC, the THCA, the CBN, the β-caryophyllene, the CBC, the CBD, the CBG the c-Eudesmol, the Longifolene, the Agarospirol, the Neoisolongifolene, the Ledol, the Germacrene D, the c-Maaliene, the bisabolene, the caryophyllene oxide and the Longifolenaldehyde, in the at least two of the liquid chromatography fractions or the compositions is as listed in Table 10.

According to some embodiments of the invention, the at least two of the liquid chromatography fractions or the compositions have a combined synergistic cytotoxic activity on cutaneous T cell lymphoma cells (CTCL) as compared to each of the liquid chromatography fractions or the compositions.

According to some embodiments of the invention, the at least two of the liquid chromatography fractions or the compositions are provided in separate formulation.

According to some embodiments of the invention, the at least two of the liquid chromatography fractions or the compositions are provided in a co-formulation.

According to some embodiments of the invention, a concentration of the CBD, the CBC, the THC and/or the CBG in the composition of (iii) is 50-90% CBD, 10-40% CBG, 0.15-0.5 THC and/or 0.09-0.3% CBC.

According to some embodiments of the invention, a concentration of the CBD, the CBC, the THC and/or the CBG in the composition of (iii) is as listed in Table 5.

According to some embodiments of the invention, a concentration of the CBD, the CBC, the THC and/or the CBG in the liquid chromatography fraction or the composition of (iii) or (iv) is 50-90% CBD, 10-40% CBG, 0.15-0.5 THC and/or 0.09-0.3% CBC.

According to some embodiments of the invention, a concentration of the CBD, the CBC, the THC and/or the CBG in the liquid chromatography fraction or the composition of (iii) or (iv) is as listed in Table 5.

According to some embodiments of the invention, the method comprising ex-vivo contacting cutaneous T cells lymphoma (CTCL) cells of a subject with the composition, wherein a decrease in viability of the CTCL cells above a predetermined threshold as compared to same in the absence of the composition is indicative of the cytotoxic activity of the composition.

According to an aspect of some embodiments of the present invention there is provided a method of determining responsiveness of a subject having cutaneous T cell lymphoma (CTCL) to a cannabis-derived medicament, the method comprising:

(a) ex-vivo contacting CTCL cells of the subject with the cannabis-derived medicament;

(b) determining viability of the CTCL cells following the (a), wherein a decrease in viability above a predetermined threshold as compared to same in the absence of the cannabis-derived medicament is indicative of responsiveness to the cannabis-derived medicament.

According to some embodiments of the invention, the cannabis-derived medicament is a cannabis extract.

According to some embodiments of the invention, the cannabis-derived medicament is purified from cannabis.

According to some embodiments of the invention, the cannabis-derived medicament is a liquid chromatography fraction of a cannabis extract.

According to some embodiments of the invention, cannabis-derived medicament comprises the composition.

According to some embodiments of the invention, the cannabis-derived medicament is synthetic.

According to an aspect of some embodiments of the present invention there is provided a method of treating cutaneous T cell lymphoma (CTCL) in a subject in need thereof, the method comprising:

(a) ex-vivo contacting CTCL cells of the subject with at least one liquid chromatography fraction of a cannabis extract;

(b) determining viability of the CTCL cells following the (a); and

(c) administering to the subject a therapeutically effective amount of a liquid chromatography fraction or a combination of liquid chromatography fractions that induced a decrease in viability above a predetermined threshold as compared to same in the absence of the liquid chromatography fraction or the combination of liquid chromatography fractions,

thereby treating the CTCL in the subject.

According to an aspect of some embodiments of the present invention there is provided a method of treating cutaneous T cell lymphoma (CTCL) in a subject in need thereof, the method comprising:

(a) ex-vivo contacting CTCL cells of the subject with at least one liquid chromatography fraction of a cannabis extract;

(b) determining viability of the CTCL cells following the (a); and

(c) selecting for treatment a liquid chromatography fraction or a combination of liquid chromatography fractions that induced a decrease in viability above a predetermined threshold as compared to same in the absence of the liquid chromatography fraction or the combination of liquid chromatography fractions,

thereby treating the CTCL in the subject.

According to some embodiments of the invention, the at least one liquid chromatography fraction of a cannabis extract comprises the composition.

According to some embodiments of the invention, the cannabis is a cannabis strain SCBD.

According to some embodiments of the invention, the cannabis is a cannabis strain DQ.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 depicts the dose-effect curve of C. sativa strain SCBD extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIG. 2 depicts the effect of different HPLC separated fractions of C. sativa strain SCBD extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIGS. 3A-B depict the dose-effect curve (FIG. 3A) and the IC50 (FIG. 3B) of fraction S4 separated from C. sativa strain SCBD extracts on viability of MyLa cancer cells following 48 hours of treatment, as determined by XTT assay. IC50=16.09 μg/ml. Bars represent mean±SD, n=3. Levels with different letters are significantly different from all combinations of pairs by Tukey-Kramer honest significant difference (HSD).

FIGS. 4A-B depict the dose-effect curve (FIG. 4A) and the IC50 (FIG. 4B) of fraction S5 separated from C. sativa strain SCBD extracts on viability of MyLa cancer cells following 48 hours of treatment, as determined by XTT assay. IC50=9.72 μg/ml. Bars represent mean±SD, n=3. Levels with different letters are significantly different from all combinations of pairs by Tukey-Kramer honest significant difference (HSD).

FIGS. 5A-C depict the cytotoxic effect (FIG. 5A) and the IC50 (FIGS. 5B-C) of combination of fractions S4 and S5 separated from C. sativa strain SCBD extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIGS. 6A-B depict the effect of combination of fractions S4 and S5 separated from C. sativa strain SCBD extracts on viability of HUT78 cancer cells following 48 hours of treatment, as determined by XTT assay. Bars represent mean±SD, n=3. Levels with different letters are significantly different from all combinations of pairs by Tukey-Kramer honest significant difference (HSD).

FIG. 7 depicts the GC/MS profile of fraction S4 separated from C. sativa strain SCBD extracts.

FIG. 8 depicts the GC/MS profile of fraction S5 separated from C. sativa strain SCBD extracts.

FIG. 9 depicts the dose-effect curve of C. sativa strain DQ extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIG. 10 depicts the effect of different HPLC separated fractions of C. sativa strain DQ extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIGS. 11A-B depict the dose-effect curve (FIG. 11A) and the IC50 (FIG. 11B) of fraction D2 separated from C. sativa strain DQ extracts on viability of MyLa cancer cells following 48 hours of treatment, as determined by XTT assay. IC50=41.3 μg/ml. Bars represent mean±SD, n=3. Levels with different letters are significantly different from all combinations of pairs by Tukey-Kramer honest significant difference (HSD).

FIGS. 12A-B depict the dose-effect curve (FIG. 12A) and the IC50 (FIG. 12B) of fraction D6 separated from C. sativa strain DQ extracts on viability of MyLa cancer cells following 48 hours of treatment, as determined by XTT assay. IC50=17.4 μg/ml. Bars represent mean±SD, n=3. Levels with different letters are significantly different from all combinations of pairs by Tukey-Kramer honest significant difference (HSD).

FIG. 13 depicts the cytotoxic effect of combination of fractions D2 and D6 separated from C. sativa strain DQ extracts on viability of MyLa cancer cells following 48 hours of treatment, as determined by XTT assay. Bars represent mean±SD, n=3. Levels with different letters are significantly different from all combinations of pairs by Tukey-Kramer honest significant difference (HSD).

FIG. 14 depicts the GC/MS profile of fraction D2 separated from C. sativa strain DQ extracts.

FIG. 15 depicts the GC/MS profile of fraction D6 separated from C. sativa strain DQ extracts.

FIG. 16 depicts the HPLC chromatogram of C. sativa strain SCBD extracts.

FIG. 17 depicts the HPLC chromatogram of C. sativa strain DQ extracts.

FIG. 18 depicts IL-13 mRNA expression levels in MyLa cancer cells following 4 hours treatment with the indicated fractions separated from C. sativa strain SCBD extracts. NT—non-treated, MeOH—methanol control. Bars represent mean±SD, n=3. Levels with different letters are significantly different from all combinations of pairs by Tukey-Kramer honest significant difference (HSD).

FIG. 19 depicts the effect of pre-treatment with CB1 and CB2 receptors inverse agonists (IA CB1 and IA CB2) on the cytotoxic effect of combination of fractions S4 and S5 separated from C. sativa strain SCBD extracts on viability of MyLa cancer cells following 48 hours of treatment, as determined by XTT assay. NT—non-treated, MeOH—methanol control. Bars represent mean±SD, n=3. Levels with different letters are significantly different from all combinations of pairs by Tukey-Kramer honest significant difference (HSD). *—statistically significant compared to S4+S5 treatment without the inverse agonists, based on T test.

FIGS. 20A-F depict the effect of fractions S4 or S5 separated from C. sativa strain SCBD extracts or their combination on apoptosis and cell cycle arrest of My-La and HUT78 cancer cells. FIGS. 20A-B demonstrate stages of cell cycle arrest following treatment of My-La (FIG. 20A) or HuT-78 (FIG. 20B) cells. Starved cells were treated with S4 [5 μg/mL], S5 [6 μg/mL], S4 [5 μg/mL]+S5 [6 μg/mL] or methanol (control) for 48 hours. The treated cells were harvested, fixed, and analyzed in FACS following propidium iodide (PI) staining. The percentage of cells in G0/G1, G2/M and S phase were analyzed from 10,000 events per treatment. Bars represent % cell count ±SE (2 biological replicates, in each n=3). Levels with different letters are significantly different from all combinations of pairs according to Tukey-Kramer honest significant difference (HSD; P<0.05). FIGS. 20C-F demonstrate proportions of viable, apoptotic or necrotic cells following treatment of My-La (FIGS. 20C and 20E) or HuT-78 (FIGS. 20D and 20F) cells. Cells were treated with S4 [5 μg/mL], S5 [6 μg/mL], S4 [5 μg/mL]+S5 [6 μg/mL] or methanol (control) for 48 hours. The treated cells were harvested and analyzed in FACS following Annexin V-FITC and PI staining. FIGS. 20C-D show the percentages of viable, necrotic, and apoptotic cells, analyzed from 10,000 cells per treatment Bars represent % cell count ±SE (2 biological replicates, in each n=3). Levels with different letters are significantly different from all combinations of pairs according to Tukey-Kramer honest significant difference (HSD; P<0.05). FIGS. 20E-F show representative scatter plots of the FACS analysis. The scatter plot for each sample was split into four quadrants to indicate viable cells (lower left quadrant, Q4), early apoptotic cells (lower right quadrant, Q3), necrotic cells (upper left quadrant, Q1), and late apoptotic cells (upper right quadrant, Q2).

FIGS. 21A-C depict the effect of fractions S4 or S5 separated from C. sativa strain SCBD extracts or their combination on viability and apoptosis of PBLs of Sézary patients. PBL were isolated from blood samples of Sézary patients, and were treated for 48 hours with S4 [5 μg/mL], S5 [6 μg/mL], S4 [5 μg/mL]+S5 [6 μg/mL] or methanol (control). Following, cells were harvested and analyzed by FACS following CD4-APC, CD26-alexa 405, Annexin V-FITC and PI staining. Apoptotic-induced cells (Annexin positive cells) were determined in the CD4⁺CD26⁻ cell population and in non-CD4⁺CD26⁻ cells of treated cells minus control. FIG. 21A shows representative scatter plots of the FACS analysis. FIG. 21B is a bar graph demonstrating the percent of apoptotic-induced CD4⁺CD26⁻ cells. Bars represent percent of apoptotic-induced CD4⁺CD26⁻±SE (n=4); *** denotes significant difference between means (one way ANOVA; p<0.001). FIG. 21C is a bar graph demonstrating percent of apoptotic-induced cells in CD4⁺CD26⁻ cells as compared to non-CD4⁺CD26⁻ cells. Bars represent percent of apoptotic-induced cells±SE (n=6); ** denotes significant difference between means (paired student T test; 0.001<P<0.05).

FIGS. 22A-B depict hierarchical clustering (FIG. 22A) and Venn diagram (FIG. 22B) of genes significantly differentially expressed genes in My-La and HuT-78 cells treated with S4, S5 or their combination. FIG. 22A shows hierarchical clustering using Pearson correlations among the four conditions based on the genes expression (average fragments per kilobase of transcript per million fragments mapped [FPKM] of the three replications) followed by a log 2 transform. Pearson correlations were calculated with the R software. FIG. 22B shows Venn diagrams illustrating the relationships between significantly differentially expressed genes (padj<0.05) in the three treatments against the control. Venn diagrams were generated using the online tool at bioinformatics(dot)psb(dot)ugent(dot)be/webtools/Venn/.

FIGS. 23A-B depict the cytotoxic effect of pure CBD, CBG, THC and CBC cannabinoids at concentrations found in a combination of S4+S5 fractions on viability of MyLa cancer cells (FIG. 23A) and Hut-78 cells (FIG. 23B) following 48 hours of treatment, as determined by XTT assay. Specifically, cells were treated with S4 [5 μg/mL], S5 [6 μg/mL], CBD [7.3 μg/ml], CBG [3.5 μg/ml], THC [0.044 μg/ml] and CBC [0.027 μg/ml], and the indicated combinations. Methanol treatment served as solvent control; and Doxorubicin (DOXO, 300 nM) served as a positive control for cell cytotoxicity. Bars represent mean percent of viable cells±SE, n=3. Levels with different letters are significantly different from all combinations of pairs treated by a certain S4+S5 combinations or untreated by Tukey-Kramer honest significant difference (HSD, P≤0.05).

### DESCRIPTION OF SPECIFIC EMBODIMENTS OF THE INVENTION

The present invention, in some embodiments thereof, relates to compositions and methods for the treatment of cutaneous T cell lymphoma.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Cutaneous T-cell lymphomas (CTCL) are the most frequent primary lymphomas of the skin. Marijuana (Cannabis sativa) contains more than 500 constituents, among them more than a hundred terpenophenolic compounds termed phytocannabinoids [ElSohly et al., Phytochemistry of Cannabis sativa L. Phytocannabinoids, Springer (2017) 1-36].

Different constituents and preparations of marijuana (Cannabis sativa) have been shown to have beneficial effects on proliferation, metastasis, angiogenesis and induction of apoptosis in a variety of cancer cell types. However, C. sativa contains hundreds of compounds and very little is known about their effect on cancerous cells, in general, and CTCL cells, in particular.

The present inventor has now uncovered that liquid chromatography fractions of cannabis inflorescence extracts are effective as cytotoxic agents and can efficiently eradicate CTCL cells.

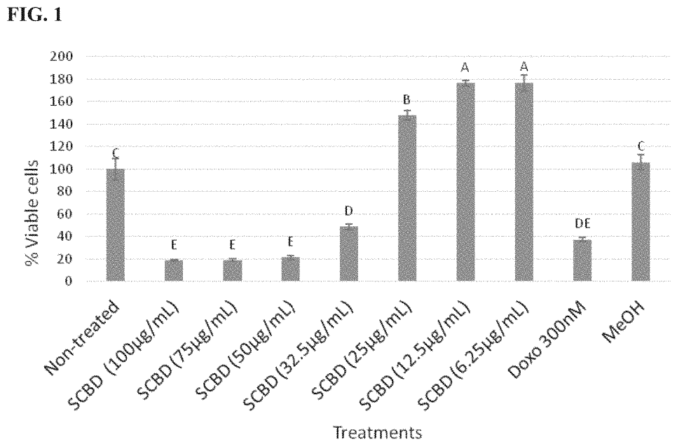

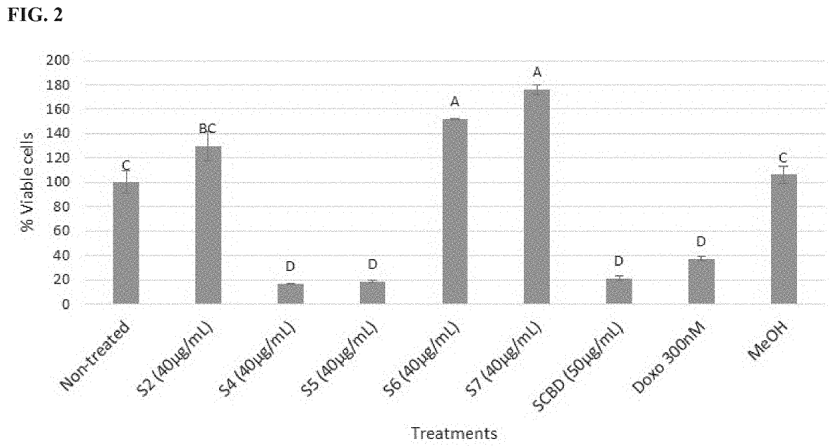

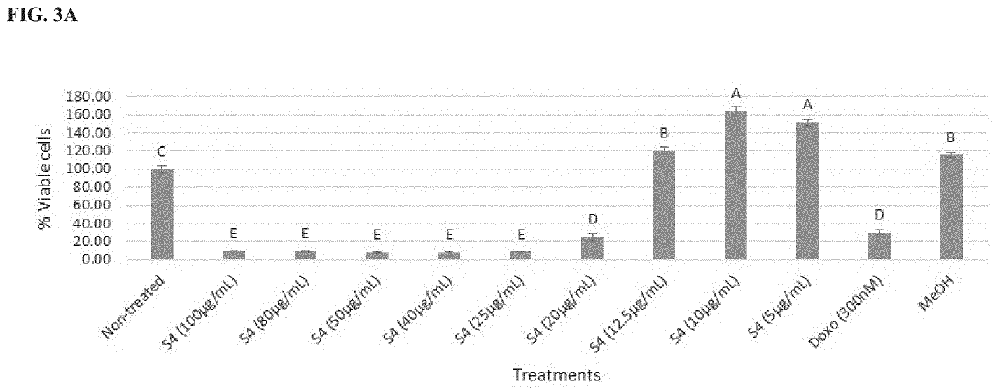

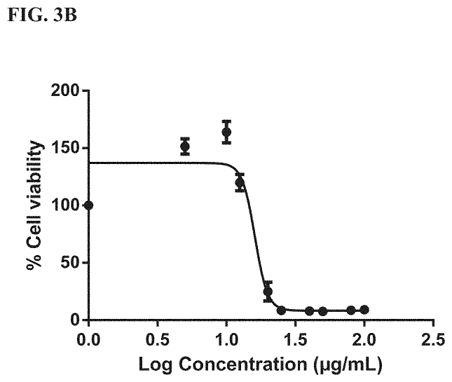

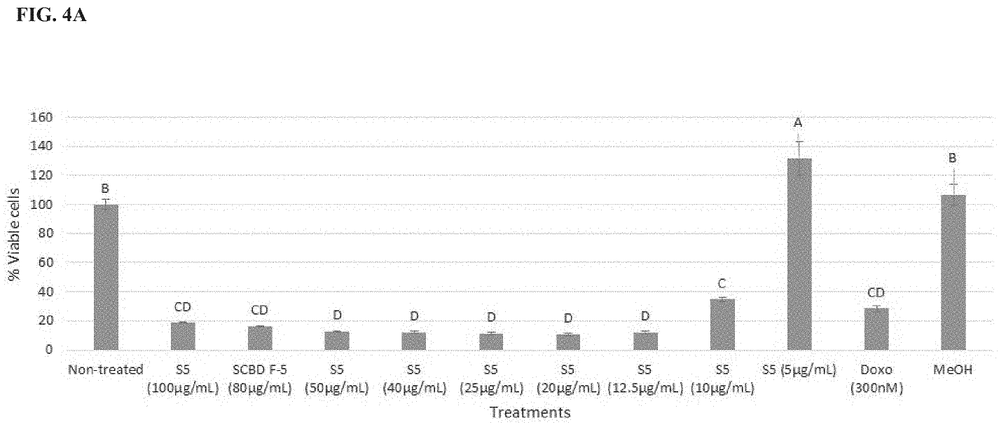

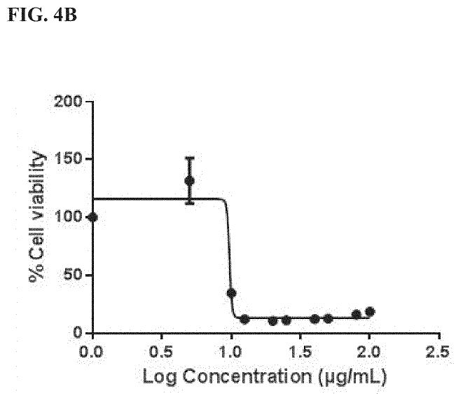

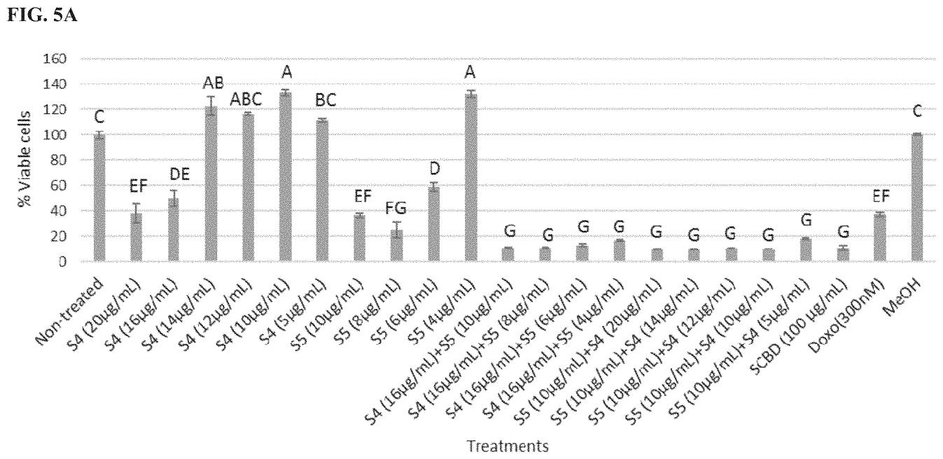

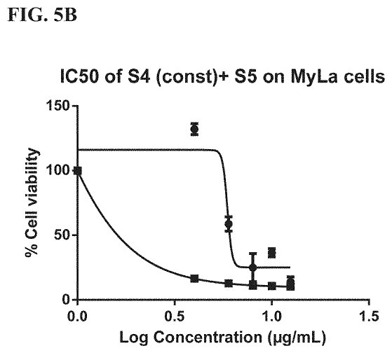

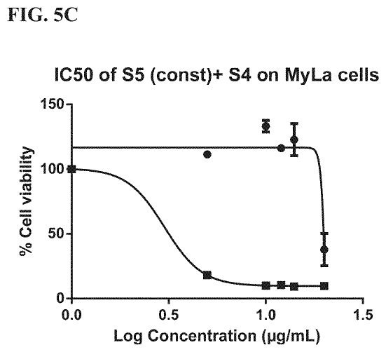

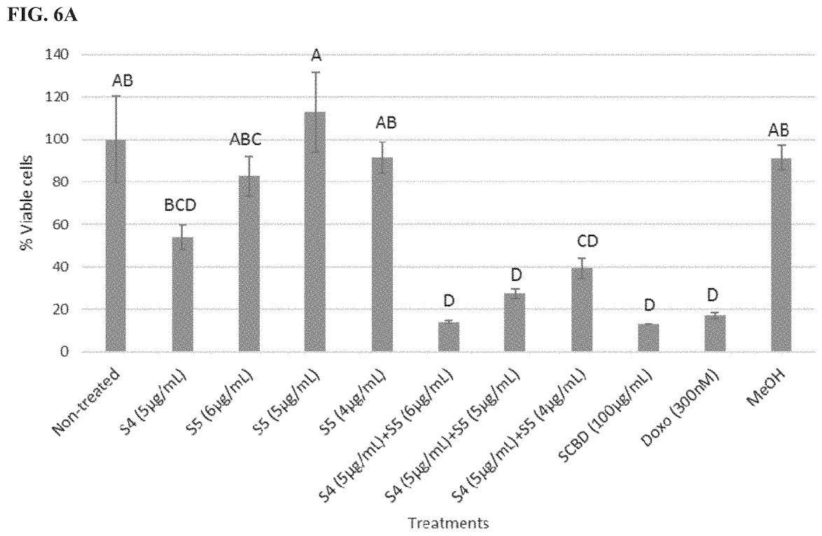

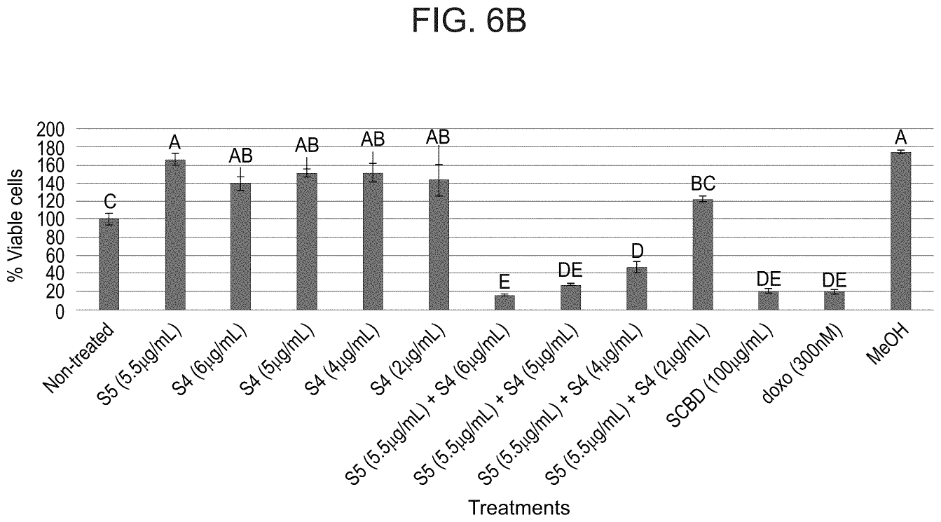

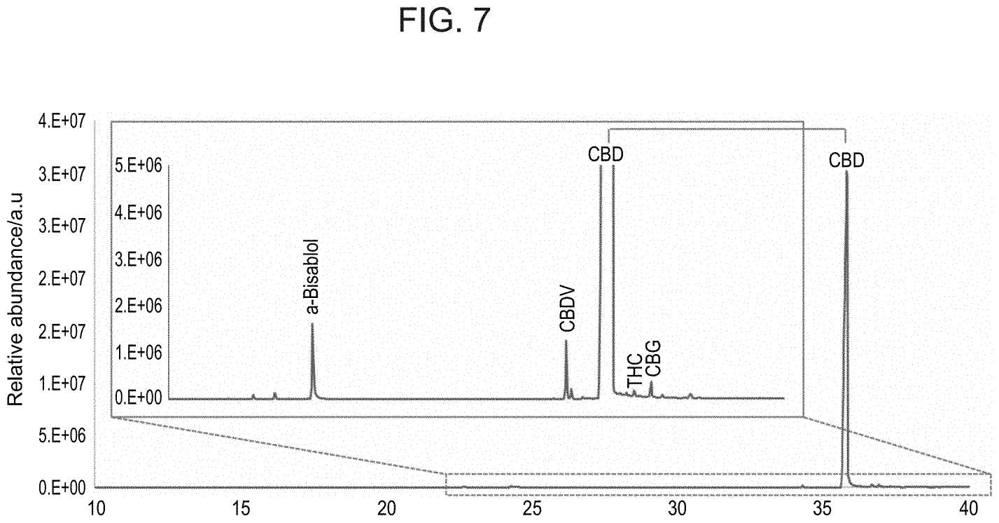

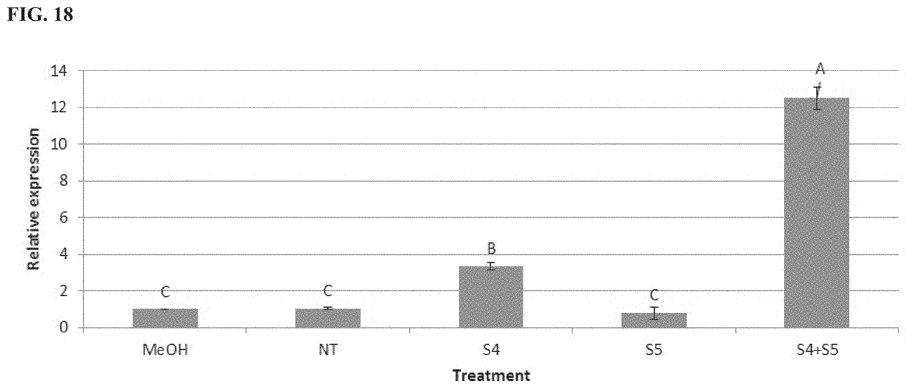

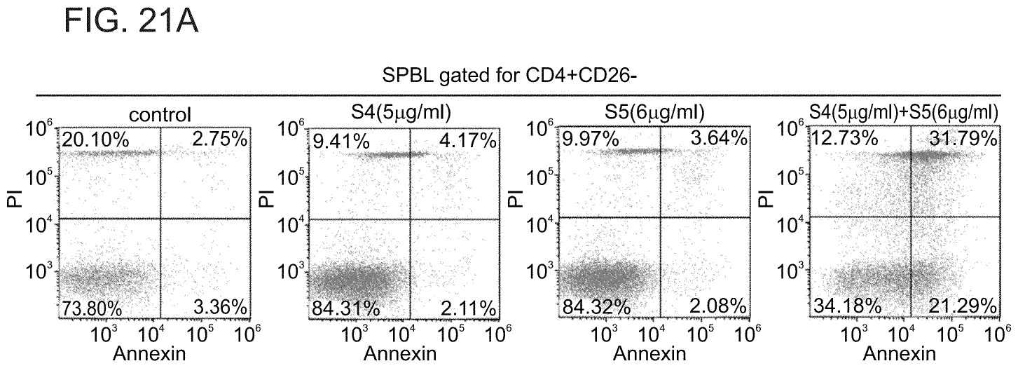

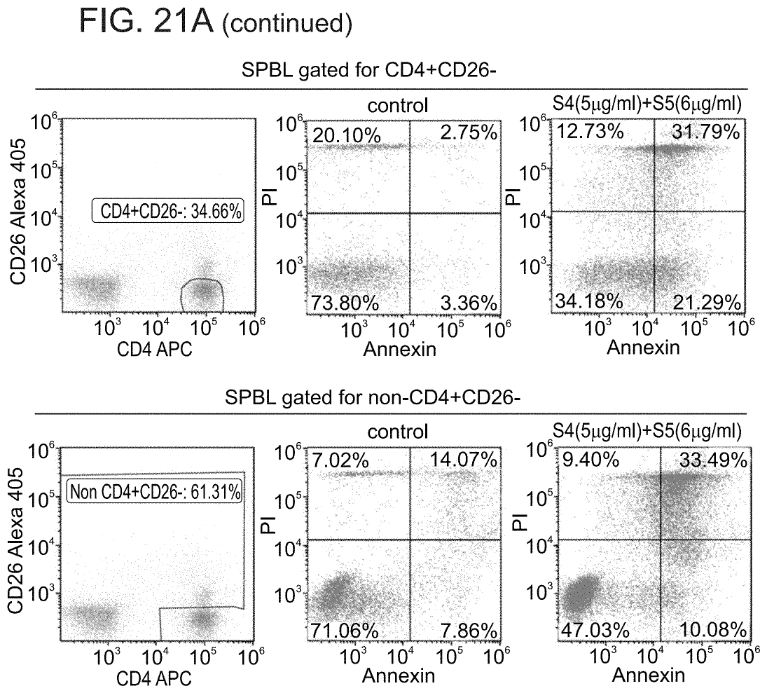

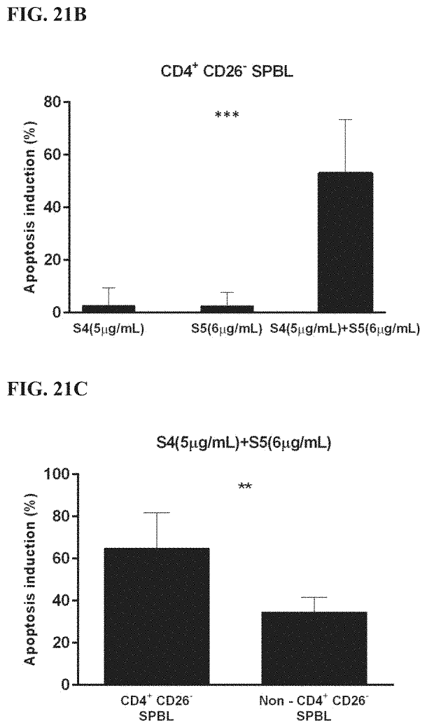

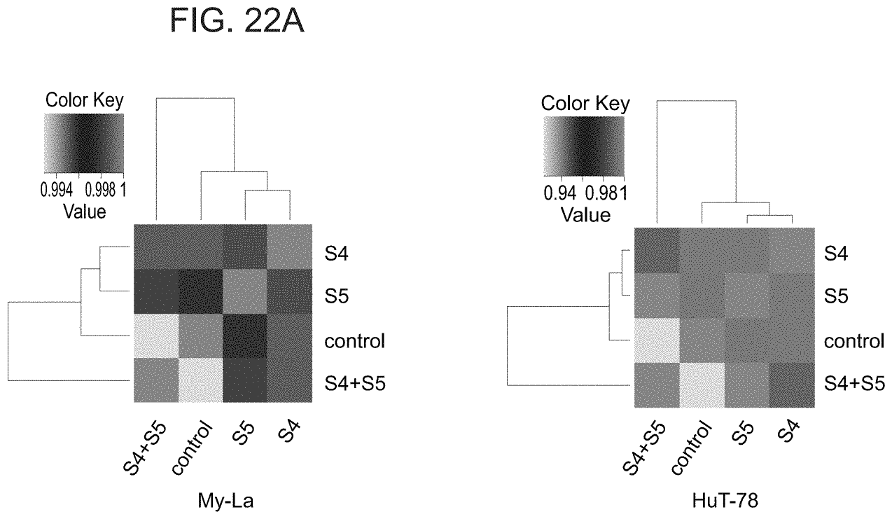

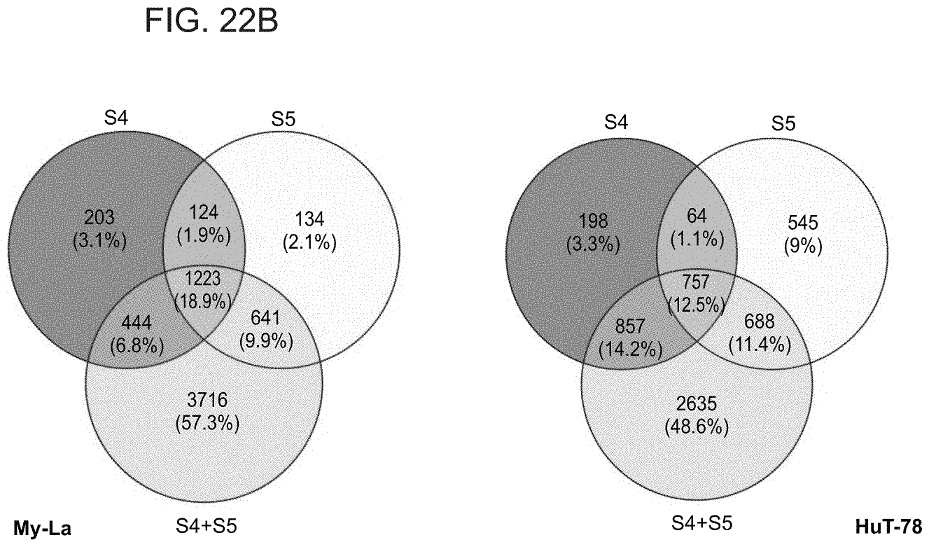

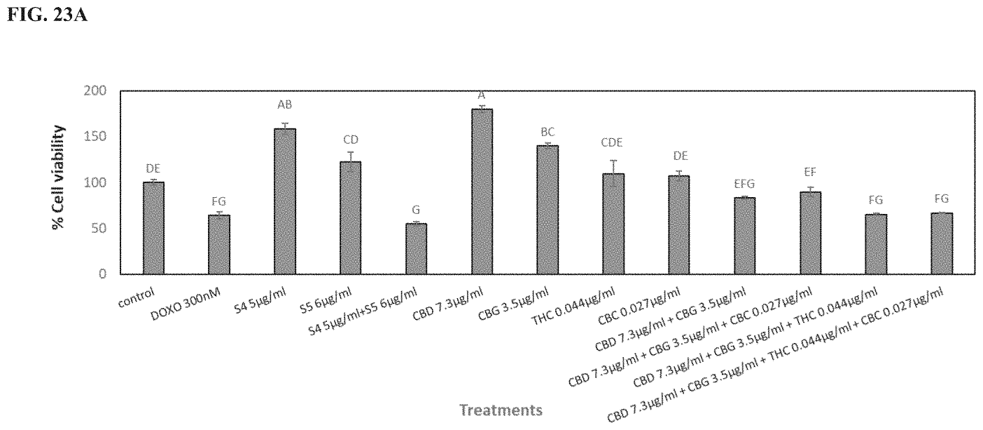

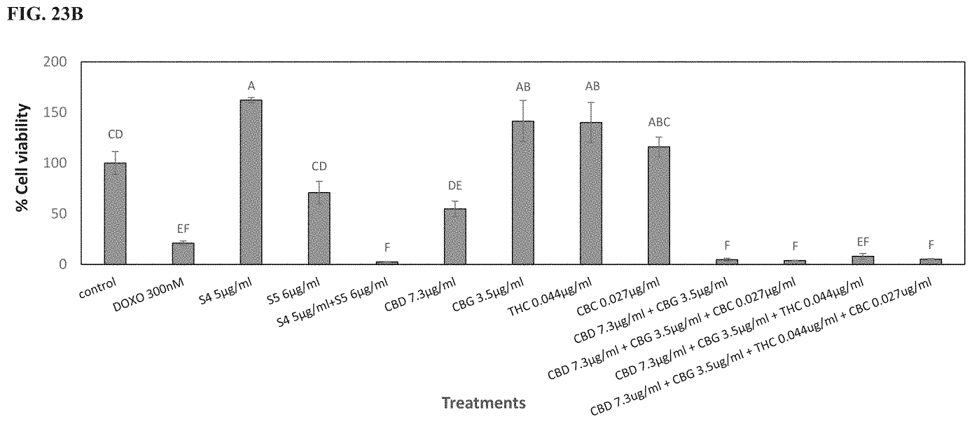

Specifically, the present inventors obtained fresh inflorescences of C. sativa strain DQ and dry inflorescences of C. sativa strain SCBD which exhibited specific cytotoxic activity towards CTCL cell lines [namely, MyLa cells (lymphoma cells established from skin biopsies of a patient with Mycosis Fungoides) and HUT78 cells (lymphoma cells established from PBL of a patient with Sezary Syndrome)] (Examples 1 and 3 of the Examples section which follows, FIGS. 1, 6A-B and 9). These extracts were further fractionated into several distinct fractions among them fractions which illustrated specific cytotoxic activity, i.e. fractions S4 and S5 from C. sativa strain SCBD and fractions D2 and D6 from C. sativa strain DQ (Examples 1 and 3 of the Examples section which follows, FIGS. 2, 3A-B, 4A-B, 10, 11A-B and 12A-B). In addition, the cytotoxic activity of the fractions was found to involve cell apoptosis and cell cycle arrest; treatment was accompanied by changes in gene expression such as increased levels of IL-13; and pre-treatment with inverse agonists of CB2 receptors reduced the fractions cytotoxic activity (Example 1 of the Examples section which follows, FIGS. 18-19, 20A-F and 22A-B). The effects of the obtained fractions and their combinations were also validated in-vitro on peripheral blood lymphocytes obtained from Sézary patients (SPBL) (Example 1 of the Examples section which follows, FIGS. 21A-C). The composition of each of these fractions is shown in FIGS. 7-8 and 14-15 and Tables 3-4 and 8-9 in the Examples section which follows. Furthermore, synergistic activity was evident when combining treatment with fractions S4 and S5 or with D2 and D6 (see Examples 1 and 3 of the Examples section which follows, FIGS. 5A-C, 6A-B, 13, 18-21C). In the next step the present inventors showed that combinations of pure cannabinoids at the concentrations present in the combined active fractions S4 and S5 composition had a synergistic cytotoxic activity similar to the S4 and S5 combined fractions (Example 2 of the Examples section which follows, FIGS. 22A-23B).

Consequently, specific embodiments of the present invention propose that these novel cannabis fractions and compositions comprising same may be used as therapeutic agents for inflammatory diseases in general and CTCL in particular.

Thus, according to one aspect of the present invention, there is provided a method of generating a cytotoxic composition, the method comprising:

(a) adding a polar solvent to a Cannabis inflorescence so as to obtain a crude extract;

(b) filtering the crude extract so as to obtain a filtered extract;

(c) fractionating the filtered extract on a high pressure liquid chromatography (HPLC);

(d) collecting the liquid chromatography fractions detectable by a detector operated at 220 nm and comprising at least one fraction selected from the group consisting of

(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least 50% CBG and/or wherein said fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising cannabidiol (CBD), wherein said CBD is at least 90% CBD and/or wherein said liquid chromatography fraction is devoid of tetrahydrocannabinolic acid (THCA).

According to an additional or alternative aspect of the present invention, there is provided a method of generating a cytotoxic composition, the method comprising:

(a) adding a polar solvent to a Cannabis inflorescence so as to obtain a crude extract;

(b) filtering the crude extract so as to obtain a filtered extract;

(c) fractionating the filtered extract on a high pressure liquid chromatography (HPLC);

(d) collecting the liquid chromatography fractions detectable by a detector operated at 220 nm and comprising at least one fraction selected from the group consisting of;

(i) a liquid chromatography fraction comprising 40-60% tetrahydrocannabinol (THC);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fractions is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least 50% CBG and/or wherein said fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising at least 90% cannabidiol (CBD) and at least one cannabinoid other than said CBD.

According to an additional or alternative aspect of the present invention, there is provided a method of generating a cytotoxic composition, the method comprising:

(a) adding a polar solvent to a Cannabis inflorescence so as to obtain a crude extract;

(b) filtering the crude extract so as to obtain a filtered extract;

(c) fractionating the filtered extract on a high pressure liquid chromatography (HPLC);

(d) collecting the liquid chromatography fractions detectable by a detector operated at 220 nm and comprising at least one fraction selected from the group consisting of

(i) a fraction obtained at retention time 21.91-26.42 minutes, wherein said HPLC comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min;

(ii) a liquid chromatography fraction obtained at retention time 7.62-8.34 minutes, wherein said HPLC comprises an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min;

(iii) a liquid chromatography fraction obtained at retention time 24.24-25.07 minutes, wherein said HPLC comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min; and

(iv) a liquid chromatography fraction obtained at retention time 22.88-24.23 minutes, wherein said HPLC comprises an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min.

Cannabis is a genus of flowering plants in the family Cannabaceae that includes three different species, Cannabis sativa, Cannabis indica and Cannabis ruderalis. The term Cannabis encompasses wild type Cannabis and also variants thereof, including cannabis chemovars which naturally contain different amounts of the individual cannabinoids. For example, some Cannabis strains have been selectively bred to produce high or low levels of THC and other cannabinoids.

According to specific embodiments, the Cannabis plant is a wild-type plant.

According to specific embodiments, the Cannabis plant is transgenic.

According to specific embodiments, the Cannabis plant is genomically edited.

According to specific embodiments, the Cannabis plant is Cannabis sativa (C. sativa).

According to specific embodiments, the Cannabis plant is C. sativa strain SCBD (obtained from IMC, Israeli Medical Cannabis, Israel)

According to specific embodiments, the Cannabis plant is C. sativa strain DQ (obtained from IMC, Israeli Medical Cannabis, Israel).

The extract may be derived from a cultivated Cannabis plant (i.e. not grown in their natural habitat) or may be derived from Cannabis plants which grow in the wild.

The tissue of the Cannabis plant from which the extract is typically obtained is the inflorescence. Accordingly, the extract may be obtained from the complete flower head of a plant including stems, stalks, bracts, and flowers. However, it will be appreciated that a cannabis extract of the invention may be obtained from only part of the inflorescence, such as from the bracts and/or flowers.

According to specific embodiments, the extract is obtained from a fresh plant (i.e. a plant not heated prior to the extraction process). Fresh plants include plants taken immediately following harvesting (e.g., up to an hour or several hours) for extraction as well as plants frozen immediately after harvesting (e.g. at about −70° C. to −90° C., e.g. at −80° C., for any required length of time) prior to extraction.

According to specific embodiments, the extract is obtained from fresh inflorescence.

According to specific embodiments, the extract is obtained from a frozen inflorescence (e.g. frozen immediately after harvesting at about −70° C. to −90° C., e.g. at −80° C., for any required length of time). Thus, for example, the extract may be obtained from a cryopreserved inflorescence, or from an inflorescence frozen in liquid nitrogen or in dry ice.

According to specific embodiments, the extract is obtained from an inflorescence which has not been subjected to heating (such as heating at e.g. at 120° C. to 180° C., e.g. at 150° C., for any length of time, such as for 1-5 hours).

According to specific embodiments, the extract is obtained from dry Cannabis inflorescence. Drying the inflorescence may be carried out using any method known in the art, such as by pulverizing with liquid nitrogen or with dry-ice/alcohol mixture.

According to specific embodiments, the dry inflorescence is obtained from the grower.

According to specific embodiments, the polar solvent comprises a polar, protic solvent (e.g., ethanol or methanol). In some embodiments, the polar solvent comprises a polar, aprotic solvent (e.g., acetone). Polar solvents suitable for use with specific embodiments of the present invention include, but are not limited to, ethanol, methanol, n-propanol, iso-propanol, a butanol, a pentanol, acetone, methylethylketone, ethylacetate, acetonitrile, tetrahydrofuran, dimethylformamide, dimethylsulfoxide, water, and combinations thereof.

According to specific embodiments, the polar solvent is ethanol (e.g. absolute ethanol i.e. above 99.8%, or in the range of 99-70% in water).

The concentration or amount of a polar solvent used to Cannabis inflorescence can be varied. Generally, the ratio of a Cannabis inflorescence to a polar solvent (weight to volume) is the amount of a polar solvent sufficient to extract about 70% or more, about 75% or more, about 85% or more, about 90% or more, about 95% or more, about 97% or more, or about 99% or more of a composition having a cytotoxic activity. In some embodiments, the ratio of polar solvent to Cannabis inflorescence is about 1:2 to about 1:20 (w/v), e.g. about 1:4 to about 1:10 (w/v).

In particular embodiments, the extract is an ethanol extract.

In particular embodiments, absolute ethanol is added to the inflorescence at a sample-to-absolute ethanol ratio of 1:4 (w/v).

In some embodiments, the Cannabis inflorescence is contacted with a polar solvent (e.g. ethanol) for about 15 minutes or more, about 30 minutes or more, about 1 hour or more, about 2 hours or more, or about 5 hours or more.

According to specific embodiments, the Cannabis inflorescence is contacted with a polar solvent (e.g. ethanol) for about 30 minutes.

Temperature can also be controlled during the contacting. In some embodiments, the Cannabis inflorescence is contacted with a polar solvent at temperature of about 15° C. to about 35° C., or about 20° C. to about 25° C.

According to specific embodiments, the Cannabis inflorescence is contacted with a polar solvent (e.g. ethanol) while being constantly mixed e.g. on a shaker.

In some embodiments, the process of the present invention comprises isolating a liquid extract (i.e. filtered extract) from the mixture (i.e. crude extract) comprising the liquid extract and solids. Suitable means for isolating the liquid extract (i.e. filtered extract) include those known in the art of organic synthesis and include, but are not limited to, gravity filtration, suction and/or vacuum filtration, centrifuging, setting and decanting, and the like. In some embodiments, the isolating comprises filtering a liquid extract through a porous membrane, syringe, sponge, zeolite, paper, or the like having a pore size of about 1-5 μm, about 0.5-5 μm, about 0.1-5 μm, about 1-2 μm, about 0.5-2 μm, about 0.1-2 μm, about 0.5-1 μm, about 0.1-1 μm, about 0.25-0.45 μm, or about 0.1-0.5 μm (e.g. about 2 μm, about 1 μm, about 0.45 μm, or about 0.25 μm).

According to a specific embodiment, the crude extract is filtered through a 0.45-μm syringe filter such as that commercially available from Merck, Darmstadt, Germany.

According specific embodiments, the present invention contemplates drying (i.e. removal of the polar solvent) and/or freezing the filtered extract following generation thereof.

The method for drying the filtered extract (i.e. removing the polar solvent) is not particularly limited, and can include solvent evaporation at a reduced pressure (e.g., sub-atmospheric pressure) and/or an elevated temperature (e.g., above about 25° C.). In some embodiments, it can be difficult to completely remove a polar solvent from a liquid extract by standard solvent removal procedures such as evaporation. In some embodiments, processes such as co-evaporation, lyophilization, and the like can be used to completely remove the polar solvent from a liquid fraction to form a dry powder, dry pellet, dry granulate, paste, and the like. According to a specific embodiment the polar solvent is evaporated with a vacuum evaporator.

Following generation of the filtered extract, specific embodiments of the present invention further contemplate additional purification steps so as to further purify active agents from the extract.

Thus, for example, fractionating the filtered extract. Fractionating can be performed by processes such as, but not limited to: column chromatography, preparative high performance liquid chromatography (“HPLC”), reduced pressure distillation, and combinations thereof. According to a specific embodiment, fractionating is performed by HPLC.

In some embodiments, fractionating comprises resuspending the filtered extract in a polar solvent (such as methanol, as discussed above), applying the polar extract to a separation column, and isolating the Cannabis extract having a cytotoxic activity by column chromatography (preparative HPLC).

An eluting solvent is applied to the separation column with the polar extract to elute fractions from the polar extract. Suitable eluting solvents for use include, but are not limited to, methanol, ethanol, propanol, acetone, acetic acid, carbon dioxide, methylethyl ketone, acetonitrile, butyronitrile, carbon dioxide, ethyl acetate, tetrahydrofuran, di-iso-propylether, ammonia, triethylamine, N,N-dimethylformamide, N,N-dimethylacetamide, and the like, and combinations thereof.

According to an alternative or an additional embodiment, liquid chromatography comprises high performance liquid chromatography (HPLC).

According to an alternative or an additional embodiment, liquid chromatography is performed on a reverse stationary phase.

According to an alternative or an additional embodiment, liquid chromatography is performed using a mobile phase comprising from 10 to 40% acidic aqueous solution and from 60 to 90% alcohol.

According to a specific embodiment, an eluting solvent comprises 15-40% solvent A (0.1% acetic acid in water) and 60-85% solvent B (methanol).

According to specific embodiments, fraction separation may be carried out on a HPLC comprising an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 21.91-26.42 minutes [e.g. for the fraction of (i)].

According to specific embodiments, fraction separation may be carried out on a HPLC comprising an Agilent Technologies 1260 Infinity preparative HPLC system coupled with a 1260 MWD-VL detector, a C18 end capped column and a mobile phase of 25% solvent A (0.1% acetic acid in water) and 75% solvent B (100% methanol) at a flow rate of 30 mL/min for about 7.62-8.34 minutes [e.g. for the fraction of (ii)].

According to specific embodiments, fraction separation may be carried out on a HPLC an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 24.24-25.07 minutes [e.g. for the fraction of (iii)].

According to specific embodiments, fraction separation may be carried out on a HPLC comprising an Agilent Technologies 1260 Infinity preparative HPLC system coupled with a 1260 MWD-VL detector, a C18 end capped column and a mobile phase of 40% solvent A (0.1% acetic acid in water) and 60% solvent B (100% methanol) at a flow rate of 30 mL/min for about 22.88-24.23 minutes [e.g. for the fraction of (iv)].

According to alternative or additional embodiments, fractions comprising components (active ingredients) are detectable by a detector operated at 220 nm are collected.

According to alternative or additional embodiments, the detector is a diode array detector.

According to alternative or additional embodiments, the detector is a 1260 MWD-VL detector.

According to specific embodiments, the conditions for HPLC comprise an Agilent Technologies 1260 Infinity preparative HPLC system coupled with a G1364B fraction collector-1260, a G1361A 1260 prep pump and a G1365D 1260 MWD-VL detector.

According to specific embodiments, the C18 end caped column is a Kinetex 5 μm EVO C18 100A, 250×21.2 mm column (available from Phenomenex).

According to specific embodiments, the conditions for HPLC comprise an Agilent Technologies 1260 Infinity preparative HPLC system coupled with a G1364B fraction collector-1260, a G1361A 1260 prep pump, a G1365D 1260 MWD-VL detector, a C18 end caped column (e.g. Kinetex 5 μm EVO C18 100A, 250×21.2 mm column) and a mobile phase of 25% solvent A (0.1% acetic acid in water) and 75% solvent B (100% methanol) at a flow rate of 30 mL/min for about 21.91-26.42 minutes [e.g. for the fraction of (i)].

According to specific embodiments, the conditions for HPLC comprise a 1260 Infinity preparative HPLC system coupled with an Agilent Technologies 1260 Infinity preparative HPLC system coupled with a G1364B fraction collector-1260, a G1361A 1260 prep pump, a G1365D 1260 MWD-VL detector, a C18 end caped column (e.g. Kinetex 5 μm EVO C18 100A, 250×21.2 mm column) and a mobile phase of 25% solvent A (0.1% acetic acid in water) and 75% solvent B (100% methanol) at a flow rate of 30 mL/min for about 7.62-8.34 minutes [e.g. for the fraction of (ii)].

According to specific embodiments, the conditions for HPLC comprise a 1260 Infinity preparative HPLC system coupled with an Agilent Technologies 1260 Infinity preparative HPLC system coupled with a G1364B fraction collector-1260, a G1361A 1260 prep pump, a G1365D 1260 MWD-VL detector, a C18 end caped column (e.g. Kinetex 5 μm EVO C18 100A, 250×21.2 mm column) and a mobile phase of 40% solvent A (0.1% acetic acid in water) and 60% solvent B (100% methanol) at a flow rate of 30 mL/min for about 24.24-25.07 minutes [e.g. for the fraction of (iii)].

According to specific embodiments, the conditions for HPLC comprise an 1260 Infinity preparative HPLC system coupled with an Agilent Technologies 1260 Infinity preparative HPLC system coupled with a G1364B fraction collector-1260, a G1361A 1260 prep pump, a G1365D 1260 MWD-VL detector, a C18 end caped column (e.g. Kinetex 5 μm EVO C18 100A, 250×21.2 mm column) and a mobile phase of 40% solvent A (0.1% acetic acid in water) and 60% solvent B (100% methanol) at a flow rate of 30 mL/min for about 22.88-24.23 minutes [e.g. for the fraction of (iv)].

The extracts and/or fractions obtained may be tested for cytotoxic activity.

Exemplary methods for testing cytotoxic activity are described herein below as well as in the Examples section which follows.

For testing the effect on cytotoxic activity, any in-vivo, in-vitro or ex-vivo assay known in the art for testing cytotoxic activity may be used. For example, cell viability assay on cancer cells (e.g. CTCL cells) using e.g. XTT viability assay, Alamar Blue and/or Cell sorting (e.g. for annexin V).

According to a specific embodiment, the cytotoxic activity involves cell apoptosis.

According to specific embodiments, the cytotoxic activity involves cell cycle arrest.

According to a specific embodiment, addition of a CB1 or a CB2 receptor inverse agonist (e.g. to MyLa cancer) prior to treatment with the extract, fraction and/or composition reduces the cytotoxic activity of the extract, fraction and/or composition.

According to a specific embodiment, addition of a CB2 receptor inverse agonist (e.g. to MyLa cancer cells) prior to treatment with the extract, fraction and/or composition reduces the cytotoxic activity of the extract, fraction and/or composition.

According to a specific embodiment, the extract, fraction and/or composition induces changes in expression of genes (e.g. in MyLa or Hut-78 cancer cells), as shown in Tables 12 and 13A-B hereinbelow.

According to a specific embodiment, the extract, fraction and/or composition increase IL-13 mRNA expression (e.g. in MyLa cancer cells).

The extracts and/or fractions of the present invention can also be characterized by analytical methods such as, but not limited to, spectroscopic methods such as, but not limited to, ultraviolet-visible spectroscopy (“UV-Vis”), infrared spectroscopy (“IR”), and the like; mass-spectrometry (“MS”) methods such as, but not limited to, time-of-flight MS; quadrupole MS; electrospray MS, Fourier-transform MS, Matrix-Assisted Laser Desorption/Ionization (“MALDI”), and the like; chromatographic methods such as, but not limited to, gas-chromatography (“GC”), liquid chromatograph (“LC”), high-performance liquid chromatography (“HPLC”), and the like; and combinations thereof (e.g., GC/MS, LC/MS, HPLC/UV-Vis, and the like), and other analytical methods known to persons of ordinary skill in the art.

According to an alternative or an additional embodiment, the extracts and/or fractions obtained by the methods of some embodiments of the invention are kept frozen, e.g. in a freezer, until further use (e.g. at about −20° C. to −90° C., at about −70° C. to −90° C., e.g. at −80° C.), for any required length of time.

According to an alternative or an additional embodiment, the extracts and/or fractions obtained by the methods of some embodiments of the invention are immediately used (e.g. within a few minutes e.g., up to 30 minutes).

The extracts, fractions and/or compositions obtained by the methods of some embodiments of the invention may be used separately. Alternatively, different extracts (e.g. from different plants or from separate extraction procedures) may be pooled together. Likewise, different fractions (from the same extract, from different extracts, from different plants and/or from separate extraction procedures) may be pooled together.

The term “pooled” as used herein refers to collected from the liquid chromatography (e.g. HPLC) either as a single fraction or a plurality of fractions.

According to a specific embodiment, different fractions are obtained from a single extract of Cannabis inflorescence, by subjecting the cannabis extract to liquid chromatography (e.g. HPLC) and collecting fractions comprising ingredients that are detectable by a detector operated at 220 nm (as discussed in detail herein above). According to specific embodiments these active ingredients comprise the compounds of fraction or composition (i), fraction or composition (ii), fraction or composition (iii) and/or fraction of composition (iv). Thus, for examples, fractions may be obtained at the following retention times when the following conditions are used:

HPLC comprises an Agilent Technologies 1260 Infinity preparative HPLC system coupled with a G1364B fraction collector-1260, a G1361A 1260 prep pump, a G1365D 1260 MWD-VL detector, a Kinetex 5 μm EVO C18 100A, 250×21.2 mm column and a mobile phase of 25% solvent A (0.1% acetic acid in water) and 75% solvent B (100% methanol) at a flow rate of 30 mL/min for 30 minutes followed by 15% solvent A and 85% solvent B at a flow rate of 30 mL/min for 5 minutes: D6—retention time 21.91-26.42 minutes, D2—retention time 7.62-8.34 minutes.

HPLC comprises an Agilent Technologies 1260 Infinity preparative HPLC system coupled with a G1364B fraction collector-1260, a G1361A 1260 prep pump, a G1365D 1260 MWD-VL detector, a Kinetex 5 μm EVO C18 100A, 250×21.2 mm column and a mobile phase of 40% solvent A (0.1% acetic acid in water) and 60% solvent B (100% methanol) at a flow rate of 30 mL/min for 45 minutes followed by 15% solvent A and 85% solvent B at a flow rate of 30 mL/min for 10 minutes: S5—retention time 24.24-25.07 minutes, S4—retention time 22.88-24.23 minutes.

According to an alternative or an additional embodiment, at least two, at least three or at least four of the fractions may be pooled together, at any combination thereof, as discussed in further details below.

According to specific embodiments, the fraction (e.g. liquid chromatography fraction) or the composition (as further described hereinbelow) is the fraction (e.g. liquid chromatography fraction) or the composition of (i), as defined herein.

According to specific embodiments, the fraction or the composition of (i) comprises tetrahydrocannabinol (THC).

Tetrahydrocannabinol (THC) (CAS No. 1972-08-3) as used herein encompasses native THC (i.e. originating from the Cannabis plant), or synthetic analogs or derivatives thereof. According to specific embodiments, any THC analog may be used in accordance with the present teachings as long as it comprises cytotoxic activity (alone, or as part of the composition discussed herein).

The term “analog” refers to a structural derivative having at least the same cytotoxic activity. The analog may be synthetic or naturally occurring.

According to specific embodiments, the THC comprises native THC.

Pure or synthetic THC can be commercially obtained from e.g. Restek catalog no. 34067.

The term THC does not include tetrahydrocannabinolic acid (THCA).

According to specific embodiments, the fraction or the composition of (i) comprises about 20-80% THC, about 30-70% THC, about 40-60% THC or about 45-55% THC.

According to specific embodiments, the fraction or the composition of (i) comprises 40-60% THC.

According to specific embodiments, the fraction or the composition of (i) comprises 45-55% THC.

According to specific embodiments, the fraction or the composition of (i) comprises about 50% THC.

According to specific embodiments, the fraction or the composition of (i) comprises about 51% THC.

According to specific embodiments, the fraction or the composition of (i) comprises 50-51% THC.

According to alternative or additional embodiments, the fraction or the composition of (i) comprises cannabis derived active ingredients other than the THC.

According to specific embodiments, the fraction or the composition of (i) comprises tetrahydrocannabinolic acid (THCA).

As used herein, the term “tetrahydrocannabinolic acid (THCA)” (CAS No: 23978-85-0) refers to Δ9-tetrahydrocannabinolic acid, the precursor of tetrahydrocannabinol (THC). The term THCA as used herein encompasses native THCA (i.e. originating from the Cannabis plant), or synthetic analogs or derivatives thereof. Exemplary THCA analogs include, but are not limited to, 11-0H-delta9-THCA-A and 11-Nor-delta9-THCA-A carboxylic acid [as discussed in detail in Guillermo Moreno-Sanz, Critical Review and Novel Therapeutic Perspectives of D9-Tetrahydrocannabinolic Acid A, Cannabis and Cannabinoid Research Volume 1.1, (2016)].

According to specific embodiments, the THCA comprises native THCA.

Pure or synthetic THCA can be commercially obtained from e.g. Restek catalog no. 34093.

The term THCA does not include tetrahydrocannabinol (THC).

According to specific embodiments, the fraction or the composition of (i) comprises about 20-60% THCA, about 30-60% THCA, about 35-55% THCA, about 40-60% THCA or 40-50% THCA.

According to specific embodiments, the fraction or the composition of (i) comprises less than 75%, less than 70%, less than 65%, less than 60% or less than 50% THCA.

According to specific embodiments, the fraction or the composition of (i) comprises 35-55% THCA.

According to specific embodiments, the fraction or the composition of (i) comprises 45-50% THCA.

According to specific embodiments, the fraction or the composition of (i) comprises about 47% THCA.

According to specific embodiments, the fraction or the composition of (i) comprises 46-47% THCA.

According to alternative or additional embodiments, the fraction or the composition of (i) comprises cannabis derived active ingredients other than the THCA.

According to specific embodiments, the fraction or the composition of (i) comprises THC and THCA.

According to specific embodiments, the fraction or the composition of (i) comprises 40-60% THC and 35-55% THCA.

According to specific embodiments, the fraction or the composition of (i) comprises about 50% THC and about 47% THCA.

According to specific embodiments, the fraction or the composition of (i) comprises 50-51% THC and 46-47% THCA.

According to alternative or additional embodiments, the fraction or the composition of (i) comprises cannabis derived active ingredients other than the THC and/or the THCA.

According to specific embodiments, the fraction or the composition of (i) comprises cannabinol (CBN).

Cannabinol (CBN) (CAS NO. 521-35-7) as used herein encompasses native CBN (i.e. originating from the Cannabis plant), or synthetic analogs or derivatives thereof. According to specific embodiments, any CBN analog may be used in accordance with specific embodiments of the present teachings as long as it comprises cytotoxic activity (alone, or as part of a fraction or composition discussed herein).

According to specific embodiments, the CBN comprises native CBN.

Pure or synthetic CBN can be commercially obtained from e.g. Restek catalog no. 34010.

According to specific embodiments, the fraction or the composition of (i) comprises at least about 0.1-10% CBN, at least about 0.1-5% CBN, at least about 0.1-3% CBN, at least about 0.1-2% CBN, at least about 0.5-5% CBN, at least about 0.5-3% CBN, at least about 0.5-2% CBN, at least about 1-5% CBN, at least about 1-3% CBN or at least about 1-2% CBN.

According to specific embodiments, the fraction or the composition of (i) comprises at least 1% CBN.

According to specific embodiments, the fraction or the composition of (i) comprises less than 3% CBN.

According to specific embodiments, the fraction or the composition of (i) comprises 1-2% CBN.

According to specific embodiments, the fraction or the composition of (i) comprises about 1.7% CBN.

According to specific embodiments, the fraction or the composition of (i) comprises β-caryophyllene (CAS NO. 87-44-5).

According to specific embodiments, the β-caryophyllene comprises native β-caryophyllene.

According to specific embodiments, the β-caryophyllene comprises a synthetic analog of β-caryophyllene.

According to specific embodiments, the fraction or the composition of (i) comprises at least about 0.001-1% β-caryophyllene, at least about 0.001-0.5% β-caryophyllene, at least about 0.005-0.5% β-caryophyllene, at least about 0.01-0.1% β-caryophyllene or at least about 0.01-0.05% β-caryophyllene.

According to specific embodiments, the fraction or the composition of (i) comprises about 0.01% β-caryophyllene.

According to specific embodiments, fraction or the composition of (i) comprises THC, CBN and β-caryophyllene.

According to specific embodiments, fraction or the composition of (i) comprises CBN; β-caryophyllene; and 40-60% THC, 45-55% THC, about 50% THC or 50-51% THC.

According to specific embodiments, fraction or the composition of (i) comprises CBN; β-caryophyllene; and 35-55% THCA, 40-50% THCA, about 47% THCA or 46-47% THCA.

According to specific embodiments, fraction or the composition of (i) comprises THC, THCA, CBN and β-caryophyllene.

According to specific embodiments, fraction or the composition of (i) comprises CBN; β-caryophyllene; 40-60% THC, 45-55% THC, about 50% THC or 50-51% THC; and 35-55% THCA, 40-50% THCA, about 47% THCA or 46-47% THCA.

According to specific embodiments, the fraction or the composition of (i) comprises cannabis derived active ingredients other than CBN, β-caryophyllene, THCA and THC.

According to specific embodiments, the fraction or the composition of (i) is devoid of at least one cannabinoid selected from the group consisting of cannabigerol (CBG), cannabidiol (CBD), cannabidiolic acid (CBDA), cannabigerolic acid (CBGA) and cannabichromene (CBC).

According to specific embodiments, the fraction or the composition of (i) is devoid of CBD.

According to specific embodiments, the fraction or the composition of (i) is devoid of cannabigerolic acid (CBGA) and cannabichromene (CBC).

According to specific embodiments, the fraction or the composition of (i) is devoid of cannabidiol (CBD), cannabigerolic acid (CBGA) and cannabichromene (CBC).

Cannabigerol (CBG) (CAS No. 25654-31-3) as used herein encompasses native CBG (i.e. originating from the Cannabis plant), or synthetic analogs or derivatives thereof. According to specific embodiments, any CBG analog may be used in accordance with specific embodiments of the present teachings as long as it comprises cytotoxic activity (alone, or as part of a fraction or composition discussed herein).

According to specific embodiments, the CBG comprises native CBG.

Pure or synthetic CBG can be commercially obtained from e.g. Restek catalog no. 34091.

Cannabidiol (CBD) (CAS No. 13956-29-1) as used herein encompasses native CBD (i.e. originating from the Cannabis plant), or synthetic analogs or derivatives thereof. According to specific embodiments, any CBD analog may be used in accordance with specific embodiments of the present teachings as long as it comprises cytotoxic activity (alone, or as part of a fraction or composition discussed herein).

Exemplary CBD analogs include, but are not limited to, (−)-DMH-CBD-11-oic acid, HU-308 (commercially available e.g. from Tocris Bioscience, 3088), 0-1602 (commercially available e.g. from Tocris Bioscience 2797/10), DMH-CBD (commercially available e.g. from Tocris Bioscience, 1481) [as discussed in detail in Burstein S, Bioorg Med Chem. (2015) 23(7): 1377-85], Abn-CBD, HUF-101. CBDV, CBDM, CBND-05, CBND-C3, 6-Hydroxy-CBD-triacetate or CBD-aldehyde-diacetate [as discussed in detail in An Overview on Medicinal Chemistry of Synthetic and Natural Derivatives of Cannabidiol, Frontiers in Pharmacology, June 2017 I Volume 8 I Article 422].

According to specific embodiments, the CBD comprises native CBD.

Pure or synthetic CBD can be commercially obtained from e.g. Restek catalog no. 34011.

Cannabidiolic acid (CBDA) (CAS No. 1244-58-2) as used herein encompasses native CBD1 (i.e. originating from the Cannabis plant), or synthetic analogs or derivatives thereof.

According to specific embodiments, the CBDA comprises native CBDA.

Pure or synthetic CBDA can be commercially obtained from e.g. Restek catalog no. 34099.

Cannabigerolic acid (CBGA) (CAS No. 25555-57-1) is the precursor of THCA, CBDA, and CBCA. The term CBGA as used herein encompasses native CBGA (i.e. originating from the Cannabis plant) or synthetic analogs or derivatives thereof. An exemplary CBGA analog includes, but is not limited to, CBGVA.

According to specific embodiments, the CBGA comprises native CBGA.

Pure or synthetic CBGA can be commercially obtained from e.g. Sigma-Aldrich catalog no. C-142.

Cannabichromene (CBC) (CAS NO. 20675-51-8) as used herein encompasses native CBC (i.e. originating from the Cannabis plant), or synthetic analogs or derivatives thereof. According to specific embodiments, any CBC analog may be used in accordance with specific embodiments of the present teachings as long as it comprises cytotoxic activity (alone, or as part of a fraction or composition discussed herein).

According to specific embodiments, the CBC comprises native CBC.

Pure or synthetic CBC can be commercially obtained from e.g. Restek catalog no. 34092.

According to specific embodiments, the fraction or the composition of (i) is devoid of cannabis derived active ingredients other than THC, THCA, CBN and β-caryophyllene.

According to specific embodiments, the fraction or the composition of (i) is devoid of cannabis derived active ingredients other than THC, THCA and CBN.

According to specific embodiments, the fraction or the composition of (i) comprises components as listed in Table 9, hereinbelow.

According to specific embodiments, the fraction or the composition of (i) comprises at least one, two, three or four components as listed in Table 9 hereinbelow.

According to specific embodiments, the fraction or the composition of (i) comprises THC, THCA, CBN and/or β-caryophyllene in a concentration as listed in Table 9±10%.

According to specific embodiments, the fraction or the composition of (i) comprises THC, THCA, CBN and β-caryophyllene in a concentration as listed in Table 9±10%.

According to specific embodiments, the fraction or the composition of (i) comprises THC, THCA and CBN in a concentration as listed in Table 9±10%.

According to specific embodiments, the fraction or the composition of (i) comprises THC, THCA, CBN and β-caryophyllene in a concentration as listed in Table 9.

According to specific embodiments, the fraction or the composition of (i) comprises THC, THCA and CBN in a concentration as listed in Table 9.

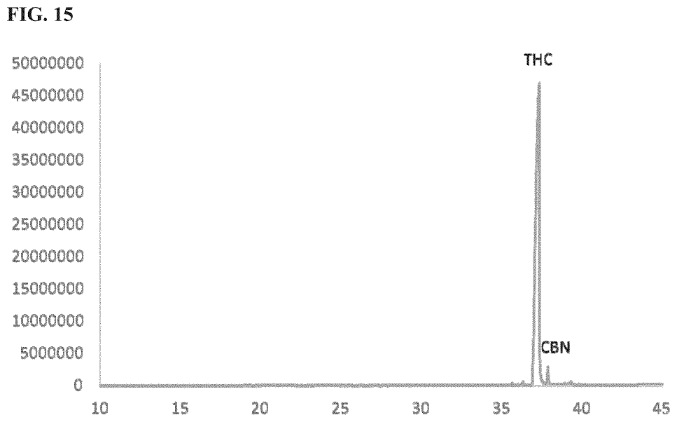

According to specific embodiments, the fraction of (i) comprises fraction D6 as shown in Table 9 hereinbelow and FIG. 15.

According to specific embodiments, the fraction (e.g. liquid chromatography fraction) or the composition (as further described hereinbelow) is the fraction (e.g. liquid chromatography fraction) or the composition of (ii), as defined herein.

According to specific embodiments, the fraction or the composition of (ii) comprises at least two cannabinoids selected from the group consisting of cannabidiol (CBD), cannabichromene (CBC) and cannabigerol (CBG), wherein a concentration of the CBD, the CBC and/or the CBG in the composition is 5-30% CBD, 0.1-10% CBC and/or 0.1-15% CBG.

According to specific embodiments, the fraction or the composition of (ii) comprises at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde.

According to specific embodiments, the fraction or the composition of (ii) comprises at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde.

According to specific embodiments, the fraction or the composition of (ii) comprises one, two or all of the CBD, CBC and CBG.

According to specific embodiments, the fraction or the composition of (ii) comprises CBD.

According to specific embodiments, the fraction or the composition of (ii) comprises at least about 5-30% CBD, at least about 5-20% CBD or at least about 10-20% CBD.

According to specific embodiments, the fraction or the composition of (ii) comprises at least 5% CBD, at least 10% CBD or at least 15% CBD.

According to specific embodiments, the fraction or the composition of (ii) comprises less than 70% CBD, less than 60% CBD, less than 50% CBD, less than 40% CBD, less than 30% CBD, or less than 20% CBD.

According to specific embodiments, the fraction or the composition of (ii) comprises 15-20% CBD.

According to specific embodiments, the fraction or the composition of (ii) comprises 15-17% CBD.

According to specific embodiments, the fraction or the composition of (ii) comprises about 16% CBD.

According to specific embodiments, the fraction or the composition of (ii) comprises about 16.5% CBD.

According to specific embodiments, the fraction or the composition of (ii) comprises CBC.

According to specific embodiments, the fraction or the composition of (ii) comprises at least about 0.1-10% CBC, at least about 0.1-5% CBC, at least about 0.5-10% CBC, at least about 0.5-5% CBC or at least about 1-5% CBC.

According to specific embodiments, the fraction or the composition of (ii) comprises at least 1% CBC, at least 2% CBC or at least 3% CBC.

According to specific embodiments, the fraction or the composition of (ii) comprises less than 70% CBC, less than 60% CBC, less than 50% CBC, less than 40% CBC, less than 30% CBC, less than 20% CBC, less than 10% CBC or less than 5% CBC.

According to specific embodiments, the fraction or the composition of (ii) comprises 2-4% CBC.

According to specific embodiments, the fraction or the composition of (ii) comprises about 3% CBC.

According to specific embodiments, the fraction or the composition of (ii) comprises CBG.

According to specific embodiments, the fraction or the composition of (ii) comprises at least about 0.1-15% CBG, at least about 0.1-10% CBG or at least about 1-9% CBG.

According to specific embodiments, the fraction or the composition of (ii) comprises at least 1% CBG, at least 2% CBG, at least 3% CBG or at least 5% CBG.

According to specific embodiments, the fraction or the composition of (ii) comprises less than 70% CBG, less than 60% CBG, less than 50% CBG, less than 40% CBG, less than 30% CBG, less than 20% CBG or less than 10% CBG.

According to specific embodiments, the fraction or the composition of (ii) comprises less than 9% CBG.

According to specific embodiments, the fraction or the composition of (ii) comprises 5-9% CBG.

According to specific embodiments, the fraction or the composition of (ii) comprises about 6% CBG.

According to specific embodiments, the fraction or the composition of (ii) comprises about 6.5% CBG.

According to specific embodiments the fraction or the composition of (ii) comprises CBC and CBD.

According to specific embodiments the fraction or the composition of (ii) comprises CBC and CBG.

According to specific embodiments the fraction or the composition of (ii) comprises CBD and CBG.

According to specific embodiments the fraction or the composition of (ii) comprises CBC, CBD and CBG.

According to specific embodiments, the fraction or the composition of (ii) comprises at least one, at least two, at least three, at least 4, at least 5, at least 6, at least 7, at least 8, at least 9 or at least 10 of c-Eudesmol, Longifolene, Agarospirol, Neoisolongifolene, Ledol, Germacrene D, c-Maaliene, bisabolene, caryophyllene oxide and Longifolenaldehyde.

According to specific embodiments, any of c-Eudesmol, Longifolene, Agarospirol, Neoisolongifolene, Ledol, Germacrene D, c-Maaliene, bisabolene, caryophyllene oxide and Longifolenaldehyde comprises a native cannabinoid.

According to specific embodiments, the c-Eudesmol, Longifolene, Agarospirol, Neoisolongifolene, Ledol, Germacrene D, c-Maaliene, bisabolene, caryophyllene oxide and Longifolenaldehyde, comprises a synthetic analog thereof.

According to specific embodiments, the fraction or the composition of (ii) comprises c-Eudesmol (CAS NO: 1209-71-8).

According to specific embodiments, the fraction or the composition of (ii) comprises at least about 0.2-1% c-Eudesmol.

According to specific embodiments, the fraction or the composition of (ii) comprises 0.6-0.7% c-Eudesmol.

According to specific embodiments, the fraction or the composition of (ii) comprises about 0.68% c-Eudesmol.

According to specific embodiments, the fraction or the composition of (ii) comprises Longifolene (CAS NO: 475-20-7).

According to specific embodiments, the fraction or the composition of (ii) comprises at least about 1-5% Longifolene.

According to specific embodiments, the fraction or the composition of (ii) comprises 2-3% Longifolene.

According to specific embodiments, the fraction or the composition of (ii) comprises about 2.6% Longifolene.

According to specific embodiments, the fraction or the composition of (ii) comprises Agarospirol (CAS NO: 1460-73-7).

According to specific embodiments, the fraction or the composition of (ii) comprises at least about 1-5% Agarospirol.

According to specific embodiments, the fraction or the composition of (ii) comprises 2-3% Agarospirol.

According to specific embodiments, the fraction or the composition of (ii) comprises about 2.3% Agarospirol.

According to specific embodiments, the fraction or the composition of (ii) comprises Neoisolongifolene.

According to specific embodiments, the fraction or the composition of (ii) comprises at least about 1-5% Neoisolongifolene.

According to specific embodiments, the fraction or the composition of (ii) comprises 2-3% Neoisolongifolene.

According to specific embodiments, the fraction or the composition of (ii) comprises about 2.8% Neoisolongifolene.

According to specific embodiments, the fraction or the composition of (ii) comprises c Ledol (CAS NO: 577-27-5).

According to specific embodiments, the fraction or the composition of (ii) comprises at least about 0.5-1% Ledol.

According to specific embodiments, the fraction or the composition of (ii) comprises 0.7-0.8% Ledol.

According to specific embodiments, the fraction or the composition of (ii) comprises about 0.73% Ledol.

According to specific embodiments, the fraction or the composition of (ii) comprises Germacrene D (CAS NO: 28387-44-2).

According to specific embodiments, the fraction or the composition of (ii) comprises at least about 1-5% Germacrene D.

According to specific embodiments, the fraction or the composition of (ii) comprises 2-4% Germacrene D.

According to specific embodiments, the fraction or the composition of (ii) comprises about 3% Germacrene D.

According to specific embodiments, the fraction or the composition of (ii) comprises c-Maaliene.

According to specific embodiments, the fraction or the composition of (ii) comprises at least about 0.1-1% c-Maaliene.

According to specific embodiments, the fraction or the composition of (ii) comprises 0.5-0.8% c-Maaliene.

According to specific embodiments, the fraction or the composition of (ii) comprises about 0.68% c-Maaliene.

According to specific embodiments, the fraction or the composition of (ii) comprises c-bisabolene.

According to specific embodiments, the fraction or the composition of (ii) comprises at least about 0.5-5% bisabolene.

According to specific embodiments, the fraction or the composition of (ii) comprises 0.5-1.5% bisabolene.

According to specific embodiments, the fraction or the composition of (ii) comprises about 1.2% bisabolene.

According to specific embodiments, the fraction or the composition of (ii) comprises caryophyllene oxide (CAS NO: 1139-30-6).

According to specific embodiments, the fraction or the composition of (ii) comprises at least about 0.1-1% caryophyllene oxide.

According to specific embodiments, the fraction or the composition of (ii) comprises 0.1-0.5% caryophyllene oxide.

According to specific embodiments, the fraction or the composition of (ii) comprises about 0.3% caryophyllene oxide.

According to specific embodiments, the fraction or the composition of (ii) comprises c-Longifolenaldehyde (CAS NO: 79645-28-6).

According to specific embodiments, the fraction or the composition of (ii) comprises at least about 0.1-1% Longifolenaldehyde.

According to specific embodiments, the fraction or the composition of (ii) comprises 0.4-0.5% Longifolenaldehyde.

According to specific embodiments, the fraction or the composition of (ii) comprises about 0.45% Longifolenaldehyde.

According to specific embodiments, the fraction or the composition of (ii) comprises all of said c-Eudesmol, said Longifolene, said Agarospirol, said Neoisolongifolene, said Ledol, said Germacrene D, said c-Maaliene, said bisabolene, said caryophyllene oxide and said Longifolenaldehyde.

According to specific embodiments, the fraction or the composition of (ii) is devoid of tetrahydrocannabinolic acid (THCA).

According to specific embodiments, the fraction or the composition of (ii) is devoid of cannabigerolic acid (CBGA) and/or cannabidiolic acid (CBDA).

According to specific embodiments, the fraction or the composition of (ii) is devoid of cannabinol (CBN).

According to specific embodiments, the fraction or the composition of (ii) comprises is devoid of cannabis derived active ingredients other than CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde.

According to specific embodiments, the fraction or the composition of (ii) comprises is devoid of cannabis derived active ingredients other than CBC, CBD and CBG.

According to specific embodiments, the fraction or the composition of (ii) comprises components as listed in Table 8, hereinbelow.

According to specific embodiments, the fraction or the composition of (ii) comprises at least one, at least two, at least three, at least four, at least five, at least six, at least seven, at least eight, at least 9 or at least 10 components as listed in Table 8 hereinbelow.

According to specific embodiments, the fraction or the composition of (ii) comprises CBC, CBD, CBG c-Eudesmol, Longifolene, Agarospirol, Neoisolongifolene, Ledol, Germacrene D, c-Maaliene, bisabolene, d caryophyllene oxide and/or Longifolenaldehyde, in a concentration as listed in Table 8±10%.

According to specific embodiments, the fraction or the composition of (ii) comprises CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde in a concentration as listed in Table 8±10%.

According to specific embodiments, the fraction or the composition of (ii) comprises CBC, CBD, CBG, c-Eudesmol, Longifolene, Agarospirol, Neoisolongifolene, Ledol, Germacrene D, c-Maaliene, bisabolene, caryophyllene oxide and/or Longifolenaldehyde, in a concentration as listed in Table 8.

According to specific embodiments, the fraction or the composition of (ii) comprises CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde in a concentration as listed in Table 8.

According to specific embodiments, the fraction or the composition of (ii) comprises CBC, CBD and/or CBG, in a concentration as listed in Table 8±10%.

According to specific embodiments, the fraction or the composition of (ii) comprises CBC, CBD and/or CBG, in a concentration as listed in Table 8.

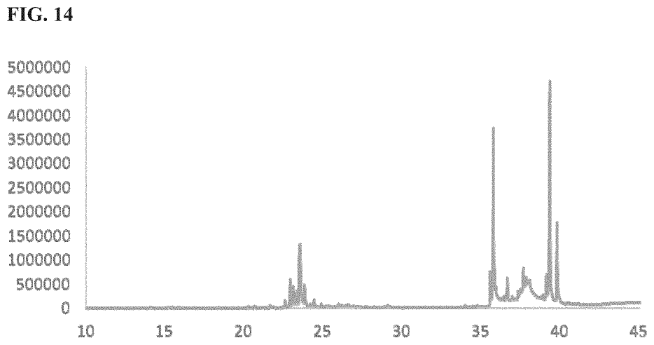

According to specific embodiments, the fraction of (ii) comprises fraction D2 as shown in Table 8 hereinbelow and FIG. 14.

According to specific embodiments, the fraction (e.g. liquid chromatography fraction) or the composition (as further described hereinbelow) is the fraction (e.g. liquid chromatography fraction) or the composition of (iii), as defined herein.

According to specific embodiments, the fraction or the composition of (iii) comprises at least one cannabinoid selected from the group consisting of cannabidiol (CBD), cannabichromene (CBC), tetrahydrocannabinol (THC) and cannabigerol (CBG).

According to specific embodiments, the fraction or the composition of (iii) comprises at least one cannabinoid selected from the group consisting of cannabidiol (CBD), cannabichromene (CBC), tetrahydrocannabinol (THC) and at least 50% cannabigerol (CBG).

According to specific embodiments, the fraction or the composition of (iii) comprises at least one cannabinoid selected from the group consisting of cannabidiol (CBD), cannabichromene (CBC), tetrahydrocannabinol (THC) and cannabigerol (CBG), and devoid of tetrahydrocannabinolic acid (THCA).

According to specific embodiments, the fraction or the composition of (iii) comprises at least 50% cannabigerol (CBG) and a cannabinoid selected from the group consisting of cannabidiol (CBD), cannabichromene (CBC) and tetrahydrocannabinol (THC).

According to specific embodiments, the fraction or the composition of (iii) comprises one, two, three or all of the CBD, CBC, THC and CBG.

According to specific embodiments, the fraction or the composition of (iii) comprises CBD.

According to specific embodiments, the fraction or the composition of (iii) comprises at least about 20-50% CBD, at least about 25-45% CBD or at least about 30-40% CBD.

According to specific embodiments, the fraction or the composition of (iii) comprises at least 10% CBD, at least 20% CBD or at least 30% CBD.

According to specific embodiments, the fraction or the composition of (iii) comprises at least 30% CBD.

According to specific embodiments, the fraction or the composition of (ii) comprises less than 70% CBD, less than 60% CBD, less than 50% CBD, or less than 40% CBD.

According to specific embodiments, the fraction or the composition of (iii) comprises 30-40% CBD.

According to specific embodiments, the fraction or the composition of (iii) comprises 35-50% CBD.

According to specific embodiments, the fraction or the composition of (iii) comprises about 38% CBD.

According to specific embodiments, the fraction or the composition of (iii) comprises CBC.

According to specific embodiments, the fraction or the composition of (iii) comprises at least about 0.1-10% CBC, at least about 0.1-5% CBC, at least about 0.1-1% CBC or at least about 0.1-0.5% CBC.

According to specific embodiments, the fraction or the composition of (iii) comprises less than 70% CBC, less than 60% CBC, less than 50% CBC, less than 40% CBC, less than 30% CBC, less than 20% CBC, less than 10% CBC, less than 5% CBC, less than 1% CBC or less than 0.5% CBC.

According to specific embodiments, the fraction or the composition of (iii) comprises less than 2% CBC.

According to specific embodiments, the fraction or the composition of (iii) comprises 0.4-0.5% CBC.

According to specific embodiments, the fraction or the composition of (iii) comprises about 0.44% CBC.

According to specific embodiments, the fraction or the composition of (iii) comprises THC.

According to specific embodiments, the fraction or the composition of (iii) comprises at least about 0.1-10% THC, at least about 0.1-5% THC or at least about 0.1-1% THC.

According to specific embodiments, the fraction or the composition of (iii) comprises less than 70% THC, less than 60% THC, less than 50% THC, less than 40% THC, less than 30% THC, less than 20% THC, less than 10% THC, less than 5% THC or less than 1% THC.

According to specific embodiments, the fraction or the composition of (iii) comprises less than 2% THC.

According to specific embodiments, the fraction or the composition of (iii) comprises 0.6-0.8% THC.

According to specific embodiments, the fraction or the composition of (iii) comprises about 0.7% THC.

According to specific embodiments, the fraction or the composition of (iii) comprises CBG.

According to specific embodiments, the fraction or the composition of (iii) comprises at least about 30-70% CBG, at least about 40-70% CBG or at least about 50-70% CBG.

According to specific embodiments, the fraction or the composition of (iii) comprises at least 40% CBG or at least 50% CBG.

According to specific embodiments, the fraction or the composition of (iii) comprises at least 50% CBG.

According to specific embodiments, the fraction or the composition of (iii) comprises less than 90% CBG, less than 80% CBG, less than 70% CBG or less than 60% CBG.

According to specific embodiments, the fraction or the composition of (iii) comprises 50-65% CBG.

According to specific embodiments, the fraction or the composition of (iii) comprises about 59% CBG.

According to specific embodiments the fraction or the composition of (iii) comprises at least two of CBD, CBC, THC and CBG.

According to specific embodiments the fraction or the composition of (iii) comprises CBD and CBC, CBD and THC, CBD and CBG, CBC and THC, CBC and CBG, or THC and CBG.

According to specific embodiments the fraction or the composition of (ii) comprises CBD, CBC and THC; CBD, CBC and CBG; or CBC, THC and CBG.

According to specific embodiments the fraction or the composition of (iii) comprises all of CBD, CBC, THC and CBG.

According to specific embodiments the fraction or the composition of (iii) comprises at least 30% CBD, less than 2% CBC, less than 2% THC and at least 50% CBG.

According to specific embodiments the fraction or the composition of (iii) comprises about 38.25% CBD, about 0.44% CBC, about 0.74% THC and/or about 58.85% CBG.

According to specific embodiments the fraction or the composition of (iii) comprises about 38.25% CBD, about 0.44% CBC, about 0.74% THC and about 58.85% CBG.

According to specific embodiments the fraction or the composition of (iii) is devoid of tetrahydrocannabinolic acid (THCA).

According to specific embodiments the fraction or the composition of (iii) is devoid of cannabigerolic acid (CBGA) and/or cannabidiolic acid (CBDA).

According to specific embodiments, the fraction or the composition of (iii) is devoid of cannabinol (CBN).

According to specific embodiments the fraction or the composition of (iii) is devoid of cannabis derived active ingredients other than CBD, CBC, THC and CBG.

According to specific embodiments, the fraction or the composition of (iii) comprises components as listed in Table 4, hereinbelow.

According to specific embodiments, the fraction or the composition of (iii) comprises at least one, two, three or four components as listed in Table 4 hereinbelow.

According to specific embodiments, the fraction or the composition of (iii) comprises CBD, CBC, THC and/or CBG in a concentration as listed in Table 4±10%.

According to specific embodiments, the fraction or the composition of (iii) comprises CBD, CBC, THC and CBG in a concentration as listed in Table 4±10%.

According to specific embodiments, the fraction or the composition of (iii) comprises CBD, CBC, THC and/or CBG in a concentration as listed in Table 4.

According to specific embodiments, the fraction or the composition of (iii) comprises CBD, CBC, THC and CBG in a concentration as listed in Table 4.

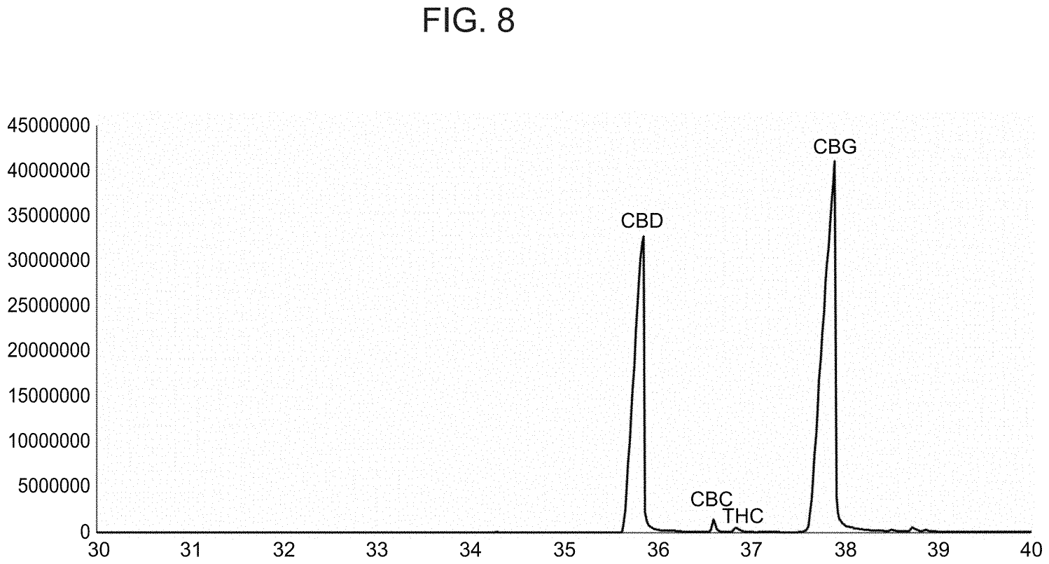

According to specific embodiments, the fraction of (iii) comprises fraction S5 as shown in Table 4 hereinbelow and FIG. 8.

According to specific embodiments, the fraction (e.g. liquid chromatography fraction) or the composition (as further described hereinbelow) is the fraction (e.g. liquid chromatography fraction) or the composition of (iv), as defined herein.

According to specific embodiments, the fraction or the composition of (iv) comprises cannabidiol (CBD).

According to specific embodiments, the fraction or the composition of (iv) comprises at least 90% CBD, at least 91% CBD, at least 92% CBD, at least 93% CBD, at least 94% CBD, at least 95% CBD, at least 96% CBD, at least 97% CBD, at least 98% CBD, at least 99% CBD or 100% CBD.

According to specific embodiments, the fraction or the composition of (iv) comprises at least 90% CBD.

According to specific embodiments, the fraction of the composition of (iv) comprises 80-99% CBD, 85-99% CBD, 90-99% CBD or 95-99% CBD.

According to specific embodiments, the fraction or the composition of (iv) comprises at least one cannabinoid other than CBD.

According to specific embodiments, the fraction or the composition of (iv) comprises THC, CBG, CBDV and/or a-bisabolol.

According to specific embodiments, the fraction or the composition of (iv) comprises one, two, three or all of the THC, CBG, CBDV and a-bisabolol.

According to specific embodiments, the fraction or the composition of (iv) comprises THC.

According to specific embodiments, the fraction or the composition of (iv) comprises less than 2% THC, less than 1% THC or less than 0.5% THC.

According to specific embodiments, the fraction or the composition of (iv) comprises about 0.3% THC.

According to specific embodiments, the fraction or the composition of (iv) comprises CBG.

According to specific embodiments, the fraction or the composition of (iv) comprises less than 2% CBG, less than 1% CBG, less than 0.5% CBG or less than 0.3% CBG.

According to specific embodiments, (iv) comprises about 0.2% CBG.

According to specific embodiments, the fraction or the composition of (iv) comprises CBDV.

According to specific embodiments, the fraction or the composition of (iv) comprises less than 2% CBDV, less than 1% CBDV, less than 0.5% CBDV or less than 0.2% CBDV.

According to specific embodiments, (iv) comprises about 0.1% CBDV.

According to specific embodiments, the fraction or the composition of (iv) comprises a-bisabolol (CAS NO. 23089-26-1).

According to specific embodiments, the fraction or the composition of (iv) comprises less than 3% CBDV, less than 2% CBDV or less than 1% a-bisabolol.

According to specific embodiments, (iv) comprises about 0.9% a-bisabolol.

According to specific embodiments, the fraction or the composition of (iv) is devoid of a cannabinoid selected from the group consisting of cannabinol (CBN), cannabichromene (CBC), cannabigerol (CBG), tetrahydrocannabinol (THC), cannabigerolic acid (CBGA) and cannabidiolic acid (CBDA).

According to specific embodiments, the fraction or the composition of (iv) is devoid of a cannabinoid selected from the group consisting of cannabinol (CBN), cannabichromene (CBC), cannabigerolic acid (CBGA) and cannabidiolic acid (CBDA).

According to specific embodiments, the fraction or the composition of (iv) is devoid of tetrahydrocannabinolic acid (THCA).

According to specific embodiments, the fraction or the composition of (iv) is devoid of cannabinol (CBN).

According to specific embodiments, the fraction or the composition of (iv) is devoid of a cannabinoid selected from the group consisting of cannabichromene (CBC), cannabigerol (CBG) and tetrahydrocannabinol (THC).

According to specific embodiments, the fraction or the composition of (iv) is devoid of cannabis derived active ingredients other than CBD.

According to specific embodiments, the fraction or the composition of (iv) is devoid of cannabis derived active ingredients other than CBD, THC, CBG, CBDV and a-bisabolol.

According to specific embodiments, the fraction of (iv) comprises fraction S4 as shown in Table 3 hereinbelow and FIG. 7.

According to specific embodiments, a concentration of CBD, CBC, THC and/or CBG in the fraction or the composition of (iii) or (iv) is 50-90% CBD, 10-40% CBG, 0.15-0.5 THC and/or 0.09-0.3% CBC.

According to specific embodiments, the fraction or the composition of (iii) or (iv) comprises 50-90% CBD, 10-40% CBG, 0.15-0.5 THC and 0.09-0.3% CBC.

According to specific embodiments, a concentration of CBD, CBC, THC and/or CBG in said liquid chromatography fraction or said composition of (iii) or (iv) is as listed in Table 5.

According to specific embodiments, the fraction or composition of (iii) or (iv) is as listed in Table 5.

Specific embodiments of the present invention are also directed to a product prepared by the process disclosed herein.

Hence, according to an aspect of the present invention, there is provided a cytotoxic composition obtainable by the methods disclosed herein.

Thus, according to specific embodiments, the composition disclosed herein comprises a liquid chromatography fraction of cannabis extract.

According to an alternative or an additional aspect of the present invention, there is provided a composition comprising a liquid chromatography fraction of a cannabis extract selected from the group consisting of:

(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and/or cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and/or cannabigerol (CBG), wherein said liquid chromatography fraction comprises at least 50% CBG and/or wherein said liquid chromatography fraction is devoid of tetrahydrocannabinolic acid (THCA) and wherein said liquid chromatography fraction comprises said CBD said liquid chromatography fraction comprises at least two of said cannabinoids; and

(iv) a liquid chromatography fraction comprising cannabidiol (CBD), wherein said CBD is at least 90% CBD and/or wherein said liquid chromatography fraction is devoid of tetrahydrocannabinolic acid (THCA).

According to an alternative or an additional aspect of the present invention, there is provided a composition comprising a liquid chromatography fraction of a cannabis extract selected from the group consisting of:

(i) a liquid chromatography fraction comprising 40-60% tetrahydrocannabinol (THC);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fraction is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and/or cannabigerol (CBG), wherein said liquid chromatography fraction comprises at least 50% CBG and/or wherein said liquid chromatography fraction is devoid of tetrahydrocannabinolic acid (THCA) and wherein said liquid chromatography fraction comprises said CBD said liquid chromatography fraction comprises at least two of said cannabinoids; and

(iv) a liquid chromatography fraction comprising at least 90% cannabidiol (CBD) and at least one cannabinoid other than said CBD.

The present invention, in some embodiments thereof, also contemplates synthetic compositions comprising synthetic cannabinoids and compositions comprising purified cannabinoids, based on the cannabinoid composition of the cytotoxic compositions obtainable by the methods disclosed herein.

According to specific embodiments, the composition disclosed herein is a synthetic composition.

According to specific embodiments, the cannabinoids in the composition disclosed herein are purified from cannabis.

Thus, according to an alternative or an additional aspect of the present invention, there is provided a composition selected from the group consisting of:

(i) a composition comprising 40-60% tetrahydrocannabinol (THC) and at least one cannabinoid selected form the group consisting of tetrahydrocannabinolic acid (THCA), cannabinol (CBN) and β-caryophyllene;

(ii) a composition comprising at least two cannabinoids selected from the group consisting of cannabidiol (CBD), cannabichromene (CBC) and cannabigerol (CBG), wherein a concentration of said CBD, said CBC and/or said CBG in said composition is 5-30% CBD, 0.1-10% CBC and/or 0.1-15% CBG; and

(iii) a composition comprising at least 50% cannabigerol (CBG) and at least one cannabinoid selected from the group consisting of cannabidiol (CBD), cannabichromene (CBC) and tetrahydrocannabinol (THC).

According to an alternative or an additional aspect of the present invention, there is provided an article of manufacture comprising at least one of compositions (i)-(iii) and a composition (iv) comprising at least 90% CBD and at least one cannabinoid other than said CBD.

According to an alternative or an additional aspect of the present invention, there is provided a composition selected from the group consisting of:

(i) a composition comprising 40-60% tetrahydrocannabinol (THC) and at least one cannabinoid selected form the group consisting of tetrahydrocannabinolic acid (THCA), cannabinol (CBN) and β-caryophyllene;

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; and

(iii) a composition comprising at least 50% cannabigerol (CBG) and at least one cannabinoid selected from the group consisting of cannabidiol (CBD), cannabichromene (CBC) and tetrahydrocannabinol (THC).

According to an alternative or an additional aspect of the present invention, there is provided an article of manufacture comprising at least one of compositions (i)-(iii) and a composition (iv) comprising CBD.

According to specific embodiments, the article of manufacture comprise composition (iii) and composition (iv).

According to specific embodiments the at least one of compositions (i)-(iii) and the composition (iv) are packaged in separate formulations.

According to specific embodiments the at least one of compositions (i)-(iii) and the composition (iv) are in a co-formulation.

As mentioned hereinabove, according to specific embodiments, at least two, at least three or at least four of the fractions or compositions may be pooled together, at any combination thereof.

According to specific embodiments, the at least two, at least three or at least 4 of the fractions or compositions pooled together are provided in separate formulation.

According to specific embodiments, the at least two, at least three or at least 4 of the fractions or compositions pooled together are provided in a co-formulation.

According to specific embodiments, the at least two, at least three or at least 4 of the fractions or compositions pooled together have a combined synergistic cytotoxic activity on cutaneous T cell lymphoma cells (CTCL) as compared to each of the fractions or compositions.

According to a specific embodiment, the at least two, at least three or at least 4 of the fractions or composition pooled together have a combined synergistic effect on IL-13 mRNA expression (e.g. in MyLa cancer cells).

Thus, according to specific embodiments, the fraction, the composition or the article of manufacture disclosed herein comprises at least two of the fractions or the compositions disclosed herein.

According to specific embodiments, the concentration:concentration w/v:w/v (μg/ml to μg/ml) ratio between the fraction or composition of (iii) and the fraction or composition (iv) in the at least two fractions or compositions is 0.01:1-100:1, 0.1:1-10:1, 0.5:1-5:1, 0.5:1-2:1, or 0.25:1-0.5:1.

According to specific embodiments, the total concentration of CBD, CBC, THC and/or CBG in the at least two of the fractions or the compositions is 50-90% CBD, 10-40% CBG, 0.15-0.5 THC and/or 0.09-0.3% CBC.

According to specific embodiments, the total concentration of CBD, CBC, THC and/or CBG in the at least two of said liquid chromatography fractions or said compositions is as listed in Table 5±10%.

According to specific embodiments, the total concentration of CBD, CBC, THC and CBG in the at least two of said liquid chromatography fractions or said compositions is as listed in Table 5±10%.

According to specific embodiments, the total concentration of CBD, CBC, THC and/or CBG in the at least two of said liquid chromatography fractions or said compositions is as listed in Table 5.

According to specific embodiments, the total concentration of CBD, CBC, THC and CBG in the at least two of said liquid chromatography fractions or said compositions is as listed in Table 5.

According to specific embodiments, a molar ratio of CBC to CBD, a molar ratio of THC to CBD and/or a molar ratio of CBG to CBD in the at least two of said liquid chromatography fractions or said compositions is as listed in Table 5±10%.

According to specific embodiments, a molar ratio of CBC to CBD, a molar ratio of THC to CBD and/or a molar ratio of CBG to CBD in the at least two of said liquid chromatography fractions or said compositions is as listed in Table 5.

As the present invention have generated a novel composition comprising fractions S5 and S4, according to an alternative or an additional aspect of the present invention, there is provided a composition comprising 50-90% cannabidiol (CBD), 10-40% cannabigerol (CBG) and 0.15-0.5% tetrahydrocannabinol (THC), referred to hereinafter as composition (v). According to specific embodiments, composition (v) further comprises 0.09-0.3% cannabichromene (CBC).

Thus, according to specific embodiments, composition (v) comprises 50-90% CBD, 10-40% CBG, 0.15-0.5 THC and 0.09-0.3% CBC.

According to specific embodiments, composition (v) comprises CBD, CBG, THC and/or CBC in a concentration as listed in Table 5±10%.

According to specific embodiments, composition (v) comprises CBD, CBG and THC in a concentration as listed in Table 5±10%.

According to specific embodiments, composition (v) comprises CBD, CBG, THC and/or CBC in a concentration as listed in Table 5.

According to specific embodiments, composition (v) comprises CBD, CBG and THC in a concentration as listed in Table 5.

An additional or an alternative way of describing the contents of composition (v) uses a molar ratio between the cannabinoids in the composition.

Hence, according to an alternative or an additional aspect of the present invention, there is provided a composition comprising cannabidiol (CBD), cannabigerol (CBG) and tetrahydrocannabinol (THC) in molar ratios as listed in Table 5, this composition is also referred to hereinafter as composition (v).

According to specific embodiments, composition (v) further comprises cannabichromene (CBC) in a molar ratio as listed in Table 5.

According to specific embodiments, composition (v) is devoid of THCA.

According to specific embodiments, composition (v) is devoid of CBGA and/or CBDA.

According to specific embodiments, composition (iv) is devoid of cannabinoids other than CBD, CBG, THC and CBC.

According to specific embodiments, composition (v) is as listed in Table 5.

According to specific embodiments, composition (v) comprises a combination of fractions S5 and S4 as shown in Tables 3-5 hereinbelow.

According to specific embodiments, the at least two fractions or compositions comprise the fraction or composition of (i) and the fraction or composition (ii).

According to specific embodiments, the concentration to concentration w/v:w/v (μg/ml to μg/ml) ratio between the fraction or composition of (i) and the fraction or composition (ii) in the at least two fractions or compositions is 0.01:1-100:1, 0.1:1-10:1, 0.5:1-5:1, 1.25:1-5:1, 0.4:1-1:1 or about 0.4:1.

According to specific embodiments, the total concentration of THC, THCA, CBN, β-caryophyllene, CBC, CBD and/or CBG, in the at least two of said compositions is 14-43% THC, 13-40% THCA, 0.5-1.5% CBN, 0-0.05 β-caryophyllene, 0.5-2.2% CBC, 2.5-12% CBD and/or 1-5% CBG.

According to specific embodiments, the total concentration of THC, THCA, CBN, CBC, CBD and/or CBG, in the at least two of said compositions is 14-43% THC, 13-40% THCA, 0.5-1.5% CBN, 0.5-2.2% CBC, 2.5-12% CBD and/or 1-5% CBG.

According to specific embodiments, a total concentration of THC, THCA, CBN, β-caryophyllene, CBC, CBD and/or CBG, in the at least two of said liquid chromatography fractions or said compositions is as listed in Table 10±10%.

According to specific embodiments, a total concentration of THC, THCA, CBN, CBC, CBD and/or CBG, in the at least two of said liquid chromatography fractions or said compositions is as listed in Table 10±10%.

According to specific embodiments, a total concentration of THC, THCA, CBN, β-caryophyllene, CBC, CBD and/or CBG, in the at least two of said liquid chromatography fractions or said compositions is as listed in Table 10.

According to specific embodiments, a total concentration of THC, THCA, CBN, CBC, CBD and/or CBG, in the at least two of said liquid chromatography fractions or said compositions is as listed in Table 10.

According to specific embodiments, the total concentration of THC, THCA, CBN, β-caryophyllene, CBC, CBD, CBG, c-Eudesmol, Longifolene, Agarospirol, Neoisolongifolene, Ledol, Germacrene D, c-Maaliene, bisabolene, caryophyllene oxide and/or Longifolenaldehyde, in the at least two of said liquid chromatography fractions or the compositions is 14-43% THC, 13-40% THCA, 0.5-1.5% CBN, 0-0.05 β-caryophyllene, 0.5-2.2% CBC, 2.5-12% CBD, 1-5% CBG, 0.1-0.5% c-Eudesmol, 0.4-2% Longifolene, 0.3-1.7% Agarospirol, 0.4-2% Neoisolongifolene, 0.1-0.6% Ledol, 0.5-2.2% Germacrene D, 0.1-0.5% c-Maaliene, 0.2-0.9% bisabolene, 0.05-0.3% caryophyllene oxide and/or 0.05-0.4% Longifolenaldehyde.

According to specific embodiments, the total concentration THC, THCA, CBN, β-caryophyllene, CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde, in the at least two of said liquid chromatography fractions or said compositions is

14-43% THC, 13-40% THCA, 0.5-1.5% CBN, 0-0.05 β-caryophyllene, 0.5-2.2% CBC, 2.5-12% CBD, 1-5% CBG, said 0.1-0.5% c-Eudesmol, 0.4-2% Longifolene, 0.3-1.7% Agarospirol, 0.4-2% Neoisolongifolene, 0.1-0.6% Ledol, 0.5-2.2% Germacrene D, 0.1-0.5% c-Maaliene, 0.2-0.9% bisabolene, 0.05-0.3% caryophyllene oxide and 0.05-0.4% Longifolenaldehyde.

According to specific embodiments, a total concentration of said THC, said THCA, said CBN, said β-caryophyllene, said CBC, said CBD, said CBG said c-Eudesmol, said Longifolene, said Agarospirol, said Neoisolongifolene, said Ledol, said Germacrene D, said c-Maaliene, said bisabolene, said caryophyllene oxide and said Longifolenaldehyde in said at least two of said liquid chromatography fractions or said compositions is as listed in Table 10±10%.

According to specific embodiments, a total concentration of said THC, said THCA, said CBN, said β-caryophyllene, said CBC, said CBD, said CBG said c-Eudesmol, said Longifolene, said Agarospirol, said Neoisolongifolene, said Ledol, said Germacrene D, said c-Maaliene, said bisabolene, said caryophyllene oxide and said Longifolenaldehyde in said at least two of said liquid chromatography fractions or said compositions is as listed in Table 10.

As the present invention have generated a novel composition comprising fractions D6 and D2, according to an alternative or an additional aspect of the present invention, there is provided a composition comprising at least two cannabinoids selected from the group consisting of cannabidiol (CBD), cannabichromene (CBC), cannabigerol (CBG), tetrahydrocannabinol (THC), tetrahydrocannabinolic acid (THCA) and cannabinol (CBN), wherein a concentration of said CBD, said CBC, said CBG, said THC, said THCA and/or said CBN in said composition is 2.5-12% CBD, 0.5-2.2% CBC, 1-5% CBG, 14-43% THC, 13-40% THCA and/or 0.5-1.5% CBN, hereinafter referred to as composition (vi).

According to an alternative or an additional aspect of the present invention, there is provided a composition comprising at least one cannabinoid selected from the group consisting of cannabidiol (CBD), cannabichromene (CBC), cannabigerol (CBG), tetrahydrocannabinol (THC), tetrahydrocannabinolic acid (THCA) and cannabinol (CBN), and at least one cannabinoid selected from the group consisting of c-Eudesmol, Longifolene, Agarospirol, Neoisolongifolene, Ledol, Germacrene D, c-Maaliene, bisabolene, caryophyllene oxide, Longifolenaldehyde and β-caryophyllene,

wherein a concentration of said THC, said THCA, said CBN, said β-caryophyllene, said CBC, said CBD, said CBG said c-Eudesmol, said Longifolene, said Agarospirol, said Neoisolongifolene, said Ledol, said Germacrene D, said c-Maaliene, said bisabolene, said caryophyllene oxide and/or said Longifolenaldehyde.

in said composition is 14-43% THC, 13-40% THCA, 0.5-1.5% CBN, 0-0.05% (3-caryophyllene, 0.5-2.2% CBC, 2.5-12% CBD, 1-5% CBG, 0.1-0.5% c-Eudesmol, 0.4-2% Longifolene, 0.3-1.7% Agarospirol, 0.4-2% Neoisolongifolene, 0.1-0.6% Ledol, 0.5-2.2% Germacrene D, 0.1-0.5% c-Maaliene, 0.2-0.9% bisabolene, 0.05-0.3% caryophyllene oxide and/or 0.05-0.4% Longifolenaldehyde; also referred to hereinafter as composition (vi).

According to specific embodiments, composition (vi) comprises less than 50% tetrahydrocannabinolic acid (THCA).

According to specific embodiments, composition (vi) comprises ≤40% THCA.

According to specific embodiments, composition (vi) comprises components as listed in Table 10, hereinbelow.

According to specific embodiments, composition (vi) comprises at least two, at least three, at least four, at least five, at least six, at least seven, at least eight, at least 9 or at least 10 components as listed in Table 10 hereinbelow.

According to specific embodiments, the concentration of CBD, CBC, CBG, THC, THCA and/or CBN in composition (vi) is as listed in Table 10±10%.

According to specific embodiments, the concentration of CBD, CBC, CBG, THC, THCA and/or CBN in composition (vi) is as listed in Table 10.

According to specific embodiments, the concentration of THC, THCA, CBN, β-caryophyllene, CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde, in composition (vi) is as listed in Table 10±10%.

According to specific embodiments, the concentration of THC, THCA, CBN, β-caryophyllene, CBC, CBD, CBG, c-Eudesmol, Longifolene, Agarospirol, Neoisolongifolene, Ledol, Germacrene D, c-Maaliene, bisabolene, caryophyllene oxide and Longifolenaldehyde in composition (vi) is as listed in Table 10.

According to specific embodiments, composition (vi) comprises a combination of fractions D2 and D6 as shown in Tables 8-10 hereinbelow.

According to specific embodiments, the fraction or the composition disclosed herein has a cytotoxic activity on cutaneous T cell lymphoma (CTCL) cells.

According to specific embodiments, the fraction of the composition has a combined synergistic cytotoxic activity on CTCL as compared to each of the cannabinoids they comprise when administered as a single agent.

Method of determining cytotoxicity are well known in the art and include in-vivo, in-vitro or ex-vivo assays as further described hereinabove and below.

Since the fractions and composition of specific embodiments of the present invention have cytotoxic activity, they may be used for treating malignant diseases.

Thus, according to an aspect of the present invention, there is provided a method of treating an inflammatory disease in a subject in need thereof, the method comprising administering to the subject a therapeutically effective amount of the composition or the article of manufacture disclosed herein, thereby treating the inflammatory disease in the subject.

According to an additional or alternative aspect of the present invention, there is provided the composition or the article of manufacture disclosed herein for use in treating an inflammatory disease in a subject in need thereof.

According to an additional or alternative aspect of the present invention, there is provided a method of treating an inflammatory disease in a subject in need thereof, the method comprising administering to the subject a therapeutically effective amount of a liquid chromatography fraction of a cannabis extract selected from the group consisting of:

(i) a liquid chromatography fraction comprising 40-60 tetrahydrocannabinol (THC);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising cannabidiol (CBD), wherein said CBD is at least 90% CBD and/or wherein said liquid chromatography fraction is devoid of tetrahydrocannabinolic acid (THCA), thereby treating the inflammatory disease in the subject.

According to an additional or alternative aspect of the present invention, there is provided a liquid chromatography fraction of a cannabis extract selected from the group consisting of:

(i) a liquid chromatography fraction comprising 40-60% tetrahydrocannabinol (THC);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising cannabidiol (CBD), wherein said CBD is at least 90% CBD and/or wherein said liquid chromatography fraction is devoid of tetrahydrocannabinolic acid (THCA),

for use in treating an inflammatory disease.

According to an additional or alternative aspect of the present invention, there is provided a method of treating an inflammatory disease in a subject in need thereof, the method comprising administering to the subject a therapeutically effective amount of the composition or the article of manufacture disclosed herein, thereby treating the inflammatory disease in the subject.

According to an additional or alternative aspect of the present invention, there is provided the composition or the article of manufacture disclosed herein for use in treating an inflammatory disease in a subject in need thereof.

According to an additional or alternative aspect of the present invention, there is provided a method of treating an inflammatory disease in a subject in need thereof, the method comprising administering to the subject a therapeutically effective amount of a liquid chromatography fraction of a cannabis extract selected from the group consisting of:

(i) a liquid chromatography fraction comprising 40-60 tetrahydrocannabinol (THC);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fraction is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising at least 90% cannabidiol (CBD) and at least one cannabinoid other than said CBD,

thereby treating the inflammatory disease in the subject.

According to an additional or alternative aspect of the present invention, there is provided a liquid chromatography fraction of a cannabis extract selected from the group consisting of:

(i) a liquid chromatography fraction comprising 40-60% tetrahydrocannabinol (THC);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fraction is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA), and wherein said liquid chromatography fraction comprises said CBD said liquid chromatography fraction comprises at least two of said cannabinoids; and

(iv) a liquid chromatography fraction comprising at least 90% cannabidiol (CBD) and at least one cannabinoid other than said CBD,

for use in treating an inflammatory disease.

According to an additional or alternative aspect of the present invention, there is provided a method of treating an inflammatory disease in a subject in need thereof, the method comprising administering to the subject a therapeutically effective amount of a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; and

(iii) a composition comprising at least two cannabinoids selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA) and said inflammatory disease is not inflammatory bowel disease,

thereby treating the inflammatory disease in the subject.

According to specific embodiments, the method further comprising administering to the subject a therapeutically effective amount of a composition (iv) comprising CBD.

According to an additional or alternative aspect of the present invention, there is provided a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde; and

(iii) a composition comprising at least two cannabinoids selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA) and said inflammatory disease is not inflammatory bowel disease,

for use in a treating an inflammatory disease in a subject in need thereof.

According to specific embodiments, the composition for use further comprises a composition (iv) comprising CBD.

According to an additional or alternative aspect of the present invention, there is provided a method of treating an inflammatory disease in a subject in need thereof, the method comprising administering to the subject a therapeutically effective amount of a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least two cannabinoids selected from the group consisting of cannabidiol (CBD), cannabichromene (CBC) and cannabigerol (CBG), wherein a concentration of said CBD, said CBC and/or said CBG in said composition is 5-30% CBD, 0.1-10% CBC and/or 0.1-15% CBG; and

(iii) a composition comprising at least two cannabinoids selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA) and said inflammatory disease is not inflammatory bowel disease,

thereby treating the inflammatory disease in the subject.

According to specific embodiments, the method further comprising administering to the subject a therapeutically effective amount of a composition (iv) comprising CBD.

According to an additional or alternative aspect of the present invention, there is provided a composition selected from the group consisting of:

(i) a composition comprising 40-60% tetrahydrocannabinol (THC) and at least one cannabinoid selected from the group consisting of tetrahydrocannabinolic acid (THCA), cannabinol (CBN) and β-caryophyllene;

(ii) a composition comprising at least two cannabinoids selected from the group consisting of cannabidiol (CBD), cannabichromene (CBC) and cannabigerol (CBG), wherein a concentration of said CBD, said CBC and/or said CBG in said composition is 5-30% CBD, 0.1-10% CBC and/or 0.1-15% CBG; and

(iii) a composition comprising at least two cannabinoids selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA) and said inflammatory disease is not inflammatory bowel disease,

for use in a treating an inflammatory disease in a subject in need thereof.

According to specific embodiments, the composition for use further comprises a composition (iv) comprising at least 90% CBD and at least one cannabinoid other than the CBD.

According to an additional or alternative aspect of the present invention, there is provided a method of treating an inflammatory disease in a subject in need thereof, the method comprising administering to the subject a therapeutically effective amount of a composition comprising 50-90% cannabidiol (CBD), 10-40% cannabigerol (CBG) and 0.15-0.5% tetrahydrocannabinol (THC).

According to an additional or alternative aspect of the present invention, there is provided a composition comprising 50-90% cannabidiol (CBD), 10-40% cannabigerol (CBG) and 0.15-0.5% tetrahydrocannabinol (THC), for use in a treating an inflammatory disease in a subject in need thereof.

According to an additional or alternative aspect of the present invention, there is provided a method of treating an inflammatory disease in a subject in need thereof, the method comprising administering to the subject a therapeutically effective amount of a composition comprising cannabidiol (CBD), cannabigerol (CBG) and tetrahydrocannabinol (THC) in molar ratios as listed in Table 5.

According to an additional or alternative aspect of the present invention, there is provided a composition comprising cannabidiol (CBD), cannabigerol (CBG) and tetrahydrocannabinol (THC) in molar ratios as listed in Table 5, for use in a treating an inflammatory disease in a subject in need thereof.

As used herein, the term “subject” or “subject in need thereof” includes mammals, preferably human beings at any age or gender. The subject may be healthy or showing preliminary signs of a pathology, e.g. an inflammatory disease e.g. cancer e.g. cutaneous T cell lymphoma (CTCL). This term also encompasses individuals who are at risk to develop the pathology.

As used herein the term “treating” refers to curing, reversing, attenuating, alleviating, minimizing, suppressing or halting the deleterious effects of a disease or disorder [e.g. cancer e.g. cutaneous T cell lymphoma (CTCL)]. Those of skill in the art will understand that various methodologies and assays can be used to assess the development of a pathology, and similarly, various methodologies and assays may be used to assess the reduction, remission or regression of a pathology (e.g. a malignancy), as discussed below.

According to a specific embodiment, treating is preventing.

Inflammatory diseases—Include, but are not limited to, chronic inflammatory diseases and acute inflammatory diseases.

Inflammatory Diseases Associated with Hypersensitivity

Examples of hypersensitivity include, but are not limited to, Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity and DTH.

Type I or immediate hypersensitivity, such as asthma.

Type II hypersensitivity include, but are not limited to, rheumatoid diseases, rheumatoid autoimmune diseases, rheumatoid arthritis (Krenn V. et al., Histol Histopathol 2000 July; 15 (3):791), spondylitis, ankylosing spondylitis (Jan Voswinkel et al., Arthritis Res 2001; 3 (3): 189), systemic diseases, systemic autoimmune diseases, systemic lupus erythematosus (Erikson J. et al., Immunol Res 1998; 17 (1-2):49), sclerosis, systemic sclerosis (Renaudineau Y. et al., Clin Diagn Lab Immunol. 1999 March; 6 (2):156); Chan O T. et al., Immunol Rev 1999 June; 169:107), glandular diseases, glandular autoimmune diseases, pancreatic autoimmune diseases, diabetes, Type I diabetes (Zimmet P. Diabetes Res Clin Pract 1996 October; 34 Suppl:S125), thyroid diseases, autoimmune thyroid diseases, Graves' disease (Orgiazzi J. Endocrinol Metab Clin North Am 2000 June; 29 (2):339), thyroiditis, spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), myxedema, idiopathic myxedema (Mitsuma T. Nippon Rinsho. 1999 August; 57 (8):1759); autoimmune reproductive diseases, ovarian diseases, ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), repeated fetal loss (Tincani A. et al., Lupus 1998; 7 Suppl 2:S107-9), neurodegenerative diseases, neurological diseases, neurological autoimmune diseases, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83), motor neuropathies (Kornberg A J. J Clin Neurosci. 2000 May; 7 (3):191), Guillain-Barre syndrome, neuropathies and autoimmune neuropathies (Kusunoki S. Am J Med Sci. 2000 April; 319 (4):234), myasthenic diseases, Lambert-Eaton myasthenic syndrome (Takamori M. Am J Med Sci. 2000 April; 319 (4):204), paraneoplastic neurological diseases, cerebellar atrophy, paraneoplastic cerebellar atrophy, non-paraneoplastic stiff man syndrome, cerebellar atrophies, progressive cerebellar atrophies, encephalitis, Rasmussen's encephalitis, amyotrophic lateral sclerosis, Sydeham chorea, Gilles de la Tourette syndrome, polyendocrinopathies, autoimmune polyendocrinopathies (Antoine J C. and Honnorat J. Rev Neurol (Paris) 2000 January; 156 (1):23); neuropathies, dysimmune neuropathies (Nobile-Orazio E. et al., Electroencephalogr Clin Neurophysiol Suppl 1999; 50:419); neuromyotonia, acquired neuromyotonia, arthrogryposis multiplex congenita (Vincent A. et al., Ann N Y Acad Sci. 1998 May 13; 841:482), cardiovascular diseases, cardiovascular autoimmune diseases, atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), granulomatosis, Wegener's granulomatosis, arteritis, Takayasu's arteritis and Kawasaki syndrome (Praprotnik S. et al., Wien Klin Wochenschr 2000 Aug. 25; 112 (15-16):660); anti-factor VIII autoimmune disease (Lacroix-Desmazes S. et al., Semin Thromb Hemost. 2000; 26 (2):157); vasculitises, necrotizing small vessel vasculitises, microscopic polyangiitis, Churg and Strauss syndrome, glomerulonephritis, pauci-immune focal necrotizing glomerulonephritis, crescentic glomerulonephritis (Noel L H. Ann Med Interne (Paris). 2000 May; 151 (3):178); antiphospholipid syndrome (Flamholz R. et al., J Clin Apheresis 1999; 14 (4):171); heart failure, agonist-like β-adrenoceptor antibodies in heart failure (Wallukat G. et al., Am J Cardiol. 1999 Jun. 17; 83 (12A):75H), thrombocytopenic purpura (Moccia F. Ann Ital Med Int. 1999 April-June; 14 (2):114); hemolytic anemia, autoimmune hemolytic anemia (Efremov D G. et al., Leuk Lymphoma 1998 January; 28 (3-4):285), gastrointestinal diseases, autoimmune diseases of the gastrointestinal tract, intestinal diseases, chronic inflammatory intestinal disease (Garcia Herola A. et al., Gastroenterol Hepatol. 2000 January; 23 (1):16), celiac disease (Landau Y E. and Shoenfeld Y. Harefuah 2000 Jan. 16; 138 (2):122), autoimmune diseases of the musculature, myositis, autoimmune myositis, Sjogren's syndrome (Feist E. et al., Int Arch Allergy Immunol 2000 September; 123 (1):92); smooth muscle autoimmune disease (Zauli D. et al., Biomed Pharmacother 1999 June; 53 (5-6):234), hepatic diseases, hepatic autoimmune diseases, autoimmune hepatitis (Manns M P. J Hepatol 2000 August; 33 (2):326) and primary biliary cirrhosis (Strassburg C P. et al., Eur J Gastroenterol Hepatol. 1999 June; 11 (6):595).

Type IV or T cell mediated hypersensitivity, include, but are not limited to, rheumatoid diseases, rheumatoid arthritis (Tisch R, McDevitt H O. Proc Natl Acad Sci USA 1994 Jan. 18; 91 (2):437), systemic diseases, systemic autoimmune diseases, systemic lupus erythematosus (Datta S K., Lupus 1998; 7 (9):591), glandular diseases, glandular autoimmune diseases, pancreatic diseases, pancreatic autoimmune diseases, Type 1 diabetes (Castano L. and Eisenbarth G S. Ann. Rev. Immunol. 8:647); thyroid diseases, autoimmune thyroid diseases, Graves' disease (Sakata S. et al., Mol Cell Endocrinol 1993 March; 92 (1):77); ovarian diseases (Garza K M. et al., J Reprod Immunol 1998 February; 37 (2):87), prostatitis, autoimmune prostatitis (Alexander R B. et al., Urology 1997 December; 50 (6):893), polyglandular syndrome, autoimmune polyglandular syndrome, Type I autoimmune polyglandular syndrome (Hara T. et al., Blood. 1991 Mar. 1; 77 (5):1127), neurological diseases, autoimmune neurological diseases, multiple sclerosis, neuritis, optic neuritis (Soderstrom M. et al., J Neurol Neurosurg Psychiatry 1994 May; 57 (5):544), myasthenia gravis (Oshima M. et al., Eur J Immunol 1990 December; 20 (12):2563), stiff-man syndrome (Hiemstra H S. et al., Proc Natl Acad Sci USA 2001 Mar. 27; 98 (7):3988), cardiovascular diseases, cardiac autoimmunity in Chagas' disease (Cunha-Neto E. et al., J Clin Invest 1996 Oct. 15; 98 (8):1709), autoimmune thrombocytopenic purpura (Semple J W. et al., Blood 1996 May 15; 87 (10):4245), anti-helper T lymphocyte autoimmunity (Caporossi A P. et al., Viral Immunol 1998; 11 (1):9), hemolytic anemia (Sallah S. et al., Ann Hematol 1997 March; 74 (3):139), hepatic diseases, hepatic autoimmune diseases, hepatitis, chronic active hepatitis (Franco A. et al., Clin Immunol Immunopathol 1990 March; 54 (3):382), biliary cirrhosis, primary biliary cirrhosis (Jones D E. Clin Sci (Colch) 1996 November; 91 (5):551), nephric diseases, nephric autoimmune diseases, nephritis, interstitial nephritis (Kelly C J. J Am Soc Nephrol 1990 August; 1 (2):140), connective tissue diseases, ear diseases, autoimmune connective tissue diseases, autoimmune ear disease (Yoo T J. et al., Cell Immunol 1994 August; 157 (1):249), disease of the inner ear (Gloddek B. et al., Ann N Y Acad Sci 1997 Dec. 29; 830:266), skin diseases, cutaneous diseases, dermal diseases, bullous skin diseases, pemphigus vulgaris, bullous pemphigoid and pemphigus foliaceus.

Examples of delayed type hypersensitivity include, but are not limited to, contact dermatitis and drug eruption.

Examples of types of T lymphocyte mediating hypersensitivity include, but are not limited to, helper T lymphocytes and cytotoxic T lymphocytes.

Examples of helper T lymphocyte-mediated hypersensitivity include, but are not limited to, Tₕ1 lymphocyte mediated hypersensitivity and Tₕ2 lymphocyte mediated hypersensitivity.

Autoimmune Diseases

Include, but are not limited to, cardiovascular diseases, rheumatoid diseases, glandular diseases, gastrointestinal diseases, cutaneous diseases, hepatic diseases, neurological diseases, muscular diseases, nephric diseases, diseases related to reproduction, connective tissue diseases and systemic diseases.

Examples of autoimmune cardiovascular diseases include, but are not limited to atherosclerosis (Matsuura E. et al., Lupus. 1998; 7 Suppl 2:S135), myocardial infarction (Vaarala O. Lupus. 1998; 7 Suppl 2:S132), thrombosis (Tincani A. et al., Lupus 1998; 7 Suppl 2:S107-9), Wegener's granulomatosis, Takayasu's arteritis, Kawasaki syndrome (Praprotnik S. et al., Wien Klin Wochenschr 2000 Aug. 25; 112 (15-16):660), anti-factor VIII autoimmune disease (Lacroix-Desmazes S. et al., Semin Thromb Hemost. 2000; 26 (2):157), necrotizing small vessel vasculitis, microscopic polyangiitis, Churg and Strauss syndrome, pauci-immune focal necrotizing and crescentic glomerulonephritis (Noel L H. Ann Med Interne (Paris). 2000 May; 151 (3):178), antiphospholipid syndrome (Flamholz R. et al., J Clin Apheresis 1999; 14 (4):171), antibody-induced heart failure (Wallukat G. et al., Am J Cardiol. 1999 Jun. 17; 83 (12A):75H), thrombocytopenic purpura (Moccia F. Ann Ital Med Int. 1999 April-June; 14 (2):114; Semple J W. et al., Blood 1996 May 15; 87 (10):4245), autoimmune hemolytic anemia (Efremov D G. et al., Leuk Lymphoma 1998 January; 28 (3-4):285; Sallah S. et al., Ann Hematol 1997 March; 74 (3):139), cardiac autoimmunity in Chagas' disease (Cunha-Neto E. et al., J Clin Invest 1996 Oct. 15; 98 (8):1709) and anti-helper T lymphocyte autoimmunity (Caporossi A P. et al., Viral Immunol 1998; 11 (1):9).

Examples of autoimmune rheumatoid diseases include, but are not limited to rheumatoid arthritis (Krenn V. et al., Histol Histopathol 2000 July; 15 (3):791; Tisch R, McDevitt H O. Proc Natl Acad Sci units S A 1994 Jan. 18; 91 (2):437) and ankylosing spondylitis (Jan Voswinkel et al., Arthritis Res 2001; 3 (3): 189).

Examples of autoimmune glandular diseases include, but are not limited to, pancreatic disease, Type I diabetes, thyroid disease, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome. Diseases include, but are not limited to autoimmune diseases of the pancreas, Type 1 diabetes (Castano L. and Eisenbarth G S. Ann. Rev. Immunol. 8:647; Zimmet P. Diabetes Res Clin Pract 1996 October; 34 Suppl:S125), autoimmune thyroid diseases, Graves' disease (Orgiazzi J. Endocrinol Metab Clin North Am 2000 June; 29 (2):339; Sakata S. et al., Mol Cell Endocrinol 1993 March; 92 (1):77), spontaneous autoimmune thyroiditis (Braley-Mullen H. and Yu S, J Immunol 2000 Dec. 15; 165 (12):7262), Hashimoto's thyroiditis (Toyoda N. et al., Nippon Rinsho 1999 August; 57 (8):1810), idiopathic myxedema (Mitsuma T. Nippon Rinsho. 1999 August; 57 (8):1759), ovarian autoimmunity (Garza K M. et al., J Reprod Immunol 1998 February; 37 (2):87), autoimmune anti-sperm infertility (Diekman A B. et al., Am J Reprod Immunol. 2000 March; 43 (3):134), autoimmune prostatitis (Alexander R B. et al., Urology 1997 December; 50 (6):893) and Type I autoimmune polyglandular syndrome (Hara T. et al., Blood. 1991 Mar. 1; 77 (5):1127).

Examples of autoimmune gastrointestinal diseases include, but are not limited to, chronic inflammatory intestinal diseases (Garcia Herola A. et al., Gastroenterol Hepatol. 2000 January; 23 (1):16), celiac disease (Landau Y E. and Shoenfeld Y. Harefuah 2000 Jan. 16; 138 (2):122), colitis, ileitis and Crohn's disease.

Examples of autoimmune cutaneous diseases include, but are not limited to, autoimmune bullous skin diseases, such as, but are not limited to, pemphigus vulgaris, bullous pemphigoid and pemphigus foliaceus.

Examples of autoimmune hepatic diseases include, but are not limited to, hepatitis, autoimmune chronic active hepatitis (Franco A. et al., Clin Immunol Immunopathol 1990 March; 54 (3):382), primary biliary cirrhosis (Jones D E. Clin Sci (Colch) 1996 November; 91 (5):551; Strassburg C P. et al., Eur J Gastroenterol Hepatol. 1999 June; 11 (6):595) and autoimmune hepatitis (Manns M P. J Hepatol 2000 August; 33 (2):326).

Examples of autoimmune neurological diseases include, but are not limited to, multiple sclerosis (Cross A H. et al., J Neuroimmunol 2001 Jan. 1; 112 (1-2):1), Alzheimer's disease (Oron L. et al., J Neural Transm Suppl. 1997; 49:77), myasthenia gravis (Infante A J. And Kraig E, Int Rev Immunol 1999; 18 (1-2):83; Oshima M. et al., Eur J Immunol 1990 December; 20 (12):2563), neuropathies, motor neuropathies (Kornberg A J. J Clin Neurosci. 2000 May; 7 (3):191); Guillain-Barre syndrome and autoimmune neuropathies (Kusunoki S. Am J Med Sci. 2000 April; 319 (4):234), myasthenia, Lambert-Eaton myasthenic syndrome (Takamori M. Am J Med Sci. 2000 April; 319 (4):204); paraneoplastic neurological diseases, cerebellar atrophy, paraneoplastic cerebellar atrophy and stiff-man syndrome (Hiemstra H S. et al., Proc Natl Acad Sci units S A 2001 Mar. 27; 98 (7):3988); non-paraneoplastic stiff man syndrome, progressive cerebellar atrophies, encephalitis, Rasmussen's encephalitis, amyotrophic lateral sclerosis, Sydeham chorea, Gilles de la Tourette syndrome and autoimmune polyendocrinopathies (Antoine J C. and Honnorat J. Rev Neurol (Paris) 2000 January; 156 (1):23); dysimmune neuropathies (Nobile-Orazio E. et al., Electroencephalogr Clin Neurophysiol Suppl 1999; 50:419); acquired neuromyotonia, arthrogryposis multiplex congenita (Vincent A. et al., Ann N Y Acad Sci. 1998 May 13; 841:482), neuritis, optic neuritis (Soderstrom M. et al., J Neurol Neurosurg Psychiatry 1994 May; 57 (5):544) and neurodegenerative diseases.

Examples of autoimmune muscular diseases include, but are not limited to, myositis, autoimmune myositis and primary Sjogren's syndrome (Feist E. et al., Int Arch Allergy Immunol 2000 September; 123 (1):92) and smooth muscle autoimmune disease (Zauli D. et al., Biomed Pharmacother 1999 June; 53 (5-6):234).

Examples of autoimmune nephric diseases include, but are not limited to, nephritis and autoimmune interstitial nephritis (Kelly C J. J Am Soc Nephrol 1990 August; 1 (2):140).

Examples of autoimmune diseases related to reproduction include, but are not limited to, repeated fetal loss (Tincani A. et al., Lupus 1998; 7 Suppl 2:S107-9).

Examples of autoimmune connective tissue diseases include, but are not limited to, ear diseases, autoimmune ear diseases (Yoo T J. et al., Cell Immunol 1994 August; 157 (1):249) and autoimmune diseases of the inner ear (Gloddek B. et al., Ann N Y Acad Sci 1997 Dec. 29; 830:266).

Examples of autoimmune systemic diseases include, but are not limited to, systemic lupus erythematosus (Erikson J. et al., Immunol Res 1998; 17 (1-2):49) and systemic sclerosis (Renaudineau Y. et al., Clin Diagn Lab Immunol. 1999 March; 6 (2):156); Chan O T. et al., Immunol Rev 1999 June; 169:107).

Infectious Diseases

Examples of infectious diseases include, but are not limited to, chronic infectious diseases, subacute infectious diseases, acute infectious diseases, viral diseases, bacterial diseases, protozoan diseases, parasitic diseases, fungal diseases, mycoplasma diseases and prion diseases.

Graft Rejection Diseases

Examples of diseases associated with transplantation of a graft include, but are not limited to, graft rejection, chronic graft rejection, subacute graft rejection, hyperacute graft rejection, acute graft rejection and graft versus host disease.

Allergic Diseases

Examples of allergic diseases include, but are not limited to, asthma, hives, urticaria, pollen allergy, dust mite allergy, venom allergy, cosmetics allergy, latex allergy, chemical allergy, drug allergy, insect bite allergy, animal dander allergy, stinging plant allergy, poison ivy allergy and food allergy.

Cancerous Diseases

Cancers encompassed with specific embodiments of the present invention include any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcino sarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Precancers are well characterized and known in the art (refer, for example, to Berman J J. and Henson D E., 2003. Classifying the precancers: a metadata approach. BMC Med Inform Decis Mak. 3:8). Classes of precancers amenable to treatment via the method of the invention include acquired small or microscopic precancers, acquired large lesions with nuclear atypia, precursor lesions occurring with inherited hyperplastic syndromes that progress to cancer, and acquired diffuse hyperplasias and diffuse metaplasias. Examples of small or microscopic precancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

According to specific embodiments, the inflammatory disease if not an inflammatory bowel disease.

According to specific embodiments, the inflammatory disease is cancer.

According to specific embodiments, the cancer is lymphoma.

According to specific embodiments, the lymphoma is cutaneous T cell lymphoma (CTCL).

Non-limiting Examples of CTCL include Mycosis fungoides (MF), transformed mycosis fungoides, Sézary Syndrome, Lymphomatoide Papulosis, CD30+ cutaneous lymphoma and primary cutaneous peripheral T cell lymphoma.

According to specific embodiments, the CTCL is Mycosis fungoides (MF).

According to specific embodiments, the CTCL is Sézary Syndrome.

Hence, according to an aspect of the present invention, there is provided a method of treating a cutaneous T cell lymphoma (CTCL) in a subject in need thereof, the method comprising administering to the subject a therapeutically effective amount of a liquid chromatography fraction of a cannabis extract selected from the group consisting of:

(i) a liquid chromatography fraction comprising 40-60% tetrahydrocannabinol (THC) and/or 35-55% tetrahydrocannabinolic acid (THCA);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA); and

(iv) a liquid chromatography fraction comprising cannabidiol (CBD),

thereby treating the CTCL in the subject.

According to an additional or alternative aspect of the present invention, there is provided a liquid chromatography fraction of a cannabis extract selected from the group consisting of:

(i) a liquid chromatography fraction comprising 40-60% tetrahydrocannabinol (THC) and/or 35-55% tetrahydrocannabinolic acid (THCA);

(ii) a liquid chromatography fraction comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA); and

(iv) a liquid chromatography fraction comprising cannabidiol (CBD),

for use in treating a cutaneous T cell lymphoma (CTCL) in a subject in need thereof.

According to an additional or alternative aspect of the present invention, there is provided a method of treating a cutaneous T cell lymphoma (CTCL) in a subject in need thereof, the method comprising administering to the subject a therapeutically effective amount of a liquid chromatography fraction of a cannabis extract selected from the group consisting of:

(i) a liquid chromatography fraction comprising 40-60% tetrahydrocannabinol (THC) and/or 35-55% tetrahydrocannabinolic acid (THCA);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fraction is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA); and

(iv) a liquid chromatography fraction comprising at least 90% cannabidiol (CBD) and at least one cannabinoid other than said CBD,

thereby treating the CTCL in the subject.

According to an additional or alternative aspect of the present invention, there is provided a therapeutically effective amount of a liquid chromatography fraction of a cannabis extract selected from the group consisting of:

(i) a liquid chromatography fraction comprising 40-60% tetrahydrocannabinol (THC) and/or 35-55% tetrahydrocannabinolic acid (THCA);

(ii) a liquid chromatography fraction comprising at least two cannabinoids selected from the group consisting of cannabidiol (CBD), cannabichromene (CBC) and cannabigerol (CBG), wherein a concentration of said CBD, said CBC and/or said CBG in said liquid chromatography fraction is 5-30% CBD, 0.1-10% CBC and/or 0.1-15% CBG;

(iii) a liquid chromatography fraction comprising at least one cannabinoid selected from the group consisting of cannabidiol (CBD), cannabichromene (CBC), tetrahydrocannabinol (THC) and cannabigerol (CBG), wherein said liquid chromatography fraction comprises at least % CBG and/or wherein said liquid chromatography fraction is devoid of tetrahydrocannabinolic acid (THCA); and

(iv) a liquid chromatography fraction comprising at least 90% cannabidiol (CBD) and at least one cannabinoid other than said CBD,

for use in treating a cutaneous T cell lymphoma (CTCL) in a subject in need thereof.

According to an additional or alternative aspect of the present invention, there is provided a method of treating cutaneous T cell lymphoma (CTCL) in a subject in need thereof, the method comprising administering to the subject a therapeutically effective amount of a composition selected from the group consisting of:

(i) a composition comprising 40-60% tetrahydrocannabinol (THC) and/or 35-55% tetrahydrocannabinolic acid (THCA);

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a composition comprising at least one cannabinoid selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA); and

(iv) a composition comprising cannabidiol (CBD),

thereby treating the CTCL in the subject.

According to an additional or alternative aspect of the present invention, there is provided a composition selected from the group consisting of:

(i) a composition comprising 40-60% tetrahydrocannabinol (THC) and/or 35-55% tetrahydrocannabinolic acid (THCA);

(ii) a composition comprising at least one cannabinoid selected from the group consisting of cannabidiol (CBD), cannabichromene (CBC) and cannabigerol (CBG) and at least one cannabinoid selected from the group consisting of c-Eudesmol, Longifolene, Agarospirol, Neoisolongifolene, Ledol, Germacrene D, c-Maaliene, bisabolene, caryophyllene oxide and Longifolenaldehyde;

(iii) a composition comprising at least one cannabinoid selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA); and

(iv) a composition comprising cannabidiol (CBD),

for use in treating cutaneous T cell lymphoma (CTCL) in a subject in need thereof.

According to an additional or alternative aspect of the present invention, there is provided a method of treating cutaneous T cell lymphoma (CTCL) in a subject in need thereof, the method comprising administering to the subject a therapeutically effective amount of a composition selected from the group consisting of:

(i) a composition comprising 40-60% tetrahydrocannabinol (THC) and/or 35-55% tetrahydrocannabinolic acid (THCA);

(ii) a composition comprising at least two cannabinoids selected from the group consisting of cannabidiol (CBD), cannabichromene (CBC) and cannabigerol (CBG), wherein a concentration of said CBD, said CBC and/or said CBG in said composition is 5-30% CBD, 0.1-10% CBC and/or 0.1-15% CBG;

(iii) a composition comprising at least one cannabinoid selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA); and

(iv) a composition comprising at least 90% cannabidiol (CBD) and at least one cannabinoid other than said CBD,

thereby treating the CTCL in the subject.

According to an additional or alternative aspect of the present invention, there is provided a composition selected from the group consisting of:

(i) a composition comprising 40-60% tetrahydrocannabinol (THC) and/or 35-55% tetrahydrocannabinolic acid (THCA);

(ii) a composition comprising at least two cannabinoids selected from the group consisting of cannabidiol (CBD), cannabichromene (CBC) and cannabigerol (CBG), wherein a concentration of said CBD, said CBC and/or said CBG in said composition is 5-30% CBD, 0.1-10% CBC and/or 0.1-15% CBG;

(iii) a composition comprising at least one cannabinoid selected from the group consisting of cannabidiol (CBD), cannabichromene (CBC), tetrahydrocannabinol (THC) and cannabigerol (CBG), wherein said composition comprises at least 50% CBG and/or wherein said composition is devoid of tetrahydrocannabinolic acid (THCA); and

(iv) a composition comprising at least 90% cannabidiol (CBD) and at least one cannabinoid other than said CBD,

for use in treating cutaneous T cell lymphoma (CTCL) in a subject in need thereof. According to specific embodiments, the compositions and fractions can be used alone or in combination with other established or experimental therapeutic regimen to treat inflammatory disease e.g. cancer, e.g. CTCL.

Thus, for example, the compositions described herein may be administered in conjunction with chemotherapy, radiation therapy, hormonal therapy, targeted therapy, immunotherapy or surgical therapy. Such anti-cancer therapies and methods of utilizing same are well known to one of skill in the art.

Exemplary anti-cancer drugs that can be co-administered with the composition or fractions of the invention include, but are not limited to, Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-Ia; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Taxol; Tecogalan Sodium; Tegafur; Teloxantrone Hydrochloride; Temoporfin; Teniposide; Teroxirone; Testolactone; Thiamiprine; Thioguanine; Thiotepa; Tiazofuirin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

Exemplary anti-CTCL drugs include, but are not limited to, interferon-α, oral bexarotene or other retinoids, extracorporeal photopheresis, antifolates (methotrexate, pralatrexate), histone deacetylase inhibitors such as vorinostat and romidepsin, alemtuzumab, liposomal doxorubicin, gemcitabine, brentuximab vedotin and mogamulizumab.

Any of the above described agents may be administered individually or in combination.

According to specific embodiments, the compositions and fractions are not administered to the subject in combination with an IL6 antagonist or an anti-IL6 antibody.

According to specific embodiments, the compositions and fractions are not administered to the subject in combination with angiogenin.

According to specific embodiments, the compositions and fractions administered to a subject comprise native cannabinoids.

According to specific embodiments, the compositions and fractions administered to a subject do comprise synthetic analogs and derivative of cannabinoids.

Each of the compositions and fractions described herein can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the cannabis derived active ingredients accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For transdermal administration, the composition can be formulated in a form of a gel, a cream, an ointment, a paste, a lotion, a milk, a suspension, an aerosol, a spray, a foam, a serum, a swab, a pledget, a pad or a patch. Formulations for transdermal delivery can typically include carriers such as water, liquid alcohols, liquid glycols, liquid polyalkylene glycols, liquid esters, liquid amides, liquid protein hydrolysates, liquid alkylated protein hydrolysates, liquid lanolin, lanolin derivatives, glycerin, mineral oil, silicone, petroleum jelly, lanolin, fatty acids, vegetable oils, parabens, waxes, and like materials commonly employed in topical compositions. Various additives, known to those skilled in the art, may be included in the transdermal formulations of the invention. For example, solvents may be used to solubilize certain active ingredients substances. Other optional additives include skin permeation enhancers, opacifiers, anti-oxidants, gelling agents, thickening agents, stabilizers, and the like.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran.

Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (cannabis derived active ingredients) effective to prevent, alleviate or ameliorate symptoms of a disorder (e.g., inflammatory disease, e.g. cancer, e.g. CTCL) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Animal models for cancer are described for example in Cheon and Orsulic, Annu. Rev. Pathol. (2011) 6:95-119 and in Abate-Shen, Clinic. Cancer. Res. (2006) 12(18).

An animal model for CTCL is e.g. a human xenograft mouse model described in Krejsgaard T et al. Exp Dermatol. 2010; 19(12):1096-102.

According to one embodiment, the therapeutically effective amount is 0.1-10000 mg/m², 1-1000 mg/m², 10-100 mg/m² or 40-80 mg/m². Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The compositions and fractions can be administered to a subject (e.g., a human) in need thereof in a variety of other forms including a nutraceutical composition.

As used herein, a “nutraceutical composition” refers to any substance that may be considered a food or part of a food and provides medical or health benefits, including the prevention and treatment of disease. In some embodiments, a nutraceutical composition is intended to supplement the diet and contains at least one or more of the following ingredients: a vitamin; a mineral; an herb; a botanical; a fruit; a vegetable; an amino acid; or a concentrate, metabolite, constituent, or extract of any of the previously mentioned ingredients; and combinations thereof.

In some embodiments, a nutraceutical composition of the present invention can be administered as a “dietary supplement,” as defined by the U.S. Food and Drug Administration, which is a product taken by mouth that contains a “dietary ingredient” such as, but not limited to, a vitamin, a mineral, an herb or other botanical, an amino acid, and substances such as an enzyme, an organ tissue, a glandular, a metabolite, or an extract or concentrate thereof.

Non-limiting forms of nutraceutical compositions of the present invention include: a tablet, a capsule, a pill, a softgel, a gelcap, a liquid, a powder, a solution, a tincture, a suspension, a syrup, or other forms known to persons of skill in the art. A nutraceutical composition can also be in the form of a food, such as, but not limited to, a food bar, a beverage, a food gel, a food additive/supplement, a powder, a syrup, and combinations thereof.

Specific embodiments of the present invention also contemplates methods of personalized medicine for CTCL using cannabis-derived medicament.

Hence, according to an aspect of the present invention, there is provided a method of determining a cytotoxic activity of the fraction or composition disclosed herein, the method comprising ex-vivo contacting cutaneous T cells lymphoma (CTCL) cells of a subject with the composition or the fraction, wherein a decrease in viability of said CTCL cells above a predetermined threshold as compared to same in the absence of the composition or the fraction is indicative of the cytotoxic activity of the composition or the fraction.

According to an additional or alternative aspect of the present invention, there is provided a method of determining responsiveness of a subject having cutaneous T cell lymphoma (CTCL) to a cannabis-derived medicament, the method comprising:

(a) ex-vivo contacting CTCL cells of the subject with the cannabis-derived medicament;

(b) determining viability of said CTCL cells following said (a), wherein a decrease in viability above a predetermined threshold as compared to same in the absence of said cannabis-derived medicament is indicative of responsiveness to said cannabis-derived medicament.

According to specific embodiments, the cannabis-derived medicament is a cannabis extract.

According to specific embodiments, the cannabis-derived medicament is purified from cannabis.

According to specific embodiments, the cannabis-derived medicament is a liquid chromatography fraction of a cannabis extract.

According to specific embodiments, the cannabis-derived medicament comprises the composition or the fraction disclosed herein.

According to specific embodiments, the cannabis-derived medicament is synthetic.

According to an additional or an alternative aspect of the present invention, there is provided a method of treating cutaneous T cell lymphoma (CTCL) in a subject in need thereof, the method comprising:

(a) ex-vivo contacting CTCL cells of the subject with at least one liquid chromatography fraction of a cannabis extract;

(b) determining viability of said CTCL cells following said (a); and

(c) administering to said subject a therapeutically effective amount of a liquid chromatography fraction or a combination of liquid chromatography fractions that induced a decrease in viability above a predetermined threshold as compared to same in the absence of said liquid chromatography fraction or said combination of liquid chromatography fractions,

thereby treating the CTCL in the subject.

According to an additional or alternative aspect of the present invention, there is provided a method of treating cutaneous T cell lymphoma (CTCL) in a subject in need thereof, the method comprising:

(a) ex-vivo contacting CTCL cells of the subject with at least one liquid chromatography fraction of a cannabis extract;

(b) determining viability of said CTCL cells following said (a); and

(c) selecting for treatment a liquid chromatography fraction or a combination of liquid chromatography fractions that induced a decrease in viability above a predetermined threshold as compared to same in the absence of said liquid chromatography fraction or said combination of liquid chromatography fractions,

thereby treating the CTCL in the subject.

According to specific embodiments, the at least one liquid chromatography fraction of a cannabis extract comprises the composition or the fraction disclosed herein.

Method of determining viability or cytotoxicity are well known in the art and include, but not limited to XTT viability assay, Alamar Blue and/or Cell sorting (e.g. for annexin V).

According to specific embodiments, the viability or cytotoxicity is determined by XTT viability assay.

According to one embodiment, a predetermined threshold can be established by determining viability of healthy cells or a tissue (e.g. of a healthy donor subject, of the subject before disease onset or during disease remission, or from tissue cultures available commercially).

According to specific embodiments, the predetermined threshold is a significant decrease in viability as compared to same in the absence of the composition, fraction or a cannabis-derived medicament as determined by e.g. XTT viability assay.

According to specific embodiments, the predetermined threshold is a decrease of at least 5%, at least 10%, 20%, 30%, 40% or even higher say, 50%, 60%, 70%, 80%, 90%, 99% or even 100% as compared to same in the absence of the composition, fraction or a cannabis-derived medicament as determined by e.g. XTT viability assay.

According to specific embodiments the predetermined threshold is at least 1.5 fold, at least 2 fold, at least 3 fold, at least 5 fold, at least 10 fold, or at least 20 fold as compared to same in the absence of the composition, fraction or a cannabis-derived medicament (as further described hereinbelow) as determined by e.g. XTT viability assay.

According to specific embodiments, the CTCL cells of the subject are obtained by biopsy.

As used herein, a % of a cannabinoid in the fractions and compositions disclosed herein refers to the % calculated from concentration (w/v) of the recited cannabinoid out of the total cannabinoids, active ingredients or compounds in the fraction or composition, as determined by the peak area according to a GC/MS profile of the fraction or composition.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Extraction of Cannabis Inflorescence

Fresh inflorescences of Cannabis sativa (C. sativa) strain DQ and dry inflorescences of C. sativa strain SCBD (IMC, Israeli Medical Cannabis, Israel), were harvested from plants (by IMC), taken immediately for extraction and frozen at −20° C. Following, the Cannabis inflorescences were pulverized with liquid nitrogen by pestle and mortar. Absolute ethanol was added to each tube containing the powder at a sample-to-absolute ethanol ratio of 1:4 (w/v). The tubes were mixed thoroughly on a shaker for 30 minutes and then the extract was filtered through a filter paper (0.2 PVDF syringe filter) by vacuum filtration. Following, the filtrate was transferred to new tubes and the solvent was evaporated under nitrogen. The dried extract was weighed and resuspended in absolute methanol (volume of the solvent was added according to the desired concentration) and filtered through a 0.45 μm syringe filter. For cells treatments, the resuspended extract was diluted for cell cultures.

Chemical Characterization

Standard preparation—The cannabinoid standards cannabigerol (CBG, Restek catalog no. 34091), cannabidiol (CBD, Restek catalog no. 34011), cannabidiolic acid (CBDA, Restek catalog no. 34099), cannabinol (CBN, Restek catalog no. 34010), cannabigerolic acid (CBGA, Sigma-Aldrich catalog no. C-142), tetrahydrocannabinol (THC, Restek catalog no. 34067), cannabichromene (CBC, Restek catalog no. 34092) and tetrahydrocannabinolic acid (THCA, Restek catalog no. 34093) were diluted with methanol to a concentration of 10 ppm and subjected to HPLC. For quantification of cannabinoid, the standards were dissolved in methanol at different concentrations from 5 ppm to 60 ppm.

Sample preparation—For analytical HPLC, the dry crude extract was resuspended in methanol and filtered through a 0.45 μm syringe filter (Merck, Darmstadt, Germany). Following, 20 μl of the filtered extract were injected to the analytical HPLC (in order to get a profile). For preparative HPLC, 50 mg of the dry crude extract were dissolved in 10 ml solvent (75% MeOH and 25% water having 0.1% acetic acid) and filtered through a 0.45 μm pore size Syringe filter. Following, 10 ml of the filtered extract were injected to the preparative HPLC for separation (fraction collection).

Analytical HPLC analysis—Sample profile was obtained from an UltiMate 3000 HPLC system coupled with WPS-3000(T) Autosampler, HPG-3400 pump, and DAD-300 detector. The separation was performed on a Raptor ARC-18 column, 2.7 μm, 150×4.6 mm (RESTEK, 9314A65). Diluent:water:methanol (1:3, v:v), injection volume was 5 μl. Mobile phase:Solvent gradient was formed by isocratic proportion with 25% solvent A (water with 5 mM ammonium formate and 0.1% formic acid) and 75% solvent B (acetonitrile with 0.1% formic acid) at a flow rate of 1.5 ml/min for total runtime of 10 minutes. The compound peaks were detected at 220 and 280 nm.

Preparative HPLC analysis—Sample separation was effected in an Agilent Technologies 1260 Infinity preparative HPLC system, 1260 MWD-VL detector, Column: Kinetex 5u EVO C18 100A, 250×21.2 mm (Phenomenex). Solvent gradients were formed as indicated below:

Prep-HPLC method of sCBD line:

| Time | Sol A [0.1% Acetic acid] (in %) | Sol B [100% MeOH] (in %) |
|---|---|---|
| 0 min | 40 | 60 |
| 45 min | 15 | 85 |
| 55 min | 15 | 85 |

Prep-HPLC method of DQ line:

| | | Sol A | Sol B |
|---|---|---|---|
| | | [0.1% Acetic acid] | [100% MeOH] |
| Time | | (in %) | (in %) |
| 0 | min | 25 | 75 |
| 30 | min | 15 | 85 |
| 35 | min | 15 | 85 |

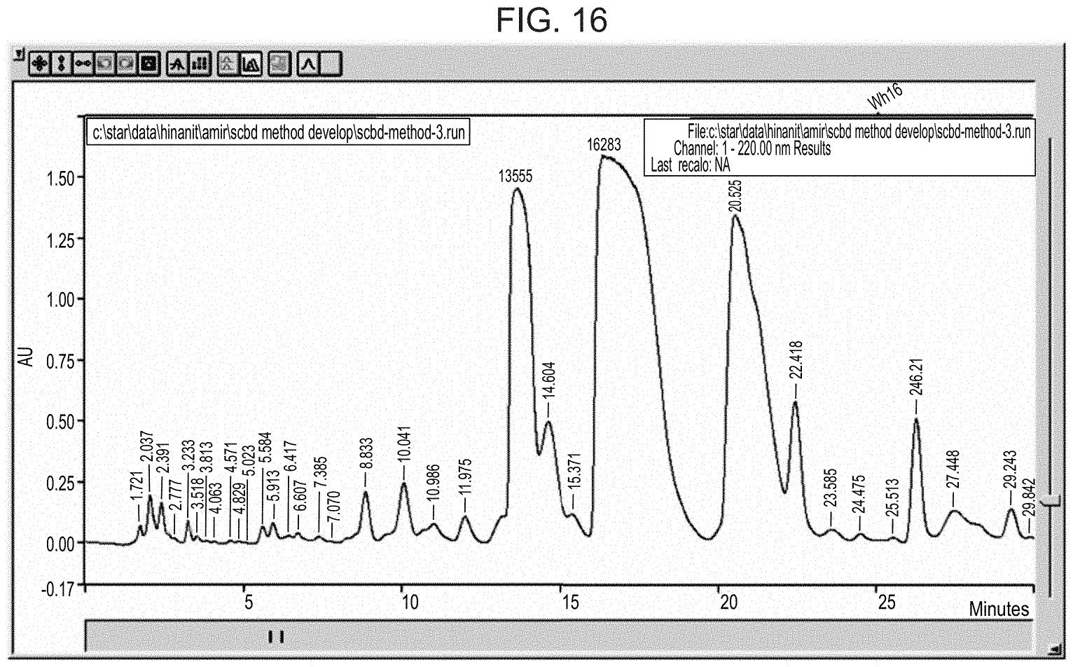

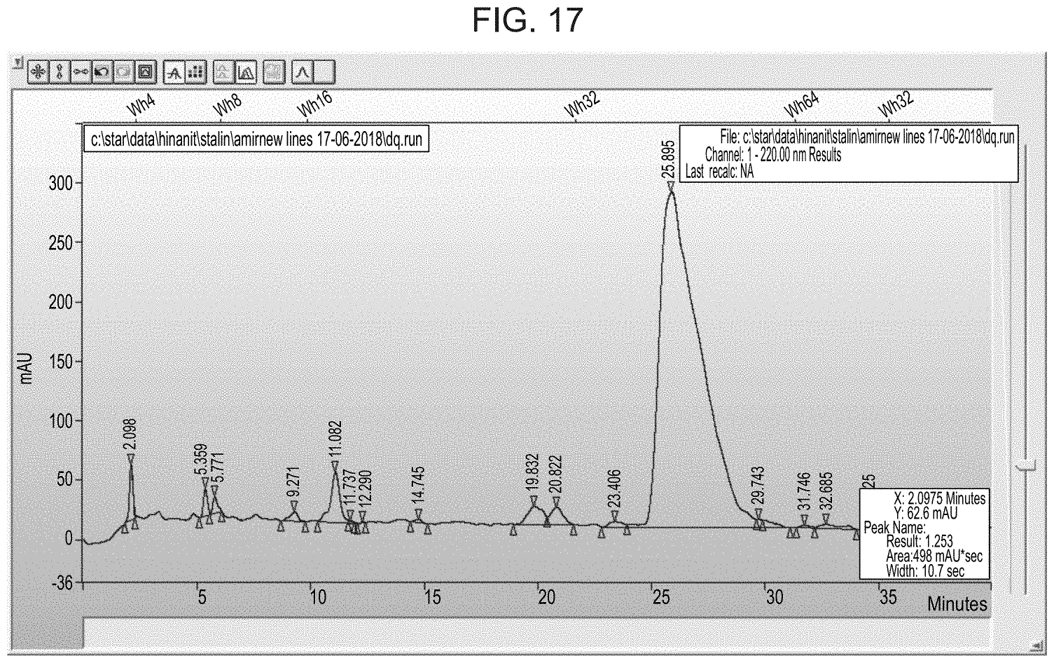

The compound peaks were detected at 220, 240 and 280 nm. The 220-nm peaks were taken for further processing. The extracts were fractionated into nine fractions (for sCBD) and six fractions (for DQ) according to the obtained chromatograms (FIGS. 16-17). Retention times were: D6-21.91-26.42 min, D2-7.62-8.34 min, S5-24.24-25.07 min and S4-22.88-24.23 min.

Gas chromatograph (GC) with mass selective detector (MSD) (GC/MS) analysis—GC/MS analyses were carried out using a HP7890 gas chromatograph coupled to a HP6973 mass spectrometer with electron multiplier potential 2 KV, filament current 0.35 mA, electron energy 70 eV, and the spectra were recorded over the range m/z 40 to 400. An Agilent 7683 autosampler was used for sample introduction. Helium was used as a carrier gas at a constant flow of 1.1 ml s⁻¹. One μl of each sample was injected to the GC/MS using a 1:10 split ratio injection mode. An isothermal hold at 50° C. was kept for 2 minutes, followed by a heating gradient of 6° C. min-1 to 300° C., with the final temperature held for 4 minutes. A 30 m, 0.25 mm ID 5% cross-linked phenylmethyl siloxane capillary column (HP-5MS) with a 0.25 μm film thickness was used for separation and the injection port temperature was 220° C. The MS interface temperature was 280° C. Peak assignments were performed with a spectral library (NIST 14.0) and compared with published and MS data obtained from the injection of standards purchased from LGC standards. Prior to GC/MS analysis, 200 μL of N,O-bis(trimethylsilyl)trifluoroacetamide (BSTFA, Sigma-Aldrich, Israel) containing 1% of trimethylchlorosilane (TMCS) was added to each completely dried extract and heated to 70° C. for 20 minutes. Concentrated fractions were injected to the GC-MS in relative concentrations ˜1000 times higher than those prepared for treatments. For concentration, 1 mg of each sample was dried under a gentle stream of nitrogen, and then dissolved in 6 mL of methanol prior to introduction to GC-MS.

Cell Cultures

MyLa cells [lymphoma cells established from skin biopsies of a patient with Mycosis Fungoides (MF), Cat No: 95051032, ECACC] maintained in Dulbecco's Modified Eagle Medium (DMEM) and HUT78 cells (lymphoma cells established from PBL of a patient with Sezary Syndrome, Cat No: TIB-161, ATCC) maintained in RPMI1640 medium, were grown at 37° C. in a humidified 5% CO₂, 95% air atmosphere.

PBL donors—Samples were collected from 7 Sézary patients who attended the Department of Dermatology, at Rabin Medical Center. All had Sézary by revised staging criteria [Olsen E, et al. Blood. 2007; 110: 1713-22], with clinical stage IVA disease. In addition, blood samples enriched with PBL were obtained from leftover blood of 4 healthy blood donors at Magen David Adom, Sheba Medical Center, Israel. All patients provided their written informed consent to participate in the study, approved by the Ethics Committee of Rabin Medical Center.

Isolation of human peripheral blood lymphocytes—Peripheral blood was diluted 1:2 in sterile phosphate-buffered saline (PBS). Same volume of Lymphoprep (STEMCELL Technologies) was added to the peripheral blood sample with a Pasteur pipette, and the sample was centrifuged (800×g, 20 minutes, 20° C.). Peripheral blood lymphocytes (PBL) were collected from the white median interphase, rinsed twice with PBS, and suspended in RPMI medium with 10% Fetal Bovine Serum (FBS) to 2×10⁶ cells/ml.

Preparation of compositions comprising pure cannabinoids—The phytocannabinoid standards cannabigerol (CBG, Restek catalog no. 34091), cannabidiol (CBD, Restek catalog no. 34011), Δ-9 tetrahydrocannabinol (Δ-9 THC, Restek catalog no. 34067) and cannabichromene (CBC, Restek catalog no. 34092) were received in concentration of 1 mg/ml, originally dissolved in methanol. Work solution was freshly prepared by diluting these phytocannabinoids standards in medium in a ratio of 1:10. Methanol levels werew not allowed to rise above 5% of the final solution.

XTT Cell Viability Assays—Cells were seeded in 96-wells plates at 10,000 cells per well in triplicates in DMEM or RPMI640 growing media. The following day, cells were treated with the different extracts, separated fractions and combinations thereof or pure compounds; or media or solvent only as control. To evaluate involvement of the CB1 and CB2 receptors, 10 μM of AM251 (a CB1 receptor inverse agonist, TOCRIS, 1117/1) or SR144528 (a CB2 receptor inverse agonist, Abcam) were added to the cells 1 hour prior to treatment. Cells were incubated for 48 hours and following XTT (2,3-bis(2-methoxy-4-nitro-5-sulfophenyl)-5-[(phenylamino)-carbonyl]-2H-tetrazolium inner salt) reduction was used to quantify viability according to manufacturer's instruction (BI, Kibbutz Beit-Haemek, Israel). Briefly, cells were incubated with XTT reagent for 2 hours at 37° C. in a humidified 5% CO₂, 95% air atmosphere. Absorbance was recorded by a photometer Synergy H1 hybrid reader (BioTek) at 490 nm with 650 nm of reference wavelength. Cells survival was estimated according to the following equation: % cell survival=100×(At−Ac)(treatment)/(At−Ac)(control), where At and Ac are the absorbencies (490 nm) of the XTT colorimetric reaction in treated and control cultures, respectively, minus non-specific absorption measured at 650 nm. Absorbance of medium alone was also deducted from specific readings.

Analysis of combined drug effects—In order to examine synergy between the fractions (D2 and D6 or S4 and S5), XTT assay was used on MyLa or HUT78 cells as described above. Different concentrations of the fractions were examined as the following: different concentrations of D2 (6 μg/mL to 24 μg/mL), with and without the IC50 dose of D6 (30 μg/mL), different concentrations of D6 (10 μg/mL to 20 μg/mL) with and without the IC50 dose of D2 (48 μg/mL), different concentrations of S4 (5 μg/mL to 20 μg/mL), with and without the IC50 dose of S5 (10 μg/mL) or different concentrations of S5 (4 μg/mL to 12.5 μg/mL) with and without the IC50 dose of S4 (16 μg/mL), were used to treat the cells for 48. Next, the cells were incubated with XTT reagent for 2 hours as described above. Drug synergy was determined by Bliss independence drug interaction model which is defined by the following equation:

Exy=Ex+Ey−(ExEy)

Where (Exy) is the additive effect of the drug x and y as predicted by their individual effects (Ex and Ey). For calculation purpose, the drug's anti-cancer effect was defined as complementary to the obtained results (1-Exy). The observed combined percentage viability is then compared to the calculated value. If the observed value of Exy is greater than the calculated Exy value, the combination treatment is considered as worse than expected, which means antagonism effect. If the observed value is less than the calculated one, then the combination treatment is considered as better than expected, thus showing synergism effect. If both values are equal, the combination treatment is considered as the same for the addition of the two drugs, which means additive effect (independent).

Annexin V Apoptosis Assay for CTCL lines—Apoptosis for CTCL cell lines was assessed using MEBCYTO Apoptosis Kit with Annexin V-FITC and PI (MBL, Enco, 4700). Staining was according to manufacturer instructions. Briefly, cells were seeded in 6-well plate culture dishes, at density of 1×10⁶ cells per well in DMEM (for My-La cells) or in RPMI (for HuT-78 cells). The following day, the media was replaced with new media containing the indicated fractions, or methanol as control. Cells were then incubated for 48 hours at 37° C. in a humidified 5% CO₂-95% air atmosphere. Following incubation, cells were harvested and collected separately. Tubes were centrifuged for 10 minutes at 900 g and cell pellets were resuspended and washed twice with 1 mL PBS. The cells in each sample were resuspended in 85 μL of Annexin binding buffer. Cells were stained using 10 μL Annexin V-FITC solution and 5 μL propidium iodide (PI) working solution followed by incubation in the dark at room temperature for 15 minutes. Then 400 μL of Annexin V binding buffer were added to each tube and flow cytometry was performed using a Gallios flow cytometer (FACS). Cells were considered to be apoptotic if they were Annexin V+/PI− (early apoptotic) or Annexin V+/PI+ (late apoptotic). Live cells were defined as Annexin V−/PI−, and Annexin V−/PI+ as necrosis.

Cell cycle analysis for CTCL cell lines—Cells were seeded in 6-wells culture plates at a concentration of 2×10⁵ cells/mL, 10,000 cells per well. Following 24 hours of incubation, cells were treated with S4 [5 μg/M1], S5 [6 μg/mL], combination of S4 and S5, or methanol as control for another 48 hours. Cells from each well were then harvested and collected separately and centrifuged for 10 minutes at 900 g. The cells pellet was washed once with 1 mL of PBS and fixed with 70% cold ethanol at 4° C. for 1 hour. The fixed cells were pelleted out and washed twice with 1 mL of PBS. The cell pellet was stained by resuspending in 250 μL of PI solution (50 μg/mL) containing RNase A (100 μg/mL) for 15 minutes in the dark. Following, 400 μL PBS were added to each tube and the cells were analyzed using FACS.

Apoptosis assay for PBL obtained from Sèzary patients—PBL obtained from Sézary patients (SPBL) (1×10⁶ cells) were washed with PBS followed by binding buffer (BMS-500FI, eBioscience). Cells were suspended in 100 μL binding buffer with 1 μL Annexin V-FITC (4830-01-1, eBioscience)+2 μL CD26 Alexa Fluor 405-conjugated antibody (FAB1180V-100UG, R&D SYSTEMS)+10 μL CD4-APC-conjugated antibody (FAB3791A-100, R&D SYSTEMS) and incubated for 15 minutes at room temperature. Cells were washed with binding buffer and suspended in 190 μL binding buffer+1 μl of PI (00-6990-42, eBioscience). 300 μL PBS were added and samples were analyzed by FACS. The percent of apoptotic cells (annexin positive cells) was determined in CD4⁺CD26⁻ gated lymphocytes and in non-CD4₊CD26⁻ gated lymphocytes. The apoptosis induction of each treatment was obtained by reducing the percent of apoptotic cells treated with the methanol control from the percent of apoptotic cells treated with the fractions.

Gene Expression profiling—MyLa cancer cells were treated with SCBD fractions S4 (5 μg/mL) and S5 (μg/mL) and their combination (i.e. 5 μg/mL S4+5 μg/mL S5) for 4 hours. Following, RNA was extracted using TRI reagent (Sigma-Aldrich) according to the manufacturer's protocol and gene expression was determined using a StepPlus One system (ABI) using the following primers:

IL-13
forward
(SEQ ID NO: 1)
5′-GAGTGTGTTTGTCACCGTTG-3′
and
reverse
(SEQ ID NO: 2)
5′-TACTCGTTGGTCGAGAGCTG-3′;
AKT1
forward
(SEQ ID NO: 3)
5′-GCTCACCCAGTGACAACTCA-3′
and
reverse
(SEQ ID NO: 4)
5′-CCCAGCAGCTTCAGGTACTC-3′;
SATB1
forward
(SEQ ID NO: 5)
5′-TGGTAAACCTTCGGGCTATG-3′
and
reverse
(SEQ ID NO: 6)
5′-CCATTCCTTTCAGTGGCAAT-3′;
RRM2-9
forward
(SEQ ID NO: 7)
5′-CCTCAGGTGACCTCTCCAAG-3′
and
reverse
(SEQ ID NO: 8)
5′-TACTATGCCATCGCTTGCTG-3′;
NFKBIZ-1
forward
(SEQ ID NO: 9)
5′-GGCAGCTGAAGAAGCAAATC-3′
and
reverse
(SEQ ID NO: 10)
5′-TCAACCGATACTGCAAGCTG-3′;
KCNN4
forward
(SEQ ID NO: 11)
5′-CATCACATTCCTGACCATCG-3′
and
reverse
(SEQ ID NO: 12)
5′-ACGTGCTTCTCTGCCTTGTT-3′;
PIK3R3-1
forward
(SEQ ID NO: 13)
5′-AGCCTGTGGAAATGGCATAG-3′
and
reverse
(SEQ ID NO: 14)
5′-CTCTCATGAAGGAGGCCAAG-3′;
ATF-4
forward
(SEQ ID NO: 15)
5′-GGAAACCATGCCAGATGACC-3′
and
reverse
(SEQ ID NO: 16)
5′-ACTTTCTGGGAGATGGCCAA-3′;
TRIB-3
forward
(SEQ ID NO: 17)
5′-GGTGCTTATCAGGTGCCAAG-3′
and
reverse
(SEQ ID NO: 18)
5′-GTTGTCAGCTCAAGGATGCC-3′.

RNA sequencing and transcriptome analysis—For RNA preparation, cells were seeded into a 6-well plate at a concentration of 1.5×10⁶ cell/mL per well. Following 24 hours of incubation at 37° C. in a humidified 5% CO₂-95% air atmosphere, cells were treated with S4 (5 μg/mL), S5 (6 μg/mL), a combination of S4 and S5 at these concentrations, and methanol for 6 hours. The cells were subsequently harvested and total RNA was extracted using a TRI reagent (Sigma-Aldrich) according to manufacturer's protocol. The RNA was kept at −80° C. until further analysis. Sequencing libraries were prepared using the INCPM mRNA Seq protocol. Sixty bp single reads were sequenced on one lanes of an Illumina HiSeq. Transcriptome analysis was carried out as described in [Nallathambi R, et al. Cannabis and Cannabinoid Research. 2018; 3: 120-35]. Briefly, the raw-reads were subjected to a filtering and cleaning procedure and FASTX Toolkit (www(dot)hannonlab(dot)cshl(dot)edu/fastx toolkit/index(dot)html, version 0.0.13.2) was used to trim read-end nucleotides with quality scores <30, and to remove reads with less than 70% base pairs with a quality score ≤30, using the FASTQ Quality Filter. Clean-reads were aligned to the human genome extracted from National Center for Biotechnology Information (NCBI) (GRCh38; www(dot)ncbi(dot)nlm(dot)nih(dot)gov/genome/guide/human/) using Tophat2 software (v2.1). Gene abundance estimation was performed using Cufflinks (v2.2) combined with gene annotations from the NCBI. Heatmap visualization was performed using R Bioconductor and differential expression analysis was completed using the DESeq2 R package. Genes that varied from the control more than twofold, with an adjusted P-value of no more than 0.05, were considered differentially expressed. The KEGG database (www(dot)genome(dot)jp/kegg/) was used for pathway analysis using the KEGG mapper tool (www(dot)genome(dot)jp/kegg/tool/map_pathway2(dot)html). Enrichr tool was used for pathway enrichment analysis (www(dot)amp(dot)pharm(dot)mssm(dot)edu/Enrichr/).

### Example 1

### Extracts from C. Sativa Strain SCBD Inflorescences are Active in Reducing Cell Viability in Cutaneous T Cell Lymphoma Cell Lines

Cytotoxic activity was determined as the level of cell viability in MyLa cells [lymphoma cells established from skin biopsies of a patient with Mycosis Fungoides (MF)] and HUT78 cells (lymphoma cells established from PBL of a patient with Sezary Syndrome) cells for extracts of dry inflorescences of C. sativa strain SCBD following 48 hours of treatment. Treatment with the extracts reduced MyLa cancer cell viability in a dose dependent manner (FIG. 1). Similarly, treatment with the extracts significantly reduced HUT78 cancer cell viability (FIGS. 6A-B).

Following, the extracts were fractionated by HPLC. Several fractions were collected and high concentrations and each was examined for cytotoxic activity on MyLa cancer cells.

Fractions denoted herein as S4 and S5 were the most active fractions and had a similar activity compared to the whole SCBD extract on MyLa cells, and also to the activity of 300 ng/ml doxorubicin (FIG. 2). Moreover, treatment with each of S4 or S5 reduced MyLa cancer cell viability in a dose dependent manner, with and IC50 of 16.09 μg/ml and 9.72 μg/ml, respectively (FIGS. 3A-B and 4A-B).

Interestingly, a combined treatment with S4 and S5 had a synergistic cytotoxic activity on MyLa and HUT78 cancer cells (FIGS. 5A and 6A-B). Following, the partial effect of the fractions was calculated according to the Bliss independence model for each combination experiment. As shown in Table 1A below the Bliss model calculations and indicated that combinations of S4 and S5 are synergistic.

TABLE 1A
Synergy calculations of cytotoxic activity
(based on Bliss Model) of S4 and S5 combinations on MyLa cells

| | S4 16 μg/ml | | | | |
|---|---|---|---|---|---|
| | S5 10 μg/ml | S5 8 μg/ml | S5 6 μg/ml | S5 4 μg/ml | |
| Calculated value (μg/ml) | 18.14 | 12.44 | 29.2290346 | 65.7992666 | |
| Experimental value (μg/ml) | 10.64 | 10.90 | 12.79 | 16.38 | |
| | S5 10 μg / ml | | | | |
| | S4 20 μg/ml | S4 14 μg/ml | S4 12 μg/ml | S4 10 μg/ml | S4 5 μg/ml |
| Calculated value (μg/ml) | 13.78 | 44.67 | 42.29 | 48.4543202 | 40.5399875 |
| Experimental value (μg/ml) | 9.73 | 9.34 | 10.38 | 9.90 | 18.02 |
| | | S4 5μg/ml | S4 5 μg/ml | S4 4 μg/ml | |
| | | S5 6 μg/ml | S5 2 μg/ml | S5 4 μg/ml | |
| Calculated value (μg/ml) | | 170.30 | 121.04 | 166.89 | |
| Experimental value (μg/ml) | | 22.24 | 133.55 | 62.67 | |

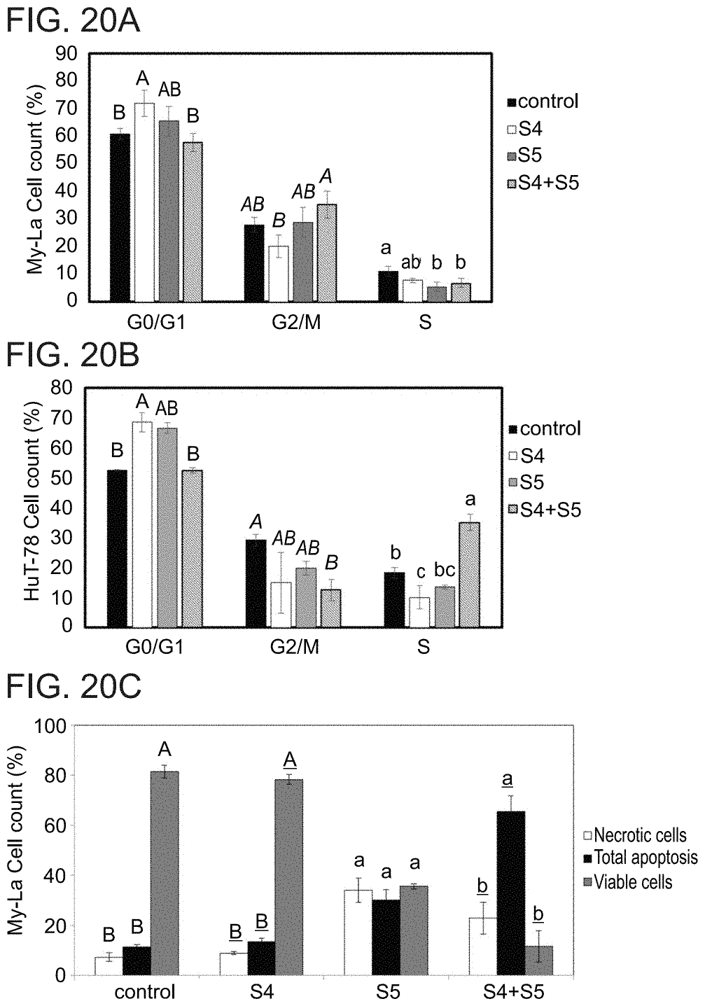

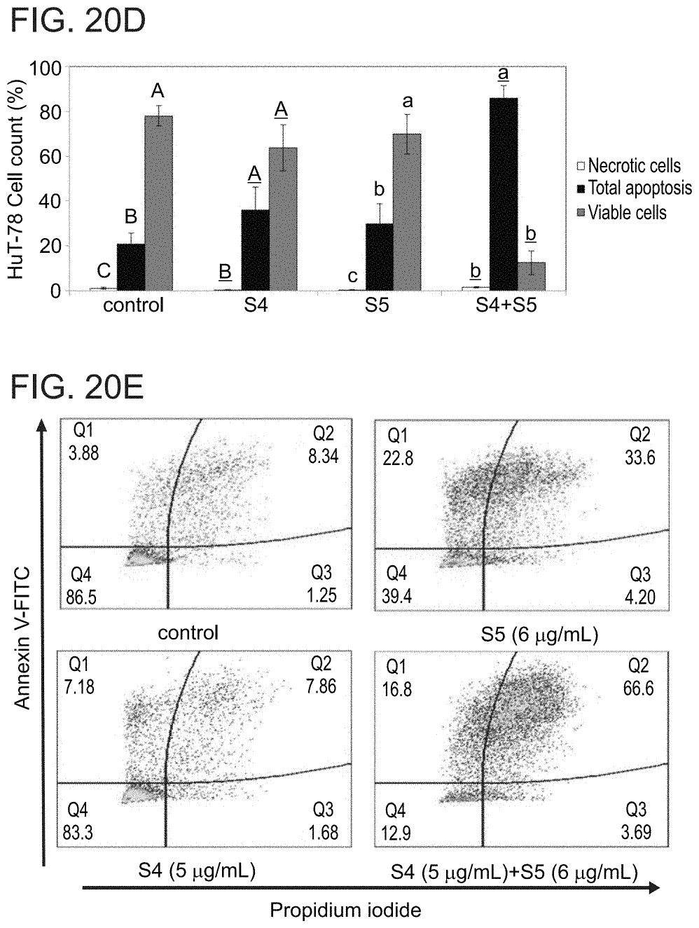

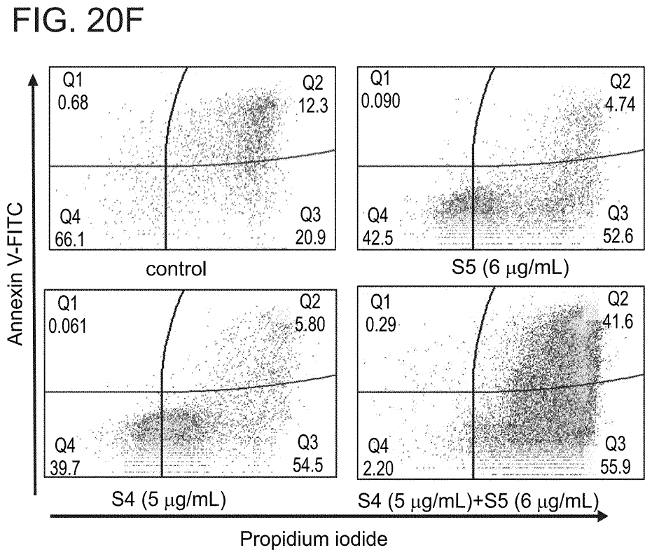

Interestingly, the cytotoxic activity was found to involve cell apoptosis and cell cycle arrest. Combined treatment with S4 and S5 led to an increase in My-La cells that are in the G2-M phase of the cell cycle, in comparison to the control (35.3% vs. 28%, FIG. 20A); and to an increase (30%) in HuT-78 cells that are in the S phase, in comparison to the control (30% vs. 18.3%, FIG. 20B), following 24 hours of treatment. 48 hours of treatment led to an increase in apoptosis in My-La or HuT-78 cells treated with S4 and S5, as compared to the control (65.4% vs. 11.3% and 85.9% vs. 20.9% in My-La or HuT-78 cells respectively, FIGS. 20C-F). The proportion of apoptotic cells in S5-only and S4-only treated cells at 48 hours was higher compared to the control but lower compared to the S4 and S5 combined treatment (FIGS. 20C-F). Interestingly, treatment of My-La cells with S4 lead to a slight increase in apoptotic cell death rates compared to control treatment (FIGS. 20C and 20E).

In the next step the effect of a S4, S5 and their combination on viability of peripheral blood lymphocytes obtained from Sézary patients (SPBL) was determined and were correlated with the effects observed on My-La and HuT-78 cell lines (Table 1B hereinbelow).

TABLE 1B
Synergy calculations of cytotoxic activity (based on Bliss Model)
of S4 and S5 combinations on MyLa cells, HuT-78 cells and SPBLs

| | % cell viability | % cell viability | % cell viability |
|---|---|---|---|
| Treatment | in My-La | in HuT-78 | in SPBL |
| S4 20 μg/mL + S5 10 μg/mL | 6.09 ± 1.51 | 15.74 ± 1.02 | 20.32 ± 7.06 (n = 2) |
| S4 5 μg/mL + S5 6 μg/mL | 37.10 ± 7.72 | 10.38 ± 1.55 | 55.98 ± 4.37 (n = 7) |
| S4 5 μg/mL + S5 2 μg/mL | ±6.91122.19 | ±7.2697.79 | 80.92 ± 8.69 (n = 4) |
| S4 4 μg/mL + S5 4 μg/mL | 68.86 ± 11.24 | 49.34 ± 3.44 | ND |

In the PBL setting as well, the cytotoxic activity was found to involve cell apoptosis (Table 11 hereinbelow and FIGS. 21A-C). SPBL is a population of periphery blood lymphocytes taken from Sezary patients, and as such it contains different limphocytes and a mixture of malignant and healthy T cells. Hence, to assesss the effect of the S4+S5 treatment on the malignant cells only, based on CD4 and CD26 markers, malignant T cells (CD4⁺CD26⁻) were selected from non malignant T cells (non-CD4⁺CD26⁻). As shown in FIG. 21B, the average percentage of apoptosis induction calculated for 4 of the SPBL CD4⁺CD26⁻ cell populations (a marker of Sézary-enriched cells [20]) by each fraction separately was about 2.5%, while a treatment with S4 and S5 fractions led to 53% apoptotic cells, indicating a significant (p=0.0004) increase in apoptosis with the combined treatment. Moreover, the combined treatment led to a higher proportion of apoptotic cells in the SPBL malignant CD4₊CD26⁻ cells sub-population as compared to the non-malignant non-CD4⁺CD26⁻ sub-population (FIGS. 21A and 21C), indicating that the induction of apoptosis is significantly selective to the cancerous cells. Specifically, in the 60.5% CD4⁺CD26⁻ cells present in SPBL, apoptosis induction was 65%; whereas in the 39.5% non-CD4⁺CD26⁻ cells present in SPBL, apoptosis induction was 31% (p=0.0016).

In addition, a significant increase in mRNA expression of IL-13 in MyLa cancer cells was detected following treatment with S4 and a synergistic effect on IL-13 mRNA expression was noted with a combined treatment with S4 and S5 (FIG. 18), suggesting that combined S4 and S5 treatment leads to synergy also in relation to gene expression. Consequently, in order to identify genes and pathways differentially expressed in My-La and HuT-78 following treatment with the purified fractions and their combination, RNA sequence analysis of cells treated with S4 [5 μg/mL], S5 [6 μg/mL], and their combination was effected. Sample correlation tests showed that RNA sequencing resulted in separate clusters for My-La vs. HuT-78 cells (FIG. 22A). In each of the cell lines, results from cells treated with S4 or S5 were clustered together, and those of the control treatment clustered in a separate Glade (FIG. 22A). However, the cell lines treated with a combination of S4 and S5 clustered as an outgroup Glade separate from the rest of the treatments (FIG. 22A). My-La and HuT-78 cells treated with the combination of S4 and S5 resulted in 3716 and 2935 genes, respectively, differentially expressed genes compared to the control and to cells treated with S4 or S5 only (FIG. 22B; padj<0.05; DESeq2).

Analysis of the differentially expressed genes indicated that treatment with combination of S4 and S5 led to the induction of several different biological pathways. The biological pathways of genes significantly and at least 2 fold regulated in S4 [5 μg/mL]+S5 [6 μg/mL] treatment versus control in My-La and/or HuT-78 cell lines are shown in Table 12 hereinbelow. Involved pathways include, among others, PI3K-AKT pathway, pathways in cancer and cytokine/chemokine-receptor interaction pathways.

The steady state RNA levels, determined by qPCR, of some of the genes differentially expressed based on RNA sequencing is presented in Table 13A hereinbelow. Specifically, NFKBIZ-1 (geneID 64332) was significant upregulation in HuT-78 cells, RRM2-9 (geneID 50484) significant downregulation in HuT-78 cells, SATB1 (geneID 6304) significant upregulation in HuT-78 cells and PIK3R3-1 significant downregulation in HuT-78 cells by

S4+S5 treatment were validated by qPCR. The transcription factors ATF4 (gene ID 468) and the PSEUDOKINASE TRIBBLES HOMOLOGUE 3 (TRIB3, gene ID 57761) were significantly induced by the S4+S5 treatment in the qPCR experiments as well. However the changes in expression of AKT1 (geneID 207) and KCNN4 (geneID 3783), both suggested by the array to be repressed by S4+S5 treatment, could not be validate by qPCR.

Examination of steady state RNA levels of some of the differentially expressed genes in SPBL following S4 [5 μg/mL]+S5 [6 μg/mL] treatment versus the methanol control (Table 13B hereinbelow) revealed that SATB1 (geneID 6304) was upregulated in all examined SPBL. However, only slight changes in gene expression versus the control were recorded for NFKBIZ-1 (geneID 64332), whereas RRM2-9 (geneID 50484) expression was slightly repressed in 2 out of 3 examined patients. PIK3R3-1 (geneID 8503) expression was slightly increased in PBL upon S4 [5 μg/mL]+S5 [6 μg/mL] treatment.

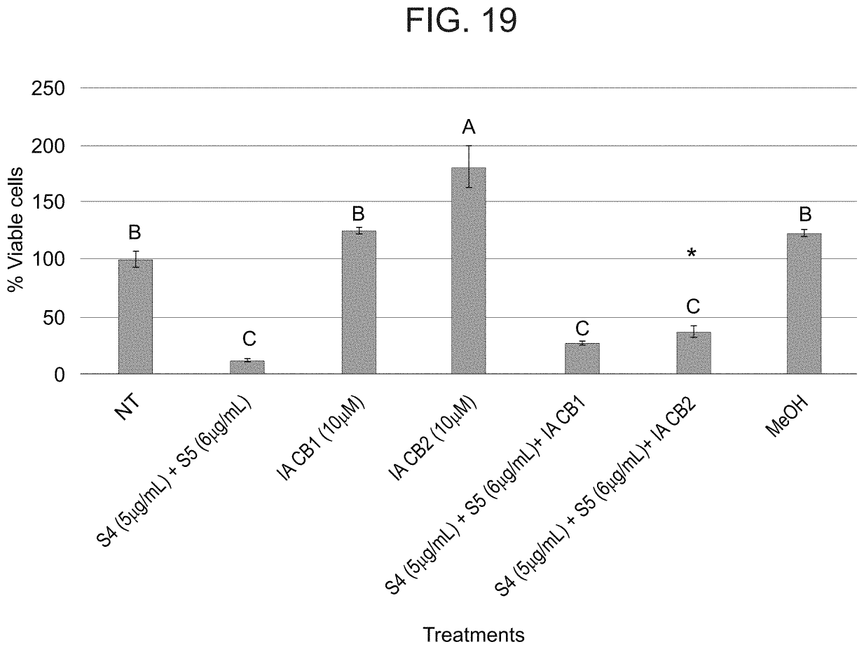

Moreover, adding a CB2 receptor inverse agonist to MyLa cancer cells 1 hour prior to treatment with S4 [5 μg/mL]+S5 [6 μg/mL] significantly reduced the cytotoxic activity of the fractions (FIG. 19), suggesting that S4 and S5 activity involves activation of CB2 receptors. However, adding CB1 receptor inverse agonist to this treatment did not significantly change S4 [5 μg/mL]+S5 [6 μg/mL] activity.

The chemical compositions of SCBD whole extract and the active fractions S4 and S5 were then analyzed by Gas chromatograph (GC) with mass selective detector (MSD) (GC/MS, FIGS. 7 and 8). Tables 2-4 hereinbelow detail the identified compounds and their ratios in each of these fractions. Table 5 hereinbelow details the ratios of the identified compounds in the S4 and S5 combinations which showed a synergistic effect as detailed hereinabove.

TABLE 2
Proportion of compounds identified in
SCBD whole extract based on GC/MS profile

| Compound | % |
|---|---|
| a-Pinene | 1.31 |
| b-Pinene | 0.12 |
| b-Caryophyllene | 0.44 |
| trans-a-Bergamontene | 0.16 |
| Humulene | 0.14 |
| a-Bisabolene | 0.23 |
| Selina-3,7-diene | 0.42 |
| Guaiol | 0.35 |
| a-Eudesmol | 0.38 |
| c-Eudesmol | 0.19 |
| Guaienol | 0.52 |
| a-Bisbolol | 0.31 |
| CBC | 0.16 |
| Unknown | 0.64 |
| CBD | 80.25 |
| Unknown | 1.47 |
| THC | 3.03 |
| CBG | 9.67 |
| alkane | 0.2 |

TABLE 3
Proportion of compounds
identified in S4 based on GC/MS profile

| % | compound |
|---|---|
| 98.3 | CBD |
| 0.3 | THC |
| 0.2 | CBG |
| 0.9 | a-bisabolol |
| 0.1 | CBDV |

TABLE 4
Proportion of compounds
identified in S5 based on GC/MS profile

| % | compound |
|---|---|
| 38.25 | CBD |
| 0.44 | CBC |
| 0.74 | THC |
| 58.85 | CBG |

TABLE 5
Combined S4 + S5 content for synergistic interactions,.

| | | | Molecular |
|---|---|---|---|
| Molar ratio | % | compound | weight (g/mol) |
| 1 | 58.8-87.6 | CBD | 314.46 |
| 0.2-0.6 | 11.7-39.2 | CBG | 316.48 |
| 0.003-0.006 | 0.15-0.49 | THC | 314.45 |
| 0.0015-0.003 | 0.09-0.29 | CBC | 314.47 |

TABLE 11
Cell Apoptosis of PBLs of healthy individuals
and Sezary patients following treatment with S4 and S5

| | | % Total | % late | % early |
|---|---|---|---|---|
| | Treatment | apoptosis | apoptosis | apoptosis |
| Healthy | MeOH | 13.7 | 2.7 | 11 |
| individual | S4 | 28.5 | 8.9 | 19.6 |
| NL3 | S5 | 33.8 | 6.3 | 27.5 |
| | S4 + S5 | 38.8 | 4.4 | 34.4 |
| Healthy | MeOH | 16.7 | 0 | 16.7 |
| individual | S4 | 20.2 | 5 | 15.2 |
| NL4 | S5 | 39.4 | 10.7 | 28.7 |
| | S4 + S5 | 65.1 | 27.2 | 37.9 |
| Sezary | MeOH | 12.3 | 3.3 | 9 |
| Patient | S4 | 47 | 14 | 33 |
| SZ13 | S5 | 40 | 19.3 | 20.7 |
| | S4 + S5 | 32.8 | 16.8 | 16 |
| Sezary | MeOH | 32.8 | 16.8 | 16 |
| Patient | S4 | 44.4 | 22.8 | 21.6 |
| SZ14 | S5 | 64.8 | 47.4 | 17.4 |
| | S4 + S5 | 54.6 | 32.6 | 22 |

TABLE 12
Biological pathways of genes significantly and at least 2 fold regulated in S4 [5
μg/mL] + S5 [6 μg/mL] treatment versus control in My-La and/or HuT-78 cell lines.

| | | Background | Input | | Input | |
|---|---|---|---|---|---|---|
| | | number | number | Corrected | number | Corrected |
| Pathway | ID | of genes | My-La | P-Value | HuT-78 | P-Value |
| Metabolic pathways | hsa01100 | 1243 | 257 | 6.08E−31 | 220 | 1.22E−31 |
| Parkinson's disease | hsa05012 | 142 | 50 | 6.82E−13 | 20 | 0.015419 |
| Non-alcoholic fatty liver | hsa04932 | 151 | 47 | 1.20E−10 | 28 | 8.42E−05 |
| disease (NAFLD) | | | | | | |
| Carbon metabolism | hsa01200 | 113 | 40 | 1.71E−10 | 23 | 0.000137 |
| Alzheimer's disease | hsa05010 | 168 | 49 | 2.91E−10 | 26 | 0.001711 |
| Oxidative phosphorylation | hsa00190 | 133 | 43 | 3.23E−10 | 17 | 0.050157 |
| RNA transport | hsa03013 | 172 | 49 | 5.65E−10 | 35 | 1.37E−06 |
| Epstein-Barr virus infection | hsa05169 | 204 | 54 | 6.04E−10 | 44 | 9.66E−09 |
| Huntington's disease | hsa05016 | 193 | 52 | 7.98E−10 | 31 | 0.000309 |
| Pathways in cancer | hsa05200 | 397 | 81 | 1.04E−09 | 47 | 0.002757 |
| Purine metabolism | hsa00230 | 176 | 49 | 1.08E−09 | 38 | 1.17E−07 |
| Ubiquitin mediated | hsa04120 | 137 | 42 | 2.01E−09 | 29 | 7.19E−06 |
| proteolysis | | | | | | |
| Pyrimidine metabolism | hsa00240 | 105 | 36 | 3.29E−09 | 29 | 7.25E−08 |
| Endocytosis | hsa04144 | 260 | 59 | 1.19E−08 | 48 | 1.19E−07 |
| Protein processing in | hsa04141 | 166 | 44 | 3.01E−08 | 31 | 2.77E−05 |
| endoplasmic reticulum | | | | | | |
| MAPK signaling pathway | hsa04010 | 255 | 55 | 1.71E−07 | 31 | 0.012631 |
| FoxO signaling pathway | hsa04068 | 134 | 37 | 2.00E−07 | 31 | 6.45E−07 |
| RNA degradation | hsa03018 | 77 | 27 | 2.99E−07 | 18 | 0.000223 |
| AMPK signaling pathway | hsa04152 | 125 | 35 | 3.48E−07 | 21 | 0.002375 |
| Viral carcinogenesis | hsa05203 | 205 | 47 | 3.67E−07 | 29 | 0.002807 |
| HTLV-I infection | hsa05166 | 259 | 54 | 5.62E−07 | 40 | 6.70E−05 |
| Thyroid hormone signaling | hsa04919 | 118 | 32 | 2.14E−06 | 19 | 0.005946 |
| pathway | | | | | | |
| Longevity regulating pathway | hsa04211 | 94 | 28 | 2.42E−06 | 20 | 0.00026 |
| Insulin signaling pathway | hsa04910 | 139 | 35 | 2.64E−06 | 28 | 2.38E−05 |
| Hepatitis B | hsa05161 | 146 | 36 | 2.75E−06 | 23 | 0.002832 |
| Prostate cancer | hsa05215 | 89 | 27 | 2.86E−06 | 16 | 0.005264 |
| mTOR signaling pathway | hsa04150 | 154 | 37 | 3.24E−06 | 27 | 0.000259 |
| Spliceosome | hsa03040 | 134 | 34 | 3.27E−06 | 37 | 7.33E−10 |
| @ @ Apoptosis | hsa04210 | 140 | 34 | 7.28E−06 | 23 | 0.001776 |
| HIF-1 signaling pathway | hsa04066 | 103 | 28 | 1.01E−05 | 21 | 0.000286 |
| Renal cell carcinoma | hsa05211 | 67 | 22 | 1.07E−05 | 16 | 0.000463 |
| Chronic myeloid leukemia | hsa05220 | 73 | 23 | 1.11E−05 | 12 | 0.029217 |
| Chemokine signaling pathway | hsa04062 | 187 | 40 | 1.29E−05 | 27 | 0.003187 |
| Small cell lung cancer | hsa05222 | 86 | 25 | 1.32E−05 | 13 | 0.036909 |
| Acute myeloid leukemia | hsa05221 | 57 | 20 | 1.37E−05 | 11 | 0.016139 |
| Endometrial cancer | hsa05213 | 52 | 19 | 1.51E−05 | 10 | 0.02301 |
| Hepatitis C | hsa05160 | 133 | 32 | 1.67E−05 | 19 | 0.016567 |
| Proteoglycans in cancer | hsa05205 | 205 | 42 | 1.82E−05 | 27 | 0.009136 |
| Choline metabolism in cancer | hsa05231 | 101 | 27 | 1.93E−05 | 15 | 0.027499 |
| cAMP signaling pathway | hsa04024 | 199 | 41 | 2.09E−05 | 18 | 0.273489 |
| Regulation of actin | hsa04810 | 215 | 43 | 2.33E−05 | 26 | 0.024702 |
| cytoskeleton | | | | | | |
| Measles | hsa05162 | 136 | 32 | 2.43E−05 | 28 | 1.68E−05 |
| B cell receptor signaling | hsa04662 | 73 | 22 | 3.13E−05 | 11 | 0.055789 |
| pathway | | | | | | |
| Colorectal cancer | hsa05210 | 62 | 20 | 3.60E−05 | 12 | 0.011312 |
| Adherens junction | hsa04520 | 74 | 22 | 3.68E−05 | 10 | 0.107928 |
| Ras signaling pathway | hsa04014 | 228 | 44 | 3.79E−05 | 31 | 0.003236 |
| Fc gamma R-mediated | hsa04666 | 93 | 25 | 3.85E−05 | 17 | 0.003398 |
| phagocytosis | | | | | | |
| Longevity regulating pathway- | hsa04213 | 64 | 20 | 5.16E−05 | 13 | 0.005815 |
| multiple species | | | | | | |
| Ribosome biogenesis in | hsa03008 | 89 | 24 | 5.47E−05 | 24 | 1.78E−06 |
| eukaryotes | | | | | | |
| Insulin resistance | hsa04931 | 109 | 27 | 5.75E−05 | 16 | 0.024129 |
| RNA polymerase | hsa03020 | 32 | 14 | 5.87E−05 | 14 | 6.45E−06 |
| Rap1 signaling pathway | hsa04015 | 211 | 41 | 6.43E−05 | 26 | 0.020923 |
| Pancreatic cancer | hsa05212 | 66 | 20 | 7.25E−05 | 10 | 0.067965 |
| T cell receptor signaling | hsa04660 | 105 | 26 | 8.17E−05 | 19 | 0.002011 |
| pathway | | | | | | |
| Central carbon metabolism in | hsa05230 | 67 | 20 | 8.57E−05 | 13 | 0.008001 |
| cancer | | | | | | |
| Neurotrophin signaling | hsa04722 | 120 | 28 | 9.70E−05 | 25 | 4.59E−05 |
| pathway | | | | | | |
| Herpes simplex infection | hsa05168 | 186 | 37 | 0.000105 | 35 | 6.20E−06 |
| Biosynthesis of amino acids | hsa01230 | 75 | 21 | 0.000115 | 17 | 0.00048 |
| Platinum drug resistance | hsa01524 | 75 | 21 | 0.000115 | 13 | 0.0165 |
| Phospholipase D signaling | hsa04072 | 144 | 31 | 0.000132 | 16 | 0.123806 |
| pathway | | | | | | |
| @ @ Cell cycle | hsa04110 | 124 | 28 | 0.000155 | 27 | 1.05E−05 |
| Aminoacyl-tRNA | hsa00970 | 66 | 19 | 0.000197 | 15 | 0.001101 |
| biosynthesis | | | | | | |
| PI3K-Akt signaling pathway | hsa04151 | 342 | 55 | 0.000248 | 38 | 0.017709 |
| Non-small cell lung cancer | hsa05223 | 56 | 17 | 0.000282 | 10 | 0.032836 |
| Progesterone-mediated oocyte | hsa04914 | 98 | 23 | 0.000442 | 16 | 0.011219 |
| maturation | | | | | | |

* Data was sorted based on corrected P-Value (calculated based on KOBAS www(dot)kobas(dot)cbi(dot)pku(dot)edu(dot)cn/index.php) of >0.005 in MyLa cell line. Annotation is based on KEGG Mapper (www(dot)genome(dot)jp/kegg/tool/map_pathway2(dot)html). Calculation of pvalue & correct pvalubased on www(dot)kobas(dot)cbi(dot)pku(dot)edu(dot)cn/index(dot)php (KOBAS).

TABLE 13A
Quantitative PCR determination the steady state
level of RNA in My-La and HuT-78 cell lines treated
with a combination of S4 and S5 of genes that were found
to be differentially expressed in RNA sequencing experiments.

| | | Steady state level | Significantly |
|---|---|---|---|
| Cell | | of RNA of S4 + S5 | changed in |
| line | Gene | treated versus control | comparison to control |
| My-La | NFKBIZ-1 | 0.84 ± 0.01 | yes |
| HuT-78 | NFKBIZ-1 | 3.93 ± 0.22 | yes |
| My-La | RRM2-9 | 1.07 ± 0.03 | no |
| HuT-78 | RRM2-9 | 0.66 ± 0.00 | yes |
| My-La | SATB1 | 1.02 ± 0.03 | no |
| HuT-78 | SATB1 | 3.95 ± 0.68 | yes |
| My-La | PIK3R3-1 | 1.41 ± 0.05 | yes |
| HuT-78 | PIK3R3-1 | 0.58 ± 0.03 | yes |
| My-La | AKT-1 | 1.43 ± 0.11 | yes |
| HuT-78 | AKT-1 | 1.63 ± 0.04 | yes |
| My-La | KCNN4-0 | 1.99 ± 0.14 | yes |
| HuT-78 | KCNN4-0 | 1.20 ± 0.04 | yes |
| My-La | ATF4-3 | 1.47 ± 0.02 | yes |
| HuT-78 | ATF4-3 | 3.00 ± 0.20 | yes |
| My-La | TRIB3-3 | 1.87 ± 0.07 | yes |
| HuT-78 | TRIB3-3 | 14.95 ± 1.09 | yes |

* Values of gene transcripts were determined by quantitative PCR as a ratio between target genes versus a reference gene (hypoxanthine phosphoribosyltransferase, HPRT, EC 2.4.2.8). Values for My-La or HuT-78 cells treated with S4 [5 μg/mL]+S5 [6 μg/mL] were calculated relative to the average expression of target genes in My-La or HuT-78 methanol treated control, respectively, using the 2^{ΔΔCt} method. ND− not determined.

TABLE 13B
The steady state RNA level in SPBL treated with a combination of S4 and S5
of genes that were found to be differentially expressed in RNA sequencing experiments.

| Patient | SATB1 | NFKBIZ-1 | RRM2-9 | PIK3R3-1 |
|---|---|---|---|---|
| designation/target | Gene ID | Gene ID | Gene ID | Gene ID |
| gene | 6304 | 64332 | 50484 | 8503 |
| Sz-11 | ND | ND | ND | ND |
| Sz-13 | 1.23 ± 0.10 | 0.96 ± 0.14 | 0.78 + 0.07 | 1.03 ± 0.09 |
| Sz-14 | 5.2 ± 0.38 | 0.90 ± 0.15 | 0.72 ± 0.02 | 1.19 ± 0.01 |
| Sz-15 | 1.2 ± 0.32 | 1.04 ± 0.09 | 1.07 ± 0.03 | 1.45 ± 0.26 |

*Values of gene transcripts were determined by quantitative PCR as a ratio between target genes versus a reference gene (hypoxanthine phosphoribosyltransferase, HPRT, EC 2.4.2.8). Values for SPBLs treated with S4 [5 μg/mL]+S5 [6 μg/mL] were calculated relative to the average expression of target genes in SPBL methanol treated control, using the 2^{ΔΔCt} method. ND—not determined.

### Example 2

### Combinations of Pure Cannabinoids in Specific Concentrations have Synergistic Activity in Reducing Cell Viability in Cutaneous T Cell Lymphoma Cell Lines

The cytotoxic activity of pure cannabinoids at the concentrations present in the combined active fractions S4 and S5 composition (see Table 5 hereinabove) was examined.

To this end, activity of each cannabinoid was examined separately, followed by examination of the cytotoxic effect of their combinations (FIGS. 23A-B). The results demonstrated that treatment with CBD only in concentration equal to that in the combined S4+S5 composition did not induce cell death of My-La cells (FIG. 2A). However, once CBD was combined with CBG, and especially the combination of CBD+CBG+THC or CBD+CBG+THC+CBC [at concentrations equivalent to their concentrations in the combined S4 (5 μg/mL) and S5 (6 μg/mL) composition], cytotoxicity was markedly increased; and was to treatment with S4+S5 (FIG. 23A).

When tested on HuT-78 cells, treatment with CBD only in concentration equal to that in the combined S4+S5 composition was potent and led to 50% cell death. However, similarly to the findings with My-La cells, a combination of CBD+CBG, CBD+CBG+CBC, CBD+CBG+THC or CBD+CBG+THC+CBC, had a cytotoxic activity similar to that of the S4+S5 treatment (FIG. 23B).

These results suggest that the synergistic cytotoxic effect of S4+S5 treatment results from the specific combination of the different cannabinoids.

### Example 3

### Extracts from C. Sativa Strain DQ Inflorescences are Active in Reducing Cell Viability in Cutaneous T Cell Lymphoma Cell Lines

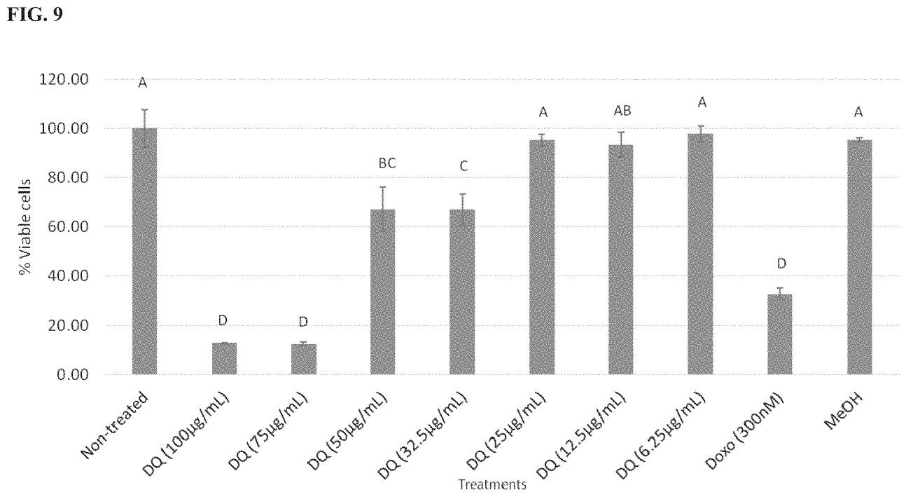

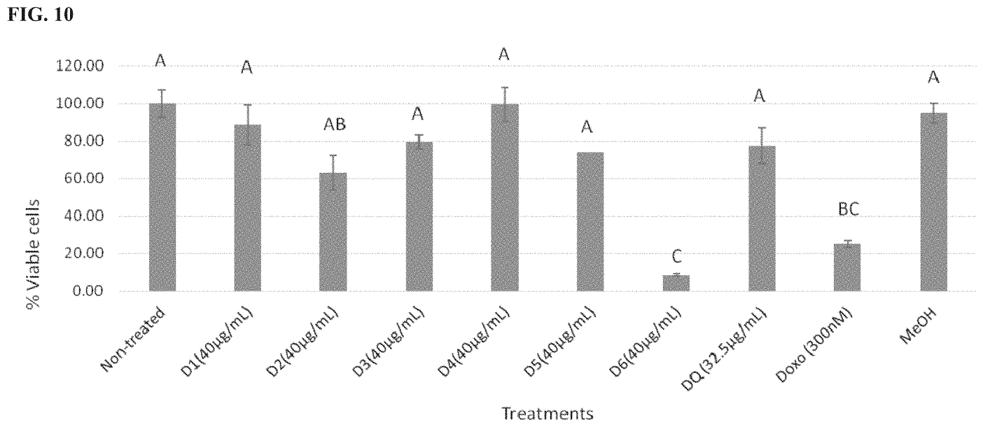

Cytotoxic activity was determined as the level of cell viability in MyLa cells for extracts of fresh inflorescences of C. sativa strain DQ following 48 hours of treatment. Treatment with the extracts reduced MyLa cancer cell viability in a dose dependent manner (FIG. 9).

Following, the extracts were fractionated by HPLC. Several fractions were collected and high concentrations and each was examined for cytotoxic activity on MyLa cancer cells.

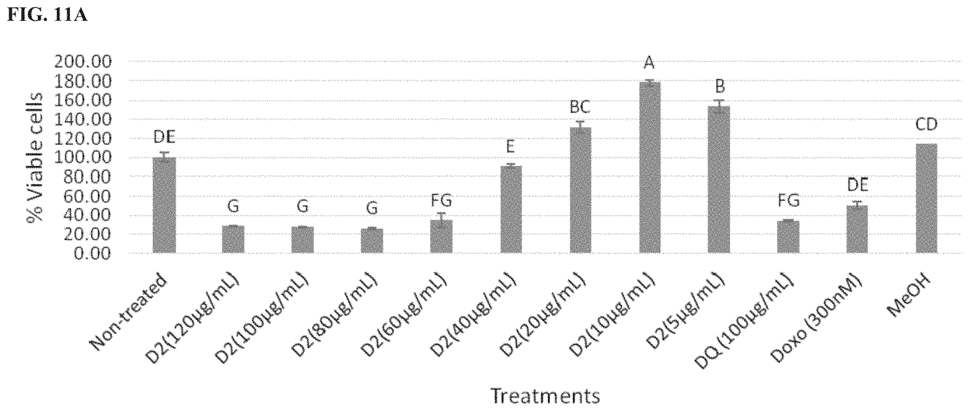

Fractions denoted herein as D2 and D6 were the most active fractions. In the case of D6 the activity was higher than the whole DQ extract (at a concentration of 32.5 μg/ml), and similar to the activity of 300 n g/ml of doxorubicin (FIG. 10).

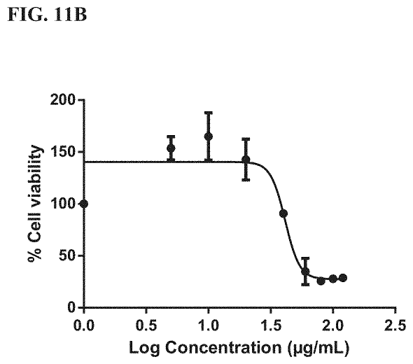

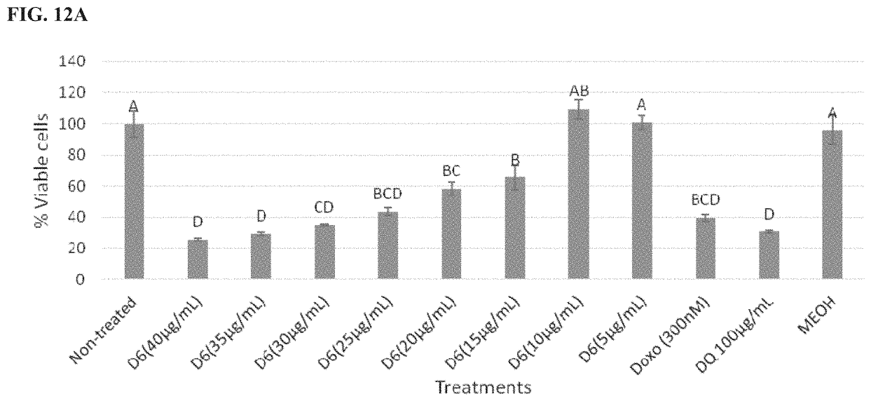

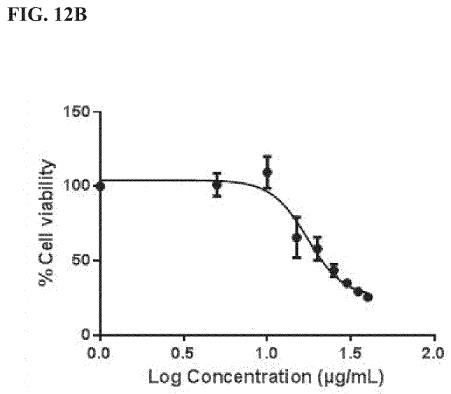

Moreover, treatment with each of D2 or D6 reduced MyLa cancer cell viability in a dose dependent manner, with an IC50 of 41.3 μg/ml and 17.4 μg/ml, respectively (FIGS. 11A-B and 12A-B).

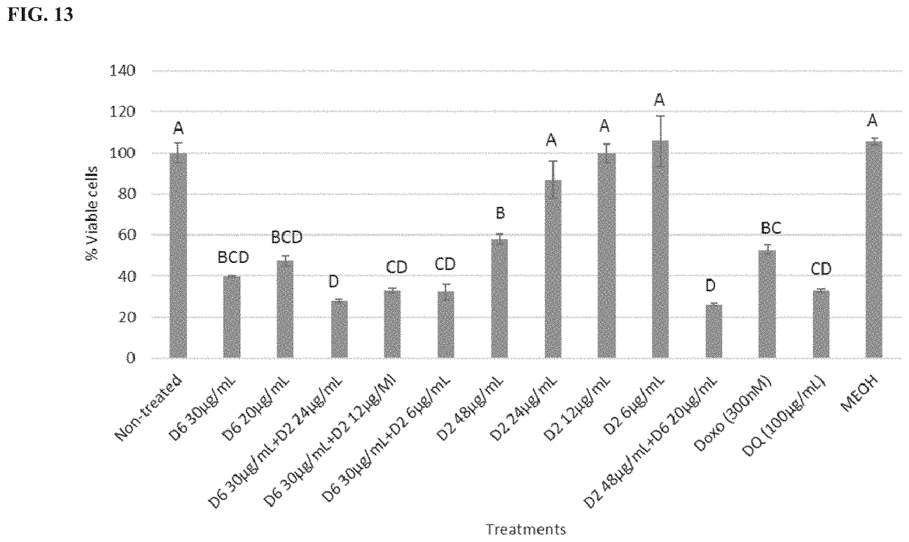

Interestingly, a combined treatment with D2 and D6 had a synergistic cytotoxic activity on MyLa cancer cells (FIG. 13). The combinations were also significantly more active than doxorubicin (300 nM). Next, the partial effect of the fractions was calculated according to the Bliss independence model for each combination experiment. As shown in Table 6 below the Bliss model calculations and indicated that combinations of D2 and D6 are synergistic.

TABLE 6
Synergy calculations of cytotoxic activity
(based on Bliss Model) of D2 and D6 combinations on MyLa cells

| | D6 30 μg/ml | | |
|---|---|---|---|
| | D2 24 μg/ml | D2 12 μg/ml | D2 6 μg/ml |
| Calculated value (μg/ml) | 34.54 | 39.65 | 46.55 |
| Experimental value (μg/ml) | 28.10 | 32.90 | 32.32 |
| | D2 48 μg/ml | | |
| | D6 20 μg/ml | D6 15 μg/ml | D6 10 μg/ml |
| Calculated value (μg/ml) | 27.54 | 33.43 | 43.19 |
| Experimental value (μg/ml) | 26.11 | 37.00 | 43.91 |

The chemical compositions of DQ whole extract and the active fractions D2 and D6 were then analyzed by GC/MS (FIGS. 14 and 15). Tables 7-9 hereinbelow detail the identified compounds and their ratios in each of these fractions. Table hereinbelow details the ratios of the identified compounds in the D2 and D6 combinations which showed a synergistic effect as detailed hereinabove.

TABLE 7
Proportion of compounds identified in
SCBD whole extract based on GC/MS profile

| % | Compound |
|---|---|
| 0.27 | a-Pinene |
| 0.08 | Camphene |
| 0.46 | b-Pinene |
| 0.75 | b-Myrcene |
| 3.94 | D-Limonene |
| 0.63 | Linalool |
| 0.22 | Fenchol |
| 0.01 | Trans-2-Pinanol |
| 0.05 | Borneol |
| 0.17 | a-Terpinol |
| 3.41 | b-Caryophyllene |
| 0.23 | Trans-a-Bergamotene |
| 0.79 | a-Elemene |
| 0.07 | Isoledene |
| 1.76 | Hunulene |
| 0.64 | a-Farnesene |
| 0.76 | Gernacene |
| 0.47 | Guaiol |
| 0.53 | c-Eudesmol |
| 0.73 | a-Eudesmol |
| 0.14 | Guaiene |
| 0.11 | Bisabolol |
| 1.37 | CBD |
| 78.88 | THC |
| 2.58 | CBG |
| 0.94 | CBN |

TABLE 8
Proportion of compounds identified
in D2 based on GC/MS profile

| % | Compound |
|---|---|
| 0.682644 | c-Eudesmol |
| 2.595529 | Longifolene |
| 2.334026 | Agarospirol |
| 2.757124 | Neoisolongifolene |
| 0.733196 | Ledol |
| 3.032741 | Germacrene D |
| 0.680642 | c-Maaliene |
| 1.176891 | bisabolene |
| 0.295467 | caryophyllene oxide |
| 0.447716 | Longifolenaldehyde |
| 3.095059 | CBC |
| 16.68778 | CBD |
| 6.447165 | CBG |
| 18.23 | Unknown |
| 40.76 | Non active components |

TABLE 9
Proportion of compounds identified
in D6 based on GC/MS profile

| % | Compound |
|---|---|
| 0.01 | β-caryophyllene |
| 50.6 | THC |
| 46.85 | THCA |
| 1.72 | CBN |
| 0.28 | Non-active components |

TABLE 10
Combined D2 + D6 content for synergistic interactions

| % | compound |
|---|---|
| 0.11-0.48 | c-Eudesmol |
| 0.43-1.83 | Longifolene |
| 0.39-1.65 | Agarospirol |
| 0.46-1.95 | Neoisolongifolene |
| 0.12-0.52 | Ledol |
| 0.51-2.14 | Germacrene D |
| 0.11-0.48 | c-Maaliene |
| 0.2-0.83 | bisabolene |
| 0.05-0.24 | caryophyllene oxide |
| 0.07-0.32 | Longifolenaldehyde |
| 0.52-2.18 | CBC |
| 2.78-11.78 | CBD |
| 1.07-4.55 | CBG |
| 0.00-0.01 | β-caryophyllene |
| 14.88-42.17 | THC |
| 13.78-39.04 | THCA |
| 0.5-1.42 | CBN |

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

### REFERENCES

### Other References are Cited Throughout the Application

[1] Merlin M D. Archaeological evidence for the tradition of psychoactive plant use in the old world. Economic Botany. 2003; 57: 295-323.
[2] Mechoulam R, Parker L A, Gallily R. Cannabidiol: an overview of some pharmacological aspects. The Journal of Clinical Pharmacology. 2002; 42: 11S-9S. doi
[3] Hanuš L O, Meyer S M, Muñoz E, Taglialatela-Scafati O, Appendino G. Phytocannabinoids: a unified critical inventory. Natural Product Reports. 2016; 33: 1357-92.
[4] Aizpurua-Olaizola O, Soydaner U, Öztürk E, Schibano D, Simsir Y, Navarro P, Etxebarria N, Usobiaga A. Evolution of the cannabinoid and terpene content during the growth of Cannabis sativa plants from different chemotypes. Journal of Natural Products. 2016; 79: 324-31.
[5] Flores-Sanchez I J, Verpoorte R. Secondary metabolism in cannabis. Phytochemistry Reviews. 2008; 7: 615-39.
[6] Pertwee R G, Howlett A, Abood M E, Alexander S, Di Marzo V, Elphick M, Greasley P, Hansen H S, Kunos G, Mackie K. International Union of Basic and Clinical Pharmacology. LXXIX. Cannabinoid receptors and their ligands: beyond CB1 and CB2. Pharmacological Reviews. 2010; 62: 588-631.
[7] Maccarrone M, Bab I, Bíró T, Cabral G A, Dey S K, Di Marzo V, Konje J C, Kunos G, Mechoulam R, Pacher P. Endocannabinoid signaling at the periphery: 50 years after THC. Trends in Pharmacological Sciences. 2015; 36: 277-96.
[8] Maccarrone M, Di Rienzo M, Battista N, Gasperi V, Guerrieri P, Rossi A, Finazzi-Agró A. The Endocannabinoid System in Human Keratinocytes: Evidence that anandamide inhibits epidermal differentiation through CB1 receptor-dependent inhibition of protein kinase C, activating protein-1, and transglutaminase. Journal of Biological Chemistry. 2003; 278: 33896-903.
[9] Galiègue S, Mary S, Marchand J, Dussossoy D, Carrière D, Carayon P, Bouaboula M, Shire D, L E Fur G, Casellas P. Expression of central and peripheral cannabinoid receptors in human immune tissues and leukocyte subpopulations. European Journal of Biochemistry. 1995; 232: 54-61.
[10] Börner C, Bedini A, Hölt V, Kraus J. Analysis of promoter regions regulating basal and interleukin-4-inducible expression of the human CB1 receptor gene in T lymphocytes. Molecular Pharmacology. 2008; 73: 1013-9.
[11] Willemze R, Cerroni L, Kempf W, Berti E, Facchetti F, Swerdlow S H, Jaffe E S. The 2018 update of the WHO-EORTC classification for primary cutaneouslymphomas. Blood. 2019; 133:1703-14.
[12] Willemze R, Cerroni L, Kempf W, Berti E, Facchetti F, Swerdlow S H, Jaffe E S. The 2018 update of the WHO-EORTC classification for primary cutaneous lymphomas. Blood. 2019; 133: 1703-14.
[13] Prince H M, Querfeld C. Integrating novel systemic therapies for the treatment of mycosis fungoides and Sézary syndrome. Best Practice & Research Clinical Haematology. 2018; 31: 322-35.
[14] Jawed S I, Myskowski P L, Horwitz S, Moskowitz A, Querfeld C. Primary cutaneous T-cell lymphoma (mycosis fungoides and Sézary syndrome): part I. Diagnosis: clinical and histopathologic features and new molecular and biologic markers. Journal of the American Academy of Dermatology. 2014; 70: 205. e1-. e16.
[15] Javid F A, Phillips R M, Afshinjavid S, Verde R, Ligresti A. Cannabinoid pharmacology in cancer research: A new hope for cancer patients? European Journal of Pharmacology. 2016; 775: 1-14.
[16] Scott K A, Dalgleish A G, Liu W M. Anticancer effects of phytocannabinoids used with chemotherapy in leukaemia cells can be improved by altering the sequence of their administration. International Journal of Oncology. 2017; 51: 369-77.
[17] Scott K A, Shah S, Dalgleish A G, Liu W M. Enhancing the activity of cannabidiol and other cannabinoids in vitro through modifications to drug combinations and treatment schedules. Anticancer Research. 2013; 33: 4373-80.
[18] Russo E B. The Case for the Entourage Effect and Conventional Breeding of Clinical Cannabis: No “Strain,” No Gain. Frontiers in Plant Science. 2018; 9: 1969.
[19] Nallathambi R, Mazuz M, Namdar D, Shik M, Namintzer D, Vinayaka A C, Ion A, Faigenboim A, Nasser A, Laish I. Identification of synergistic interaction between cannabis-derived compounds for cytotoxic activity in colorectal cancer cell lines and colon polyps that induces apoptosis-related cell death and distinct gene expression. Cannabis and Cannabinoid Research. 2018; 3: 120-35.
[20] Bernengo M G, Novelli M, Quaglino P, Lisa F, De Matteis A, Savoia P, Cappello N, Fierro M T. The relevance of the CD4+ CD26—subset in the identification of circulating Sézary cells. British Journal of Dermatology. 2001; 144: 125-35.
[21] Velasco G, Sánchez C, Guzmán M. Anticancer mechanisms of cannabinoids. Current Oncology. 2016; 23: S23.
[22] (Carracedo A, Lorente M, Egia A, Blázquez C, García S, Giroux V, Malicet C, Villuendas R, Gironella M, González-Feria L, Piris M Á. The stress-regulated protein p8 mediates cannabinoid-induced apoptosis of tumor cells. Cancer cell. 2006 Apr. 1; 9(4):301-12.)
[23] Gustafsson K, Wang X, Severa D, Eriksson M, Kimby E, Merup M, Christensson B, Flygare J, Sander B. Expression of cannabinoid receptors type 1 and type 2 in non□Hodgkin lymphoma: Growth inhibition by receptor activation. International Journal of Cancer. 2008; 123: 1025-33.
[24] Islam T, Asplund A, Lindvall J, Nygren L, Liden J, Kimby E, Christensson B, Smith C, Sander B. High level of cannabinoid receptor 1, absence of regulator of G protein signalling 13 and differential expression of Cyclin D1 in mantle cell lymphoma. Leukemia. 2003; 17: 1880.
[25] Laprairie R, Bagher A, Kelly M, Denovan□ Wright E. Cannabidiol is a negative allosteric modulator of the cannabinoid CB1 receptor. British Journal of Pharmacology. 2015; 172: 4790-805.
[26] Guo X, Wang X-F. Signaling cross-talk between TGF-β/BMP and other pathways. Cell Research. 2009; 19: 71.
[27] Izban K F, Ergin M, Qin J-Z, Martinez R L, Pooley J R R J, Saeed S, Alkan S. Constitutive expression of NF-κB is a characteristic feature of mycosis fungoides: implications for apoptosis resistance and pathogenesis. Human Pathology. 2000; 31: 1482-90.
[28] Duxbury M S, Whang E E. RRM2 induces NF-κB-dependent MMP-9 activation and enhances cellular invasiveness. Biochemical and Biophysical Research Communications. 2007; 354: 190-6.
[29] Davis M E, Zuckerman J E, Choi C H J, Seligson D, Tolcher A, Alabi C A, Yen Y, Heidel J D, Ribas A. Evidence of RNAi in humans from systemically administered siRNA via targeted nanoparticles. Nature. 2010; 464: 1067.
[30] Alvarez J D, Yasui D H, Niida H, Joh T, Loh D Y, Kohwi-Shigematsu T. The MAR-binding protein SATB1 orchestrates temporal and spatial expression of multiple genes during T-cell development. Genes & Development. 2000; 14: 521-35.
[31] Kakugawa K, Kojo S, Tanaka H, Seo W, Endo T A, Kitagawa Y, Muroi S, Tenno M, Yasmin N, Kohwi Y. Essential roles of SATB1 in specifying T lymphocyte subsets. Cell Reports. 2017; 19: 1176-88.
[32] Jankowska-Konsur A, Kobierzycki C, Reich A, Grzegrzolka J, Bieniek A, Dziegiel P. Expression of SATB1, MTI/II and Ki-67 in mycosis fungoides. Anticancer Research. 2016; 36: 189-97.
[33] Fredholm S, Willerslev-Olsen A, Met Ö, Kubat L, Gluud M, Mathiasen S L, Friese C, Blümel E, Petersen D L, Hu T. SATB1 in malignant T cells. Journal of Investigative Dermatology. 2018; 138: 1805-15.
[34] Bacon C M, Petricoin E F, Ortaldo J R, Rees R C, Larner A C, Johnston J A, O'Shea J J. Interleukin 12 induces tyrosine phosphorylation and activation of STAT4 in human lymphocytes. Proceedings of the National Academy of Sciences. 1995; 92: 7307-11.
[35] Kaplan M H. STAT4. Immunologic Research. 2005; 31: 231-41.
[36] Chakraborty A R, Robey R W, Luchenko V L, Zhan Z, Piekarz R L, Gillet J-P, Kossenkov A V, Wilkerson J, Showe L C, Gottesman M M. MAPK pathway activation leads to Bim loss and histone deacetylase inhibitor resistance: rationale to combine romidepsin with an MEK inhibitor. Blood. 2013; 121: 4115-25.
[37] Blachly J S, Baiocchi R A. Targeting PI3□kinase (PI3K), AKT and mTOR axis in lymphoma. British Journal of Haematology. 2014; 167: 19-32.
[38] Yang Q, Modi P, Newcomb T, Quéva C, Gandhi V. Idelalisib: first-in-class PI3K delta inhibitor for the treatment of chronic lymphocytic leukemia, small lymphocytic leukemia, and follicular lymphoma. Clinical Cancer Research. 2015; 21: 1537-42.
[39] Stivala L A, Cazzalini O, Prosperi E. The cyclin-dependent kinase inhibitor p21CDKN1A as a target of anti-cancer drugs. Current Cancer Drug Targets. 2012; 12: 85-96.
[40] Kaltoft K, Bisballe S, Dyrberg T, Boel E, Rasmussen P B, Thestrup-Pedersen K. Establishment of two continuous T-cell strains from a single plaque of a patient with mycosis fungoides. In Vitro Cellular & Developmental Biology-Animal. 1992; 28: 161-7.
[41] Gazdar A, Carney D, Bunn P, Russell E, Jaffe E, Schechter G, Guccion J. Mitogen requirements for the in vitro propagation of cutaneous T-cell. Blood. 1980; 55: 409.
[42] Delaney W E, Yang H, Miller M D, Gibbs C S, Xiong S. Combinations of adefovir with nucleoside analogs produce additive antiviral effects against hepatitis B virus in vitro. Antimicrobial Agents and Chemotherapy. 2004; 48: 3702-10.
[43] Olsen E, Vonderheid E, Pimpinelli N, Willemze R, Kim Y, Knobler R, Zackheim H, Duvic M, Estrach T, Lamberg S. Revisions to the staging and classification of mycosis fungoides and Sezary syndrome: a proposal of the International Society for Cutaneous Lymphomas (ISCL) and the cutaneous lymphoma task force of the European Organization of Research and Treatment of Cancer (EORTC). Blood. 2007; 110: 1713-22.

1-8. (canceled)

9. A composition comprising 50-90% cannabidiol (CBD), 10-40% cannabigerol (CBG) and 0.15-0.5% tetrahydrocannabinol (THC).

10. The composition of claim 9, wherein said composition further comprises 0.09-0.3% cannabichromene (CBC).

11. The composition of claim 10, wherein a total concentration of said CBD, said CBG said THC and/or said CBC in said composition is as listed in Table 5.

12. A composition comprising cannabidiol (CBD), cannabigerol (CBG) and tetrahydrocannabinol (THC) in molar ratios as listed in Table 5.

13. The composition of claim 12, wherein said compositing further comprises cannabichromene (CBC) in a molar ratio as listed in Table 5.

14. A composition comprising at least two cannabinoids selected from the group consisting of cannabidiol (CBD), cannabichromene (CBC), cannabigerol (CBG), tetrahydrocannabinol (THC), tetrahydrocannabinolic acid (THCA) and cannabinol (CBN), wherein a concentration of said CBD, said CBC, said CBG, said THC, said THCA and/or said CBN in said composition is 2.5-12% CBD, 0.5-2.2% CBC, 1-5% CBG, 14-43% THC, 13-40% THCA and/or 0.5-1.5% CBN.

15. The composition of claim 14, wherein a concentration of said CBD, said CBC, said CBG, said THC, said THCA and/or said CBN in said composition is as listed in Table 10.

16. The composition of claim 9, wherein said composition comprises a liquid chromatography fraction of cannabis extract.

17. The composition of claim 9, wherein said composition is a synthetic composition.

18. (canceled)

19. The composition of claim 9, wherein said composition has a cytotoxic activity on cutaneous T cell lymphoma (CTCL) cells.

20. The composition of claim 9, wherein said composition has a combined synergistic cytotoxic activity on cutaneous T cell lymphoma cells (CTCL) as compared to each of said cannabinoids when administered as a single agent.

21. A method of treating an inflammatory disease in a subject in need thereof, the method comprising administering to the subject a therapeutically effective amount of the composition of claim 9, thereby treating the disease in the subject.

22. The method of claim 21, wherein said inflammatory disease is cancer.

23. The of claim 22, wherein said, wherein said cancer is lymphoma.

24. The method of claim 23, wherein said lymphoma is cutaneous T cell lymphoma (CTCL).

25-44. (canceled)

45. The composition of claim 14, wherein said composition comprises a liquid chromatography fraction of cannabis extract.

46. The composition of claim 14, wherein said composition is a synthetic composition.

47. The composition of claim 14, wherein said composition has a cytotoxic activity on cutaneous T cell lymphoma (CTCL) cells.

48. The composition of claim 14, wherein said composition has a combined synergistic cytotoxic activity on cutaneous T cell lymphoma cells (CTCL) as compared to each of said cannabinoids when administered as a single agent.

49. A method of treating an inflammatory disease in a subject in need thereof, the method comprising administering to the subject a therapeutically effective amount of the composition of claim 14, thereby treating the disease in the subject.

50. The method of claim 49, wherein said inflammatory disease is cancer.

51. The method of claim 50, wherein said, wherein said cancer is lymphoma.

52. The method of claim 51, wherein said lymphoma is cutaneous T cell lymphoma (CTCL).
